(19) United States
(12) Patent Application Publication
(10) Pub. No.: US 2016/0089377 A1
(43) Pub. Date: Mar. 31, 2016

(54) THERAPEUTIC TARGETING OF THE MTOR PATHWAY IN NEURODEVELOPMENTAL AND NEUROPSYCHIATRIC DISEASE

(71) Applicant: THE TRUSTEES OF COLUMBIA UNIVERISTY IN THE CITY OF NEW YORK, New York, NY (US)

(72) Inventors: Maria Karayiorgou, Riverdale, NY (US); Joseph A. Gogos, Riverdale, NY (US); Sander Markx, Brooklyn, NY (US)

(73) Assignee: THE TRUSTEES OF COLUMBIA UNIVERISTY IN THE CITY OF NEW YORK, New York, NY (US)

(21) Appl. No.: 14/850,471

(22) Filed: Sep. 10, 2015

Related U.S. Application Data
(63) Continuation of application No. PCT/US2014/003069, filed on Sep. 17, 2014.
(60) Provisional application No. 61/794,606, filed on Mar. 15, 2013.
(60) Provisional application No. 61/852,150, filed on Mar. 15, 2013.
(60) Provisional application No. 61/803,977, filed on Mar. 21, 2013.
(60) Provisional application No. 61/918,307, filed on Dec. 19, 2013.

(51) Int. Cl. A61K 31/5386; A61K 31/4375; A61K 31/5377

(52) U.S. Cl. CPC A61K 31/5386; A61K 31/5377; A61K 31/4375

(57) ABSTRACT

The present invention relates to methods of treating various neurodevelopmental and neuropsychiatric diseases which employ inhibition of the mTOR pathway, particularly using mTOR kinase inhibitors. It is based, at least in part, on extensive phenotypic characterization of a knock-out mouse model of Caspr2, the murine ortholog of CNTNAP2, which indicate that the mechanism via which CNTNAP2 deficits lead to neuropsychiatric disorders is overactivation of the mTOR pathway. Accordingly, the present invention provides for methods of treating subjects suffering from neurodevelopmental and/or neuropsychiatric disorders comprising administering, to the subject, an agent that inhibits the mTOR pathway. In particular non-limiting embodiments, the inhibitor of the mTOR pathway is a mTOR kinase inhibitor such as, but not limited to, WYE125132 and analogous compounds. In a non-limiting subset of embodiments, subjects may be tested to determine whether they have a copy number variation or mutation in CNTNAP2 and where such a copy number variation or mutation is present treatment with a mTOR pathway inhibitor may be initiated.

### PRIORITY CLAIM

This application is a continuation of International Patent Application No. PCT/US2014/030693 filed, Mar. 17, 2014, which claims priority to U.S. Provisional Application Ser. No. 61/794,606, filed Mar. 15, 2013; U.S. Provisional Application Ser. No. 61/852,150, filed Mar. 15, 2013; U.S. Provisional Application Ser. No. 61/803,977, filed Mar. 21, 2013; and U.S. Provisional Application Ser. No. 61/918,307, filed Dec. 19, 2013; the contents of each of which are incorporated by reference in their entireties herein, and priority to each of which is claimed.

### GRANT INFORMATION

Not applicable.

### 1. INTRODUCTION

The present invention relates to methods of treating various neurodevelopmental and neuropsychiatric diseases which employ inhibition of the mTOR pathway, particularly using mTOR kinase inhibitors.

### 2. BACKGROUND OF THE INVENTION

The CNTNAP2 encodes Contactin-associated protein-like 2 (CNTNAP2), a member of the Neurexin family of proteins. CNTNAP2 functions as a cell adhesion protein in the vertebrate nervous system, and mediates interactions between neurons and glia cells during nervous system development. In mouse models, the loss of CNTNAP2—known in the mouse as Caspr2—results in the abnormal migration of neurons, reduction in the number of interneurons, and abnormal neuronal network activity (Penagarikano et al., 2011). Recent studies have shown that CNTNAP2 is critical for proper potassium ion channel clustering to the juxtaparanode region of myelinated axons, and for formation of functionally distinct domains in neurons important for saltatory conduction of nerve impulses (Poliak et al., 2001, 2003).

Several recent studies have described intragenic copy number variations (CNVs) or mutations in CNTNAP2 in a number of patients with schizophrenia (SCZ; 1,2) and autism spectrum disorder (ASD; 3,4). Moreover, Strauss et al., described patients from the Old-Order Amish population who are homozygous for a frameshift mutation (the 3709delG mutation) within the CNTNAP2 gene, who presented with evidence of cortical dysplasia and abnormalities in neuronal migration (Strauss et al., 2006). These patients are typically born normal, but at age 1.5-2 years old undergo significant cognitive, language, and motor decline with a severe seizure disorder and ultimately develop ASD. Genetic variants of CNTNAP2 have also been identified in patients with a wide range of other neuropsychiatric disorders, including bipolar disorder (Wang et al., 2010), attention-deficit hyperactivity disorder (ADHD; Elia et al., 2010), Gilles de la Tourette, and obsessive-compulsive disorder (Verkerk et al., 2003). Together, these human genetics studies indicate that CNTNAP2 is a strong candidate gene for neuropsychiatric diseases, primarily ASD, SCZ and seizure disorder, and provides an excellent opportunity to model core aspects of neuropsychiatric phenotypes in mice.

### 3. SUMMARY OF THE INVENTION

The present invention relates to methods of treating various neurodevelopmental and neuropsychiatric diseases which employ inhibition of the mTOR pathway, particularly using mTOR kinase inhibitors. It is based, at least in part, on extensive phenotypic characterization of a knock-out mouse model of Caspr2, the murine ortholog of CNTNAP2, which indicates that the mechanism via which CNTNAP2 deficits lead to neuropsychiatric disorders is overactivation of the mTOR pathway.

Accordingly, the present invention provides for methods of treating subjects suffering from neurodevelopmental and/or neuropsychiatric disorders comprising administering, to the subject, an agent that inhibits the mTOR pathway. In particular non-limiting embodiments, the inhibitor of the mTOR pathway is a mTOR kinase inhibitor such as, but not limited to, a ATK-competitive inhibitor such as WYE125132, Torin 2, AZD2014, and analogous compounds. In a non-limiting subset of embodiments, subjects may be tested to determine whether they have a copy number variation or mutation in CNTNAP2 and where such a copy number variation or mutation is present treatment with a mTOR pathway inhibitor may be initiated.

In certain embodiments, the inhibitor of the mTOR pathway is administered to a subject in an amount effective to decrease the level of S6 phosphorylation.

In certain embodiments, the inhibitor of the mTOR pathway is administered to a subject in an amount effective to increase expression of a glutamate receptor subunit, for example, a GluR2 receptor subunit, an NR2A receptor subunit, or combinations thereof.

In certain embodiments, the inhibitor of the mTOR pathway is administered to a subject in an amount effective to decrease expression of a glutamate receptor subunit, for example, a GluR1 receptor subunit, an NR1 receptor subunit, an NR2B receptor subunit, an mGLUR1 receptor subunit, an mGLUR5 receptor subunit, or combinations thereof.

In certain embodiments, the inhibitor of the mTOR pathway is administered to a subject in an amount effective to increase social interaction and/or cognition.

In certain embodiments, the inhibitor of the mTOR pathway is administered to a subject in an amount effective to decrease the subject's susceptibility to seizures. In certain embodiments, the inhibitor of the mTOR pathway is administered to a subject in an amount effective to increase the subject's threshold for seizures, for example, increasing the subject's threshold to a seizure stimulant such as, for example, pilocarpine.

In certain embodiments, the inhibitor of the mTOR pathway is administered to a subject in an amount effective to reduce a symptom of a psychiatric disorder.

In certain embodiments, a subject having a neurodevelopmental or psychiatric disorder is identified as likely to benefit from treatment with an inhibitor of the mTOR pathway, for example a mTOR kinase inhibitor, by determining that the subject exhibits one or more of the following, for example, as demonstrated in a cell sample from the subject: increased phosphorylation of S6, decreased GluR2 receptor subunit, decreased NR2A receptor subunit, increased GluR1 receptor subunit, increased NR1 receptor subunit, increased NR2B receptor subunit, increased mGLUR1 receptor subunit, and/or increased mGLUR5 receptor subunit, relative to a normal control subject.

### 4. BRIEF DESCRIPTION OF THE DRAWINGS

FIG. 1. Prepulse inhibition (PPI). Mice heterozygous for the Caspr2 mutant allele showed a decrease in PPI compared to mice homozygous for the mutant allele as well as wild-type littermates.

FIG. 2A-C. Abnormalities in social interaction. (A) Caspr2^{-/-} HOM mice spent less time socially investigating than Caspr2^{+/-}/-HET mice which spent less time socially investigating than WT littermates. When dividing the 10-minute interval of time spent socially investigating into individual minutes, a statistically significant reduction in the HOMs compared to WTs is evident in the early 1-min intervals of the experiment. (B). When examining time spent sniffing as a social measure, we found that HOMs have significantly shorter bouts of sniffing compared to WT littermates (C).

FIG. 3A-E. Pyramidal cells in Caspr2 mutant mice (A-C) and human subjects with homozygous CNTNAP2 frameshift mutations. YFP fluorescence of pyramidalcells in layer 5 of FC of 2-month-old (A) WT, (B) Caspr2^{-/-} HOM and (C) Caspr2^{+/-} HET mice at 200×, demonstrating increased pyramidal soma size (white arrows) in both HOM and HET mice compared to WT littermates. Cresyl violet staining of TC tissue in a 17-year-old normal control (D) and 3 patients with homozygous CNTNAP2 mutations (E; at 60× demonstrating increased soma size with enlarged nucleus in all 3 patients (white arrows).

FIG. 4. Quantification of pyramidal cell size in Caspr2 mutant mice. 200× images were taken using an epifluorescent microscope of somatosensory and auditory cortex. Cell somata were traced on Photoshop to quantify pixels. Pixels were converted to square microns. Left panel demonstrates a statistically significant difference in cell size between WTs and HETs (P<0.0001) and WTs and HOMs (P<0.0001). The right panel shows individual pyramidal cell measurements ordered from smallest to largest cell size, with each group plotted as a line graph. The smallest pyramidal cells are the same size in all three 3 genotypes, but the largest pyramidal cells are much larger in HOMs (HOMs>HETs>WTs).

FIG. 5. Phosphorylation of the ribosomal S6 in the hippocampus of the Caspr2 mutant mice. Hippocampal homogenates from adult mice were probed with an antibody directed against the S6 S235/236 phosphorylation epitope. A ˜20% increase in phosphorylation was observed in HOM knock-out mice. Seven mice per genotype were analyzed.

FIG. 6. Pyramidal cells in the TC of human subjects with homozygous CNTNAP2 mutation. Co-staining for cresyl violet (blue) and phosphorylated S6 (pS6; brown). Right panel demonstrates an age-matched normal control with minimal staining for pS6 whereas the left panel demonstrates a dramatic pS6 staining in significantly enlarged pyramidal cells in the subjects with the homozygous CNTNAP2 mutation.

FIG. 7. Representative traces of spontaneous postsynaptic currents (PSCs) recorded from pyramidal neurons in FC layer 5. Plotted data of recordings from FC layer 5 pyramidal neurons, across all three genotypes: WT (blue), Caspr2^{-/-} HOM (red), and Caspr2^{+/-}

HET (gray) mice. The top graph shows the amplitude of spontaneous EPSCs for the three genotype groups, whereas the bottom graph shows the amplitude of spontaneous IPSCs. Each dot represents data from a single neuron, whereas the crossbar represents the average value (n=6 for WT and HET, n=7 for HOM; P<0.05 for the EPSCs between WT and HOM, unpaired t-test).

FIG. 8A-B. Transcranial in vivo two-photon imaging of Caspr2 mutant mice compared to WT littermates. Panel (A) demonstrates a reduction in spine formation in the Caspr2 mice whereas panel (B) shows a significant increase in spine elimination.

FIG. 9. Electron microscopy examination of synaptic structure in Caspr2^{-/-} compared to WT littermates. The left panel shows both perforated (lower red arrow) and non-perforated (upper red arrow) post-synaptic densities (PSDs) in layer 5 of the FC of a Caspr2^{-/-} HOM mouse. The right panel shows a bar graph, which indicated that the length of the perforated PSDs in Caspr2^{-/-} mice in layer 5 of the FC is reduced by ˜25%.

FIG. 10A-B. FDG Micro-PET-MRI imaging of baseline glucose metabolism in Caspr2 mutant mice. SPM results reveal areas of significant activation in mutants compared to the WT littermates group; areas of activation are shown in red (see arrows). Panel (A) demonstrates clearly the cortical and subcortical areas of FDG hypermetabolism in the Caspr2^{+/-} HET mice compared to WT littermates. Panel (B) shows a similar pattern of cortical and subcortical FDG hypermetabolism in the Caspr2^{-/-} HOM mice compared to WT littermates.

FIG. 11A-E. High resolution MM of Caspr2 mutants. A-C: The figure (A) shows the location of the segmented occipital lobe, while the bar graphs (B and C) show the difference in size of the occipital lobe in absolute terms (in mm3; B) and in relevant terms as a percentage of total brain volume (C). The relative volume differences held up to the multiple comparisons, with FDR values of 0.08 for the HET and 0.01 for the HOM. D and E show the areas of decrease within the occipital lobe. FDR is between 10% and <15%.

FIG. 12 depicts a summary of the data for vehicle- and WYE125132-treated animals and soma size.

FIG. 13. Spine elimination and spine formation in wild-type mice and mice homozygous or heterozygous for Caspr2, treated with WYE123132 or vehicle.

FIG. 14A-F. Elecrophysiologic recordings from iCaspr2 imutants and wild-type litermates treated with vehicle or drug. (A,C,E) Long-term potentiation in wild-type mice, knockout mice treated with vehicle, and knockout mice treated with drug, respectively. (B, D, F0 shows E:I correlations.

FIG. 15A-E. Behavioral and Neuroimaging Phenotypes of Cntnap2 Mutant Mice. (A) Cntnap2 mutant mice show social inhibition in the first minute (upper and middle panel) as well as a decrease social interaction over the whole 10-minute interval. (B) Cntnap2^{-/-} mice were found to have a lowered seizure threshold upon pilocarpine administration as indicated by a higher seizure stage reached by Cntnap2^{-/-} mice (right panel) as well as more time spent in stage 4 seizures by Cntnap2^{-/-} mice (left panel). Bars represent, left to right, wild type, Cntnap^{+/-} and Cntnap2^{-/-}. (C) MRI analysis demonstrated grossly normal mesoscopic neuroanatomy in Cntnap2 mutant mice. Regional differences found in the Cntnap2^{-/-} mouse brain were specific to the occipital cortex. Bar graphs representing these differences are shown for both absolute volume (in mm³; bar graph to the left) and relative volume (% total brain volume; bar graph to the right). Error bars represent 95% confidence intervals and significance is indicated as a measure of false discovery rate (FDR) with *representing an FDR of less than 10% and ** representing an FDR of less than 5%. Again, bars represent, left to right, wild type, Cntnap^{+/-} and Cntnap2^{-/-}. (D) Voxel-wise differences. No voxel-wise differences were found when the entire brain was examined. However, when the occipital cortex was analyzed independently to minimize the effect of the multiple comparisons, bilateral differences were found with FDRs ranging from 10 to 15%. The bar graph represents the relative volume difference found in the indicated voxel. Error bars represent 95% confidence intervals. Bars represent, left to right, wild type, Cntnap^{+/-} and Cntnap2^{-/-}. (E) Statistical Parametric Mapping (SPM) of FDG-microPET/MRI scan data from Cntnap2 mutants. Coronal images demonstrate hypermetabolism in cortical and subcortical structures in both Cntnap2^{+/-} (upper panel) and Cntnap2^{-/-} (lower panel) mice. In Cntnap2^{+/-} mice, significant hypermetabolism is shown in red across location and corresponding numbered coronal plates in the following regions: OB (1,2), PFC (2), M1/M2 (3,5,6), Cg/RSC (3,5, 7-9), CPu (4,5), IC (4), Pir (4), V1/V2 (7-9), S1/S2 (5,6), cc (5,8), cg (6-8), GP (6), IntC (6) Tha1N (6-8), HPC (7,8), Au1/AuV (7), Pretectal Nucleus (8), PAG (8,9), Midbrain Nuclei (8-10), Colliculi (9-10), Cerebellar Cortex (11), Cerebellar Nuclei (10-12). Hypometabolism clusters are shown in blue and were fewer and smaller in size. In Cntnap2^{-/-} mice, hypermetabolism clusters are located in the following regions: OB (1,2), PFC (2), M1/M2 (3,4), Cg/RSC (3-7), PRhC (5), Pir (5), V1/V2 (6,7), S1/S2 (3-5), Tha1N (5,6), Amyg (5), Hb (5), HPC (6,7), PAG (6), Midbrain Nuclei (6,7), Colliculi (6,7), Cerebellar Cortex (8), Cerebellar Nuclei (7,9).

FIG. 16A-D. Abnormal neuronal network activity and plasticity in Cntnap2 mutant mice. (A) Electrophysiological changes in pyramidal cells at the frontal cortex in Cntnap2^{-/-} mice. Intrinsic electrophysiological analysis of Cntnap2^{-/-} pyramidal cells (n=8) shows a depolarized voltage threshold compared to WT cells (n=9) (units on ordinate are mV and bar shows 5 ms). (B) Example traces of spontaneous excitatory and inhibitory postsynaptic currents (sEPSCs and sIPSCs; left panels) together with cumulative distribution plots for all events recorded from WT (n=18 cells for sEPSCs and n=6 cells for sIPSCs, black) and Cntnap2^{-/-} cells (n=13 cells for sEPSCs and n=7 cells for sIPSCs, red) from pyramidal cells of cortical layers II-III and V (right panels). The analysis shows a reduction in the frequency, but increase in the amplitude of sEPSCs in Cntnap2^{-/-} cells. An increase is also observed in the amplitude of sIPSCs, but without a significant change in the frequency (see box plots under curves). (C) Abnormal spine dynamics in layer V of the frontal association cortex of Cntnap2 mutant

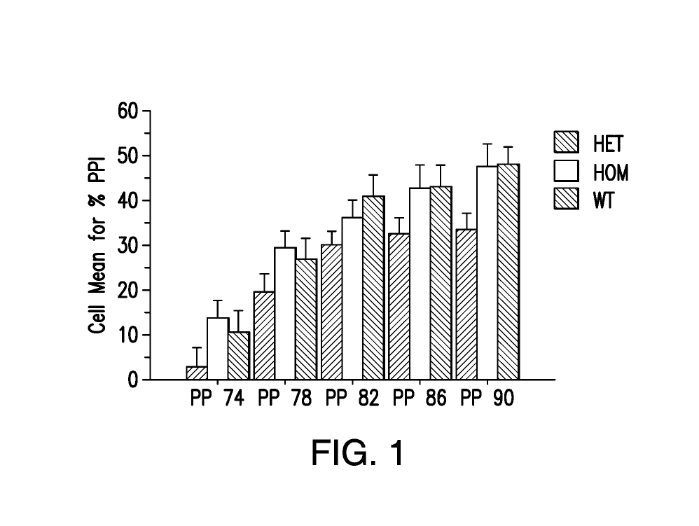

mice. Cntnap2^{+/-}/Thy1-YFP/H and Cntnap2^{-/-}/Thy-1-YFP/H show an increased elimination of spines between P30 and P32 whereas Cntnap2^{+/-}/Thy1-YFP/H mice also demonstrate a decrease in spine formation during this developmental window. Scalebar represents 4 microns. Open arrowheads indicate eliminated spines whereas closed arrowheads indicate newly formed spines; an asterix indicates the presence of filopodia. (D) Bar graphs presenting the results photographically depicted in (C) illustrate the quantified alterations in cortical spine dynamics in Cntnap2 mutant mice. Bars represent, left to right, wild type, Cntnap^{+/-} and Cntnap2^{-/-}. Ordinates present percentages.

FIG. 17A-F. Cellular, synaptic and molecular abnormalities in Cntnap2 mutant mice. (A) Enlarged pyramidal cells in wild type (WT) and Cntnap2 mutants. Left panel shows YFP fluorescence of pyramidal cells in layer V of the frontal cortex of 2-month-old WT (upper image), Cntnap2^{-/-} (middle image), and Cntnap2^{+/-} mice (right image). There is an increased pyramidal soma size in both Cntnap2^{+/-} and Cntnap2^{-/-} mice compared to WT littermates (at 200× magnification). (B and C) Show Cntnap2 dosage effect on pyramidal cell soma size (B) (Cntnap2^{-/-} (“(HM”)>Cntnap2^{+/-} (“HT”)>WT). Cumulative frequency distribution plots (C) show individual pyramidal cell measurements ordered from smallest to largest cell size within each genotype. Significant variability in cell size can be observed in all 3 genotypes. (D) Electron microscopic image demonstrates a perforated postsynaptic density (PSD) in layer I of the cortex from a Cntnap2^{-/-} mouse. (E) Bar graph depicts that the mean length of the segmented PSDs (shown by brackets in (D)) of perforated synapses (arrow points to the perforationin this example) is reduced in layer I of the cortex of Cntnap2^{-/-} mice (micrograph was taken at a magnification of 60000×). (F) Immunoblot analysis to quantify the expression levels of phosphorylated ribosomal protein S6 (pS6), a marker of mTOR activity, in PFC homogenates from 3-5 months and 9-11 months

old Cntnap2 mutant mice and WT littermates. The younger Cntnap2−/− mice demonstrated significantly higher pS6 levels compared with WT littermates, which increased further with age. Bars represent, left to right, wild type, Cntnap^{+/-} and Cntnap2^{-/-}.

FIG. 18A-B. Differential expression of glutamate receptor subunits in the prefrontal cortex (PFC) of Cntnap2 mutant mice. (A) Immunoblot analysis of PFC homogenates from, left to right, WT, Cntnap2^{+/-} and Cntnap2^{-/-} adult mice at two different ages: 3-5 months and 9-11 months. (B) Bar graphs showing results of immunoblots, where bars represent, left to right, wild type, Cntnap^{+/-} and Cntnap2^{-/-}. Indicated are genotype and age-specific changes in the expression profile of ionotropic glutamate receptor (iGluR) and Group I metabotropic glutamate receptors (mGluRs) subunits.

FIG. 19A-C. Cellular, synaptic, and molecular changes in cortical tissue of individuals carrying homozygous CNTNAP2 mutations.

(A) Cresyl violet (CV; blue) staining demonstrates enlarged pyramidal cells throughout the cortex of a patient with homozygous CNTNAP2 mutations (upper left panel) whereas double staining for CV and pS6 (blue and brown, respectively; upper middle panel) shows that pyramidal cells are strongly positive for pS6 compared with a normal control with only minimal pS6 staining (upper right panel). Lower panel shows a representative section from a patient with homozygous CNTNAP2 mutations double labeled with neuronal markers nonphosphorylated neurofilament SMI311 (red), glial marker vimentin (green) and nuclear stain DAPI (blue) Undifferentiated binucleate enlarged cells can be seen which are immunopositive for both SMI311 and vimentin, whereas differentiated neurons are only immunopositive for SMI311. (B) Example of an electron microscope (EM) image of a perforated postsynaptic density (PSD) from an individual with homozygous CNTNAP2 mutations (upper panel). The presynaptic
membrane with clusters of vesicles, the synaptic cleft, and postsynaptic membrane with active zone can be seen. Bar graph indicates a reduction in length in perforated PSDs in homozygous mutation carriers versus controls (bottom panel).
(C) Staining for MAP-2 (red) and GluR1 (green; left panel) and mGluR5 (green; right panel) demonstrates an increase of both glutamate receptor subunits in pyramidal cells throughout the cortex of patients with homozygous CNTNAP2 mutations. Cell nuclei are visualized with DAPI stain (blue). Scale bars in A and C represent 20 microns.

FIG. 20A-E. Treatment with WYE125132 rescues cellular, synaptic and molecular abnormalities in Cntnap2 mutants. (A) Reversal of increased cell size by treatment with WYE125132. Panels demonstrate pyramidal cells throughout the cortex of both Cntnap2^{+/-} (HT) and Cntnap2^{-/-} (HM) mice compared with WT littermates, which is entirely rescued with treatment with WYE125132 or vehicle. (B) Bar graphs (top) indicate that the dramatic increase in phosphorylation of S6 (Phospho-S6: S6) in the cortex of Cntnap2−/− and Cntnap2+/− mice is completely reversed with by treatment with WYE125132. In Bar graphs, each pair reflects untreated (left) and treated (right) values. Representative immunoblots are shown (bottom). (C) Distribution diagram showing soma size (pixels) for, left to right, untreated wildtype, untreated Cntnap2^{-/-} (HM), treated wildtype and treated Cntnap2^{-/-} (HM) (ordinate shows 0-5000 pixels). (D) Relative frequency versus soma size (pixels) for curves showing treated (“tr”) versus untreated (“utr”) wildtype and Cntnap2^{-/-} (HM). (E) Treatment with WYE125132 leads to a complete normalization of all glutamate receptor subunits GluR1, GluR2, NR1, NR2A, NR2B and mGluR5. Representative immunoblots are shown (bottom right).

FIG. 21A-E. Treatment with WYE125132 rescues abnormalities in synaptic plasticity and the excitatory-inhibitory balance as well as alterations in dendritic spine dynamics and behavioral deficits in Cntnap2 mutants.

(A) Representative recordings showing measurements of excitation (“E”) and inhibition (“I”) at different stimulus intensities (normalized to maximum intensity of 15 V) from WT vehicle-treated (top; linear correlation coefficient r: 0.79), Cntnap2^{-/-} vehicle-treated (middle; r: 0.07), and WYE125132-treated Cntnap2^{-/-} mice (bottom; r: 0.72) mice. (B) Example whole-cell recordings from layer V pyramidal neurons of adult auditory cortex from WT vehicle-treated (upper; synaptic modification: 50.3% increase), vehicle-treated Cntnap2^{-/-} (middle; synaptic modification: −13.6% decrease), and WYE125132-treated Cntnap2^{-/-} mice (lower; synaptic modification: 35.1% increase) mice. Red line represents
average synaptic strength recorded 6-15 minutes after spike pairing ended (at time 0). Summary of LTP experiments are shown in bar graphs (lower panel). WYE125132-treated Cntnap2^{-/-} mice, synaptic modification: 45.4±16.4% increase, n=8; vehicle-treated Cntnap2^{-/-}, synaptic modification: −4.4±13.3% decrease, n=9, p<0.03 compared to WYE125132-treated Cntnap2^{-/-} mice, Student's two-tailed t-test; WT animals, synaptic modification: 33.8±11.4% increase, n=6. Summary of excitatory-inhibitory correlation measurements are shown in bar graphs (lower panel). WYE125132-treated Cntnap2−/−, r: 0.43±0.08, n=12; vehicle-treated
Cntnap2^{-/-}, r: 0.09±0.13, n=17, p<0.04 compared to WYE125132-treated Cntnap2^{-/-} mice Student's two-tailed t-test; WT animals, r: 0.59±0.06, n=9. Error bars represent s.e.m. (C) Correction of abnormalities in spine dynamics. Treatment with WYE125132 rescues excessive spine elimination in both Cntnap2^{+/-} and Cntnap2^{-/-} mice. (D) Rescue of social interaction deficits. Treatment with WYE125132 corrects the social interaction deficits in the first minute of testing as well as the total frequency of social interactions over the whole 10-minute testing interval in Cntnap2 mutant mice. (E) Rescue of cognitive deficits. WYE125132-treated mutant mice demonstrate a rescue of a deficit in the Novel Object Recognition test.

FIG. 22. Western blot of cortical extracts from Cntnap2 mutant or wild-type mice treated with vehicle or various mTOR pathway inhibitors, showing staining for the presence of phosphorylated S6. Individual mice were tested, and are represented in the lanes as follows. Lane 1=Cntnap2^{+/-} mouse ˜9-10 weeks old treated with vehicle as for AZD2014; Lane 2=Cntnap2^{+/-} mouse ˜7.3 months old treated with rapamycin; Lane 3=Cntnap2^{+/-} mouse ˜6.3 months old treated with Torin 2; Lane 4=Cntnap^{-/-} mouse ˜9-10 weeks old treated with AZD2014; Lane 5=wild-type mouse ˜4 months old treated with vehicle as for Torin2; Lane 6=Cntnap2^{+/-} mouse 9-10 weeks old treated with vehicle as for AZD2014; Lane 7=Cntnap2^{+/-} mouse ˜5 months old treated with rapamycin; Lane 8=Cntnap2^{-/-} mouse ˜5 months old treated with AZD2014; and Lane 9=wild-type mouse ˜4 months old treated with vehicle as for Torin 2.

### 5. DETAILED DESCRIPTION OF THE INVENTION

The present invention relates to methods of treating neurodevelopmental and/or neuropsychiatric disorders comprising administering, to a subject in need of such treatment, a therapeutically effective amount of an inhibitor of the mTOR pathway, as described herein. A subject may be a human subject or a non-human subject such as, but not limited to, a non-human primate, a dog, a cat, a horse, a pig, a cow, a sheep, a goat, a mouse, a rat, a hamster, a guinea pig, fowl, a cetacean, etc.

Without limitation, examples of neurodevelopmental and/or neuropsychiatric disorders which may be treated include, but are not limited to, schizophrenia (SCZ), autism spectrum disorder (ASD) (such as, for example, but not limited to, autistic disorder, Asperger's syndrome, pervasive developmental disorder not otherwise specified, Rett's syndrome, childhood disintegrative disorder), bipolar disorder, attention-deficit hyperactivity disorder (ADHD), Gilles de la Tourette disorder, obsessive-compulsive disorder, depression, mood disorders, seizure disorder, cognitive dysfunction and/or mental retardation. Treatment is achieved when a subject exhibits improvement in a symptom or sign of the disorder. As a non-limiting example, treatment may be reflected by an improvement in the Global Assessment of Functioning score of the subject, for example, but not by way of limitation, which is sustained over a period of at least one month, at least 3 months, at least six months, or at least one year. In non-limiting embodiments, where the disorder is an autism spectrum disorder, it is not associated with a genetic defect in TSC, FXS, or PTEN and/or an associated clinical syndrome, e.g. tuberous sclerosis or Fragile X syndrome.

Non-limiting examples of mTOR pathway inhibitors which may be used according to various embodiments of the invention include everolimus, ridaforolimus, ARmTOR26 (Array BioPharma Inc.), BN107 (Bionovo, Inc.), CU906 (Curis, Inc.), ECO565 (Endocyte, Inc.), XL388 (Exelixis Inc.), HL152B (HanAli Biopharma Co. Ltd.), NV128 (MEI Pharma Inc., Novogen, Ltd.), sirolimus, SXMTR1 (Serometrix L.L.C.), X480 (Xcovery), X414 (Xcovery), INK128 (Takeda Pharmaceutical Co. Ltd.), SAR245409 (Sanofi), XL765 (Sanofi), P7170 (Piramal Enterprises), ME344 (Novogen Ltd.), BEZ235 (Novartis), temsirolimus, GDC0980 (Genentech, Inc.), MLN0128 (Millennium Pharmaceuticals), RG7422 (F. Hoffman La Roche Ltd.), DS3078 (Daiichi Sankyo Co. Ltd.), OS1027 (Astellas Pharma US Inc.), AZD2014 (AstraZeneca Plc), AZD8055 (Astrazeneca Plc.), GDC0068 (Array BioPharma Inc.), CC223 (Celgene Corp.), CC115 (Celgene Corp.), zotarolimus, umirolimus (Terumo Corp.), tacrolimus, TOP216 (Topotarget AS), BC210 (Pfizer Inc.), PF04691502 (Pfizer Inc.), WYE125132 (Pfizer Inc.), TAFA93 (Isotechnika Pharma Inc.), LOR220 (Lorus Therapeutics Inc.), nPT-mTOR (Biotica Technology), AP23841 (ARIAD Pharmaceuticals Inc.), AP24170 (ARIAD Pharmaceuticals Inc.), and Torin2 (Tocris).

In certain non-limiting embodiments of the invention, the mTOR pathway inhibitor is rapamycin or a rapamycin analog (“rapalog”). In alternate embodiments of the invention, the mTOR pathway inhibitor is not rapamycin or a rapamycin analog; for example, but not by way of limitation, the mTOR pathway inhibitor may be a so-called mTOR kinase inhibitor such as an ATP-competitive inhibitor of mTOR kinase (see Yu et al., “Beyond Rapalog Therapy: Preclinical pharmacology and antitumor activity of WYE-125132, an ATP-competitive and specific inhibitor of mTORC1 and mTORC2,” Cancer Res. 70(2):621-631 (2010); Shor et al., “Requirement of the mTOR kinase for the regulation of Maf1 phosphorylation and control of RNA polymerase III-dependent transcription in cancer cells,” J Biol Chem. 285(20):1538015392 (2010); Yu and Toral-Barza, “Biochemical and pharmacological inhibition of mTOR by rapamycin and an ATP-competitive mTOR inhibitor”, Chapter 2 in Weichart, ed. “mTOR: Methods and Protocols, Methods in Molecular Biology vol 821, pp. 15-26 (2012); Pike et al., “Optimization of potent and selective dual mTORC1 and mtORC2 inhibitors: the discovery of AZD8055 and AZD2014,” Bioorg. Med. Chem. Lett. 23:1212-1216 (2013), where the disclosures and compounds referred to in these references are incorporated by reference herein).

In particular non-limiting embodiments, the mTOR kinase inhibitor is a pyrazolopyrimidine ATP-competitor and specific inhibitor of mTORC1 and/or mTORC2. In particular non-limiting embodiments, the mTOR kinase inhibitor is a pyrazolopyrimidine substituted with a bridged morpholine ATP-competitor and specific inhibitor of mTORC1 and/or mTORC2. In a specific non-limiting embodiment, the mTOR pathway inhibitor is WYE-125132 (also sometimes referred to as “WYE-132”) or an analog thereof. The structure of WYE-125132 is:

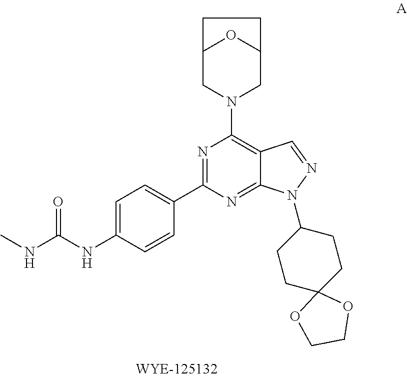 [SMILES: CNC(=O)Nc1ccc(-c2nc(N3CC4CCC(C3)O4)c3cnn(C4CCC5(CC4)OCCO5)c3n2)cc1]

In particular non-limiting embodiments, the mTOR kinase inhibitor has the general formula I:

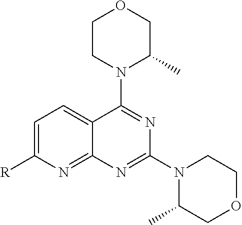

where R is a substituted or unsubstituted aromatic, for example a substituted or unsubstituted phenyl, where when present the one or more substituent may be, independently, a halogen such as fluorine, chlorine or bromine, a hydroxyl, a C₁-C₄ alkoxy, or a substituted or unsubstituted amide where a substituent may be, for example, C₁-C₄ alkyl. In non-limiting embodiments, R may be

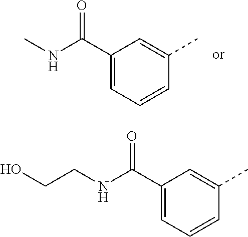

(where the former is AZD2014; see Pike et al.).

Additional non-limiting examples of mTOR kinase inhibitors which may be used according to the invention include compounds disclosed in US20110281857, EP2382207, US20120165334, EP2398791, US20090311217, EP2300460, US20120134959, and EP2419432.

In particular non-limiting embodiments, the mTOR kinase inhibitor has the general formula II:

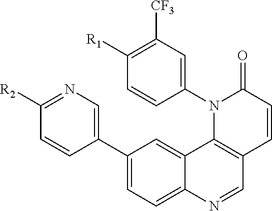

where R₁ may be H, or C₁-C₄ alkyl, or substituted or unsubstituted amino, or a 3-6 member aliphatic or aromatic ring, which may optionally be a heterocycle comprising at least one N where said ring may be substituted or unsubstituted, where when present the one or more substituent may be, independently, a halogen such as fluorine, chlorine or bromine, a hydroxyl, a C₁-C₄ alkoxy, or a substituted or unsubstituted amide; and where R₂ is a substituted or unsubstituted amine, a halogen such as fluorine, chlorine or bromine, a hydroxyl, or a C₁-C₄ alkoxy, where a substituent may be, for example, C₁-C₄ alkyl. In a specific non-limiting embodiment, the specific compound having general formula II is Torin 2, having the structure:

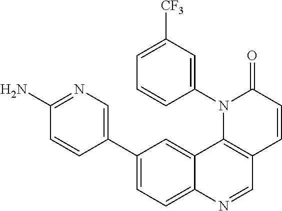

In non-limiting embodiments, an effective dose of a pyrazolopyrimidine substituted with a bridged morpholine ATP-competitor and specific inhibitor of mTORC1 and/or mTORC2, of which WYE-125132 is a non-limiting example, may be, for treatment of a human subject, between 0.5 and 100 mg/kg, or between about 1 and 50 mg/kg, or between about 5 and 25 mg/kg, or about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/k, about 24 mg/kg, or about 25 mg/kg.

In non-limiting embodiments, an effective dose of AZD2014 or a related compound having general formula I may be, for treatment of a human subject, between about 0.2 and 5 mg/kg, or between about 0.5 and 2 mg/kg, or between about 1 and 2 mg/kg, or greater than 1 mg/kg and less than 3 mg/kg, or about 0.5 mg/kg, or about 0.75 mg/kg, or about 1 mg/kg, or about 1.25 mg/kg, or about 1.5 mg/kg, or about 1.75 mg/kg, or about 2 mg/kg.

In non-limiting embodiments, an effective dose of Torin2 or a related compound having general formula II may be, for treatment of a human subject, between about 0.2 and 5 mg/kg, or between about 0.5 and 2 mg/kg, or between about 1 and 2 mg/kg, or greater than 1 mg/kg and less than 3 mg/kg, or about 0.5 mg/kg, or about 0.75 mg/kg, or about 1 mg/kg, or about 1.25 mg/kg, or about 1.5 mg/kg, or about 1.75 mg/kg, or about 2 mg/kg.

An effective dose of other mTOR pathway inhibitors, for example rapalogs or non-rapalog mTOR kinase inhibitors, may be a dose calculated to achieve a concentration in the central nervous system of a subject to be treated, where said concentration, in cell culture, inhibits intracellular phosphorylation of S6K relative to untreated cells, preferably by at least about 20 percent.

The mTOR pathway inhibitor may be administered according to methods known in the art, including but not limited to oral, sublingual, nasal, by inhalation, transdermal, subcutaneous, intradermal, intramuscular, intravenous, intraperitoneal, intrathecal, etc. In certain non-limiting embodiments, a pyrazolopyrimidine substituted with a bridged morpholine ATP-competitor and specific inhibitor of mTORC1 and/or mTORC2, of which WYE-125132 is a non-limiting example, may be administered orally.

In certain embodiments, the inhibitor of the mTOR pathway is administered to a subject in an amount effective to decrease the level of S6 phosphorylation.

In certain embodiments, the inhibitor of the mTOR pathway is administered to a subject in an amount effective to increase expression of a glutamate receptor subunit, for example, a GluR2 receptor subunit, an NR2A receptor subunit, or combinations thereof.

In certain embodiments, the inhibitor of the mTOR pathway is administered to a subject in an amount effective to decrease expression of a glutamate receptor subunit, for example, a GluR1 receptor subunit, an NR1 receptor subunit, an NR2B receptor subunit, an mGLUR1 receptor subunit, an mGLUR5 receptor subunit, or combinations thereof.

In certain embodiments, the inhibitor of the mTOR pathway is administered to a subject in an amount effective to increase social interaction and/or cognition.

In certain embodiments, the inhibitor of the mTOR pathway is administered to a subject in an amount effective to decrease the subject's susceptibility to seizures. In certain embodiments, the inhibitor of the mTOR pathway is administered to a subject in an amount effective to increase the subject's threshold for seizures, for example, increasing the subject's threshold to a seizure stimulant such as, for example, pilocarpine.

In a specific, non-limiting embodiment of the invention, a subject suffering from a neurodevelopmental and/or neuropsychiatric disorder may be tested to determine whether a copy number variation or other mutation or variation in CNTNAP2 is present, and if a copy number variation, mutation or variation in CNTNAP2 is found, then the subject may be treated with a mTOR pathway inhibitor. Such a test may be performed using methods known in the art, including but not limited to nucleic acid based testing, for example, using nucleic acid primers followed by amplification and sequencing, and/or microarray analysis, SNP analysis, use of nucleic acid probes, for example in FISH analysis, etc., or protein based testing such as antibody based analysis, Western blotting, etc. In non-limiting embodiments the present invention provides for kits for making such determination.

In related non-limiting embodiments a subject suffering from a neurodevelopmental and/or neuropsychiatric disorder may be tested to determine whether the subject exhibits a hyperactivation in the mTOR pathway, and if hyperactivation of the mTOR pathway is found, then the subject may be treated with a mTOR pathway inhibitor. Indicators of hyperactivation of the mTOR pathway include, but are not limited to, for example, as demonstrated in a cell sample from the subject: increased phosphorylation of S6, decreased GluR2 receptor subunit, decreased NR2A receptor subunit, increased GluR1 receptor subunit, increased NR1 receptor subunit, increased NR2B receptor subunit, increased mGLUR1 receptor subunit, and/or increased mGLUR5 receptor subunit, relative to a normal control subject. Such a test may be performed using methods known in the art, including but not limited to nucleic acid based testing, for example, using nucleic acid primers followed by amplification and sequencing, and/or microarray analysis, SNP analysis, use of nucleic acid probes, for example in FISH analysis, etc., or protein based testing such as antibody based analysis, Western blotting, etc. In certain non-limiting embodiments the testing may be performed in vivo, for example using PET scanning in conjunction with administration of a known mTOR inhibitor. In one specific non-limiting embodiment the level of activation of the mTOR pathway may be assessed in vitro via phosphorylation of S6, as described in the working examples below. In non-limiting embodiments the present invention provides for kits for making such determination.

Treatment of a subject with a mTOR pathway inhibitor may be practiced in conjunction with one or more conventional treatments of the neurodevelopmental or neuropsychiatric disease being treated.

### 6. EXAMPLE

### Lack of Caspr2 Leads to a Lower Seizure Threshold and Behavioral Abnormalities Related to SCZ and ASD

For assessment of the seizure threshold in the Caspr2 mutant mice and wild-type littermates, we utilized a pilocarpine seizure threshold paradigm in 7 wild-type littermates (WT), 9 Caspr2^{+/-} mice (HET), and 7 Caspr2^{-/-} (HOM) mice. We found a positive association between homozygosity for the mutant allele and reaching the higher stages of the seizure phenotype (P=0.027) and a strong trend in mice heterozygous for the mutant allele, indicating that lack of Caspr2 leads to a lowering of the seizure threshold in mice. We then performed a behavioral characterization of a separate cohort of Caspr2 mutant mice and WT littermates, focusing on sensorimotor gating—as measure by prepulse inhibition (PPI)—as well as performing an assessment of the mutant mice in the Open Field Test paradigm. We utilized 20 WT littermates, 19 Caspr2^{+/-} HET mice, and 17 Caspr2^{-/-} HOM mice and were able to demonstrate a significant reduction in PPI in Caspr2^{+/-} HETs compared to WT littermates (P=0.0384) as well as Caspr2^{-/-} HOM mice (P=0.0399).

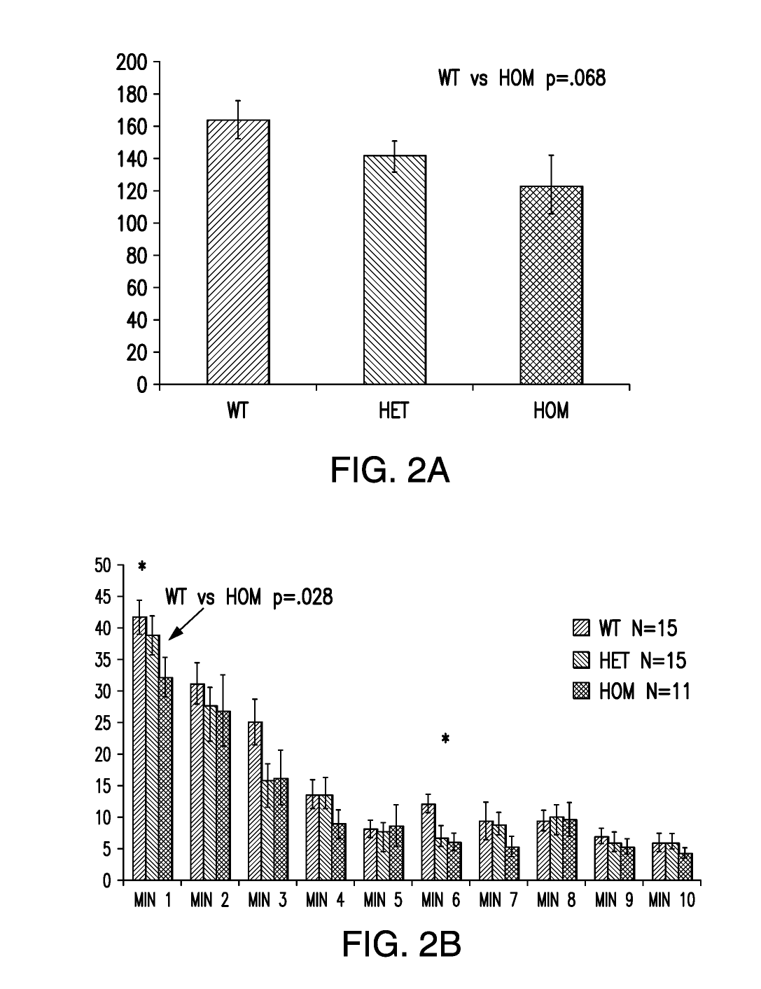

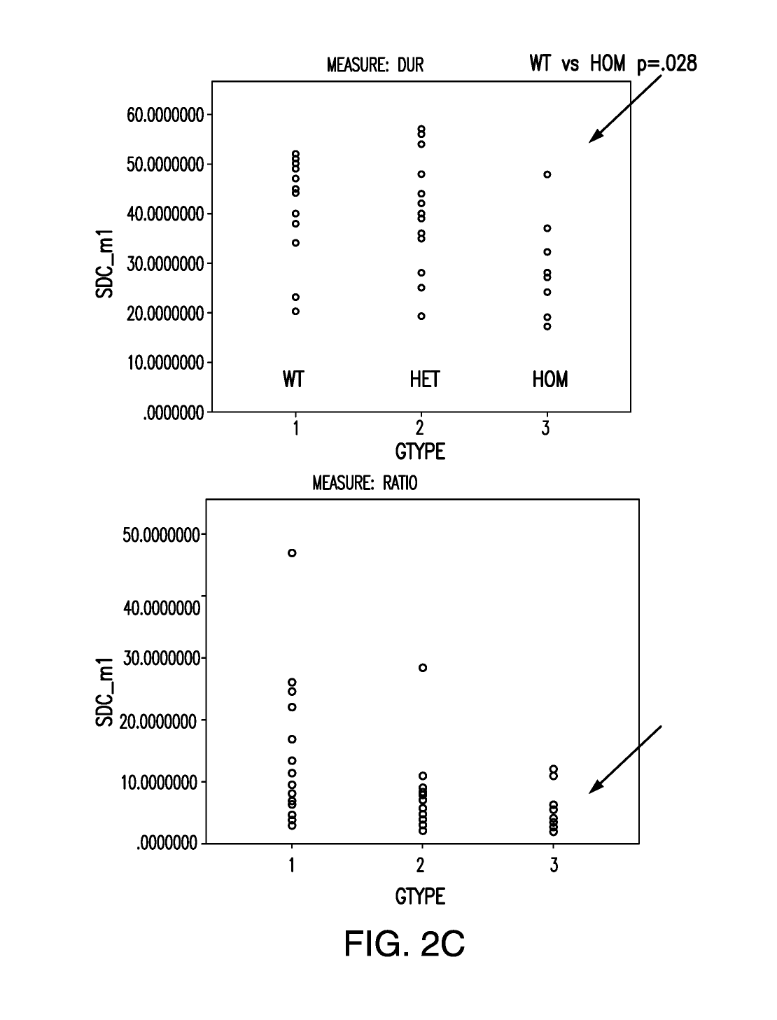

Furthermore, we assessed Caspr2 mutant mice at postnatal day (P) 60 to investigate ASD-related behaviors, utilizing a social interaction paradigm. We analyzed data from a total of 15 WT, 15 Caspr2 HET mice, and 11 Caspr2^{-/-} HOM mice and were able to demonstrate a statistically significant reduction of social interaction in Caspr2^{-/-} HOM mice during several of the first minute intervals of the 10-minute assessment (see FIG. 2B). This is indicative of a significant impairment in social interaction in the Caspr2 mutant mice. This is also a replication of a recent study, which reported similar ASD-related behavioral abnormalities in the Caspr2 mice (Penigarikano et al., 2011).

Finally, we also found that Caspr2^{-/-} mice have on average statistically significantly shorter bouts of sniffing compared to wild-type littermates during the first minute of the social interaction paradigm (see FIG. 2C). This is thought to be indicative of anxiety surrounding social novelty, and is also potentially consistent with an ASDphenotype in Caspr2 mutant mice.

Lack of Caspr2 Leads to an Abnormal Cellular Phenotype and Overactivation of the mTOR Pathway.

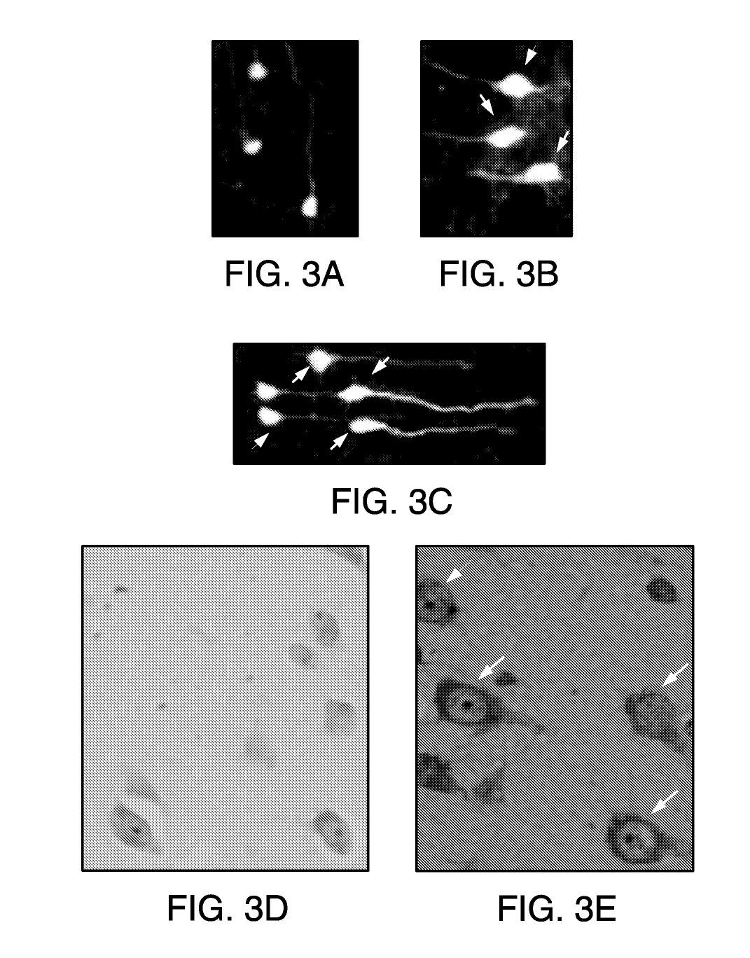

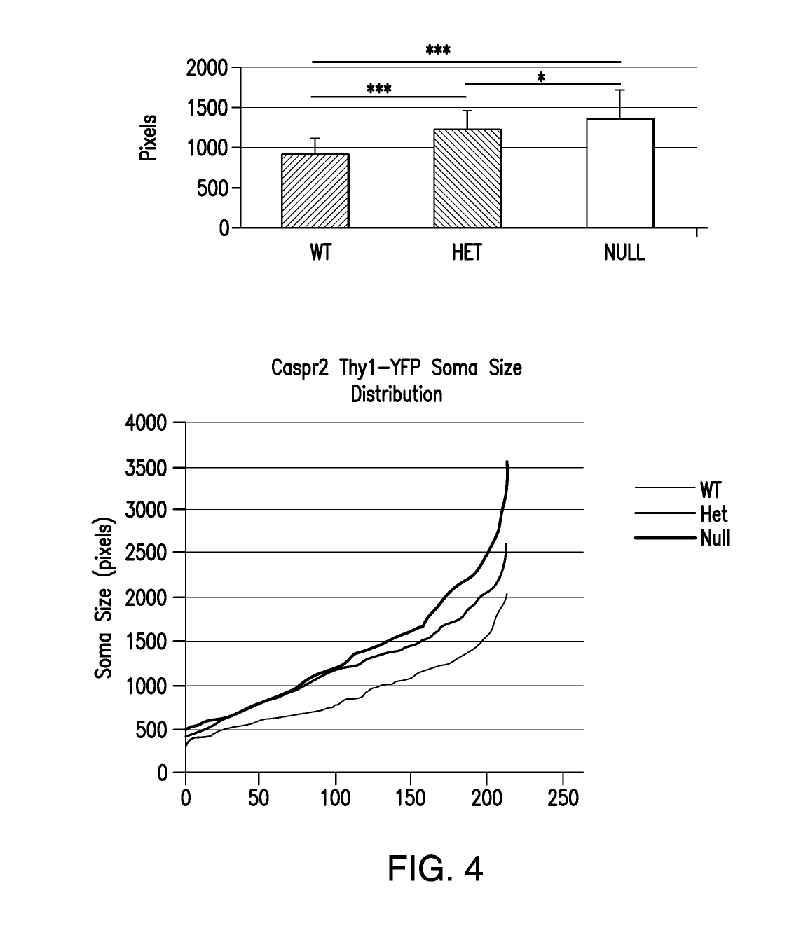

Coronal sections of Caspr2/Thy1-YFP/H mice were analyzed at 200× magnification utilizing epifluorescent microscope analysis of pyramidal cells in the frontal cortex (FC). Soma size was traced on Photoshop to quantify pixels. At the cellular level, Caspr2^{+/-}/Thy1-YFP/H and Caspr2^{-/-}/Thy-1-YFP/H mice were found to have pyramidal cells with a significantly enlarged soma size throughout layer 5 of the FC and temporal cortex (TC)(FIG. 3A, B). We also found an enlarged soma size of pyramidal cells in the CA1 region of the hippocampus (HPC), suggesting that this is a robust and diffuse cellular phenotype associated with the Caspr2 null mutation. In addition to the increased soma size phenotype, using several cortical markers (i.e., Cux1, DAPI), we were also able to demonstrate an abnormal cortical organization in the Caspr2 mutant mice compared to WT littermates, indicative of abnormal neuronal cell migration. Cresyl violet staining of TC tissue from human Old-Order Amish subjects with homozygous frameshift mutations within the CNTNAP2 gene, also identified similarly abnormal pyramidal cells throughout all layers of the TC with an enlarged soma size (see FIG. 3E) as well as a comparable pattern of abnormal overall cortical architecture. A similar phenotype of enlarged pyramidal cells has previously been described in different transgenic mouse models of ASD which all involve overactivation of the mTOR pathway, including models of tuberous sclerosis complex (Goto et al., 2011) and PTEN mutations (Zhou et al., 2009).

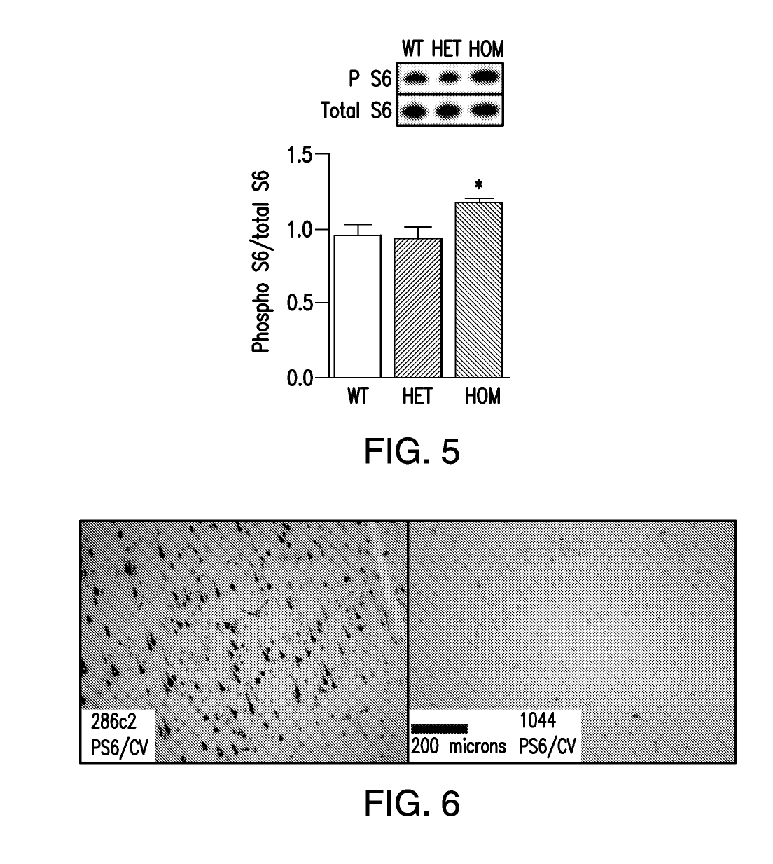

Western blot analysis of the FC, TC, and HPC of Caspr2^{+/-}, Caspr2^{-/-} and WT littermates, clearly demonstrated a statistically significant ˜20% increase in phosphorylated S6 in Caspr2^{-/-} HOM mutant mice compared to total S6 in these regions (FIG. 5; P<0.05). This is the first report of an effect of Caspr2 deficiency on mTOR signaling and it is consistent with our findings of increased pyramidal cell size in both the mouse model and the human brain tissue of patients with the CNTNAP2 mutation since phosphorylation of S6 is directly associated with regulating cell size (Ruvinsky et al., 2006).

To confirm this finding from the Caspr2 mouse model, we subsequently examined TC tissue from Old-Order Amish subjects who carry a homozygous mutation of CNTNAP2 and suffer from ASD. When performing a co-staining with cresyl violet and using an antibody directed against pS6, the brain tissue of eight age- and gender-matched normal controls demonstrated normal-sized pyramidal cells with no significant staining for pS6, whereas the brain tissue from the three Old-Order Amish patients demonstrated a dramatic staining for pS6 in the enlarged pyramidal cells throughout TC (FIG. 6). Together, these findings suggest that the mutation of CNTNAP2 leads to overactivation of the mTOR pathway (as measured by pS6), which, in turn, leads to cellular abnormalities (i.e., increased size of pyramidal cells) and neurocircuitry level deficits (i.e., abnormalities in excitation-inhibition balance, see below). It is therefore very likely that, by effectively targeting the mTOR overactivation in cells that

carry a CNTNAP2 mutation, one might be able to prevent and/or reverse ASD-associated changes in cellular morphology and electrophysiology.

Lack of Caspr2 Leads to Disturbance of the Excitation/Inhibition Balance in the Frontal Cortex.

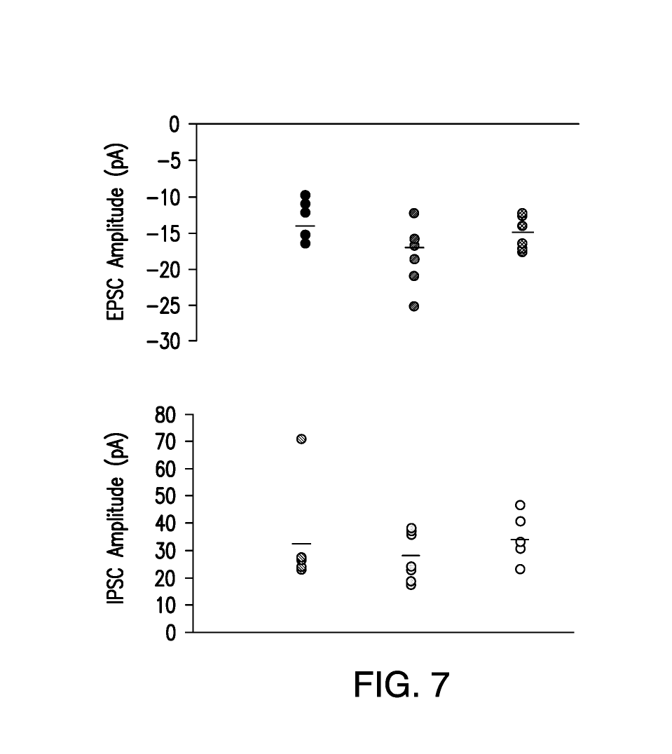

We performed electrophysiological studies to examine the balance between excitation and inhibition in the FC of Caspr2 mutant mice aged P21-P28. Our analysis revealed a significant increase in the excitatory postsynaptic current (EPSC) amplitude in Caspr2^{-/-}/Thy-1-YFP/H mice compared to WT littermates (FIG. 7). The Caspr2^{+/-}/Thy1-YFP/H mice also showed a trend toward increased EPSC amplitude, but this did not reach statistical significance. There was no difference in either the amplitude or the frequency of the inhibitory postsynaptic current (IPSC) of Caspr2^{-/-}/Thy-1-YFP/H or Caspr2^{+/-}/Thy1-YFP/H compared to WT littermates. Overall, this data demonstrates a shift from a balance between inhibition and excitation towards more excitation within the neural microcircuitry of the developing brain of the Caspr2 mutant mice.

Lack of Caspr2 Leads to Abnormalities in Cortical Spine Dynamics.

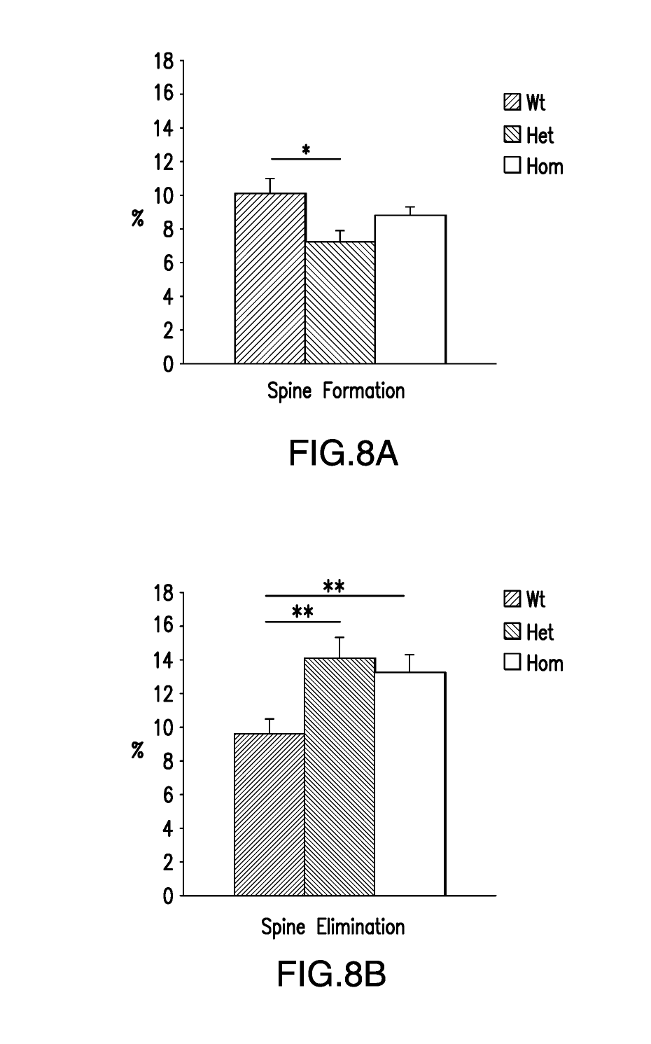

Using transcranial two-photon microscopy, we also studied the baseline rate of dendritic spine formation and elimination in the intact, living mouse brain at P30 and P32. We have succeeded in visualizing the in vivo spine dynamics in FC layer 5 in seven Caspr2^{+/-}/Thy1-YFP/H and seven Caspr2^{-/-}/Thy-1-YFP/H mice, compared to seven WT littermates also positive for YFP/H. We saw a significantly lower rate of dendritic spine formation in the Caspr2^{+/-}/Thy1-YFP/H mice (P=0.006) and a nearly significant decrease in the Caspr2^{-/-}/Thy1-YFP/H mice (P=0.07), compared to WT littermates (FIG. 8A). Furthermore, both Caspr2^{+/+}/Thy1-YFP/H and Caspr2^{-/-}/Thy-1-YFP/H mice were found to have an even more significantly increased spine elimination at P30 compared to the control mice (P=0.007 and P=0.003, respectively; FIG. 8B), suggesting that Caspr2 mutant mice undergo excessive synaptic pruning, which could ultimately lead to decreased cortical connectivity.

Lack of Caspr2 Leads to Abnormities in Synaptic Structure.

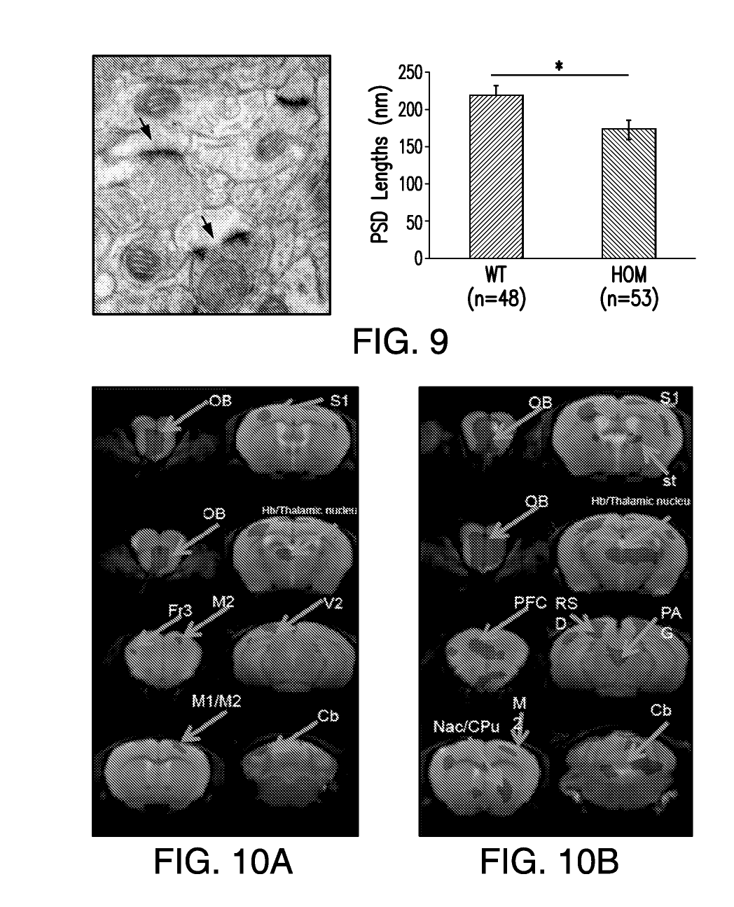

Finally, we carried out electron microscopy analysis of the synaptic structure in FC layer 5 in mutant mice and WT littermates. We found that the length of the perforated post-synaptic density (PSD) in Caspr2^{-/-} HOM mice is reduced by ˜25% (FIG. 9), a finding that has also been reported in various animal models of cognitive deficits (Nicholson et al., 2004). Formation of perforated PSDs in axospinous synapses has been demonstrated to be the structural correlate of an enhanced efficacy of synaptic transmission, which, in turn, is believed to underlie long-term potentiation (LTP) induction and learning (Hering et al., 2001). Therefore, the decrease in length of perforated PSDs may reflect the structural synaptic deficits underlying the previously characterized cognitive deficits, seizures, and the ASD-related behavioral abnormalities in Caspr2 mutant mice and human subjects with CNTNAP2 mutations.

Lack of Caspr2 Leads to Hypermetabolism in Cortical and Subcortical Brain Structures.

We performed baseline FDG Micro-PET-MRI scanning of Caspr2^{+/-}, Caspr2^{-/-}, and WT littermates under anesthesia and initiated PET data acquisition several seconds before i.v. injection of the radiolabeled 2-deoxy-2-[18F]fluoro-D-glucose (FDG) via a tail-vein catheter (Luat et al., 2007). During PET acquisition, anatomic magnetic resonance images were acquired. After scanning, images were reconstructed using the maximum-a-posteriori (MAP) algorithm (10 iterations, 0.01 smoothing value, 256×256 resolution). After reconstruction, the voxel sizes were x=2.0, y=2.0, z=2.0 mm. All images were spatially normalized, and later co-registered to a magnetic resonance image (MM) template of the mouse brain using the PMOD software (PMOD Technologies, Zurich, Switzerland). A template detailing regions of interest (ROI) was made in accordance with the MM used for co-registration to allow for a more simple method of detailing regional brain activation. In addition, Statistical Parametric Mapping (SPM) was used for uPET image analysis. Images from each group were averaged to create a “template image” for the challenge scan and a template image for the follow-up scan. The template images were then smoothed (2 mm Gaussian) and used to spatially normalize each image. Images were then smoothed (2 mm Gaussian) and 6 one-way ANOVAs were run comparing scans between groups (WT to Caspr2^{+/-}; Caspr2^{+/-} to WT; WT to Caspr2^{-/-}; Caspr2^{-/-} to WT; Caspr2^{+/-} to Caspr2^{-/-}; Caspr2^{-/-} to Caspr2^{+/-}).

Statistical Parametric Mapping (SPM):

All differences in BGluM in all contrasts had a minimum cluster size of 50 voxels and statistical significance was set to α=0.01. Comparisons between Caspr2^{+/-} and WT animals revealed activation in Caspr2^{+/-} group in the olfactory bulb (OB), motor cortex (M2 & M1), prefrontal cortex (PFC), somatosensory cortex (S1), caudate putamen (CPu), nucleus accumbens (NAc), stria terminalis (st), habenula (Hb), thalamic nucleus (Th) visual cortex (V2), retrosplenial cortex (RSC), periaqueductal gray (PAG), cerebellum (Cb) (FIG. 10A). Comparisons between Caspr2^{-/-} and WT animals revealed activation in Caspr2^{-/-} group in the olfactory bulb (OB), motor cortex (M2 & M1), Fr (frontal cortex), somatosensory cortex (S1), habenula (Hb), thalamic nucleus (Th), visual cortex (V2), cerebellum (Cb) (FIG. 10B).

SPM analysis revealed significant activation in the Caspr2^{+/-} group compared to the WT group (P<0.01, Ke>50) in the following regions: OB, M1/M2, PFC, S1, CPu, NAc, st, Hb, Th, V2, RSC, PAG, and Cb. Of these regions, the greatest activation was seen in the OB (spanning from the OB through PFC) and Th (spanning from Th and Hb through PAG) (see FIG. 10A). Results also revealed significant activation in the Caspr2^{-/-} group (P<0.01, Ke>50) compared to the WT group in the following regions: OB, M1/M2, Fr, 51, Th, V2, and Cb. Of these regions, greatest activation was seen in the OB (see FIG. 10B). A qualitative analysis of both contrasts reveals identical patterns of activation in the Caspr2^{+/-} and Caspr2^{-/-} groups compared to the WT group involving the OB, M1/M2, 51, Hb, Th, V2, and Cb, suggesting a possible pathway of activation in response to HET or HOM deletion of Caspr2. Interestingly, previous FDG-PET studies involving human subjects with tuberous sclerosis, another disorder associated with mTOR overactivation and a strong association with ASD, have demonstrated hypermetabolism in certain brain structures (Nicholson et al., 2004). As such, our findings of hypermetabolism in cortical and subcortical brain structures with FDG-PET imaging in Caspr2 mutant mice suggest that this could be utilized as a biomarker of disease activity and treatment response.

Lack of Caspr2 Leads to Decrease in Occipital Lobe Volume on MRL.

Images were acquired on a 7 Tesla Mill scanner with a 40 cm bore diameter. The sequence used wasa T2 weighted 3D fast spin echo (FSE), with a TR of 200 ms, an echo train length of 6, an effective TE of 42 ms, a field of view (FOV) of 25 mm×28 mm×14 mm, and a matrix size of 450×504×250, which leads to an isotropic resolution of 56 μm. In the first phase encode dimension, consecutive kspace lines were acquired with alternating echoes to move discontinuity related ghosting artifacts to the edges of the FOV. The FOV direction was subsequently cropped to 14 mm after reconstruction. Total imaging time for the acquisition was 11.7 hours.

To test for any volumetric changes in the Caspr2^{+/-} and Caspr2^{-/-} mice compared to the WT mice images from the MRI scans were linearly (6 parameter followed by a 12 parameter) and, subsequently, non-linearly registered. All scans were then re-sampled with the appropriate transform and averaged to create a population atlas, which represents the average anatomy of all brains. Registrations were performed with a combination of the mni_autoreg tolls and ANTS. The result of this registration is to have all scans deformed into exact alignment with each other in an unbiased fashion. This allows for the analysis of the deformations needed to take each individual brain into the final atlas space, the goal being to model how the deformation fields relate to genotype. The Jacobian determinants of the deformation fields are then calculated as measurements of volume at each individual voxel. Significant regional volume changes can then be calculated in two different ways. First, regional measurements can be calculated by registering a pre-existing classified MRI atlas on to the population atlas, which allows for the volume measurement of 62 different brain regions. The 62 regions in the classified atlas include the cortical lobes, large white matter structures (i.e., the corpus callosum), ventricles, cerebellum, brain stem structures, and olfactory bulbs. The regions were then assessed in all brains and volumes were calculated in mm3. Second, individual voxel measurements can be calculated from comparisons of the Jacobian determinants in a specific voxel between the Caspr2^{+/-}, Caspr2^{-/-}, and WT mice. These measures can be calculated as measures of absolute volume (in mm3) or relative volume (% total brain volume). Multiple comparisons were controlled for by using either the False Discovery Rate (FDR) for the regional comparisons, or Threshold Free Cluster Enhancement (TFCE) for the voxel-wise whole brain comparisons.

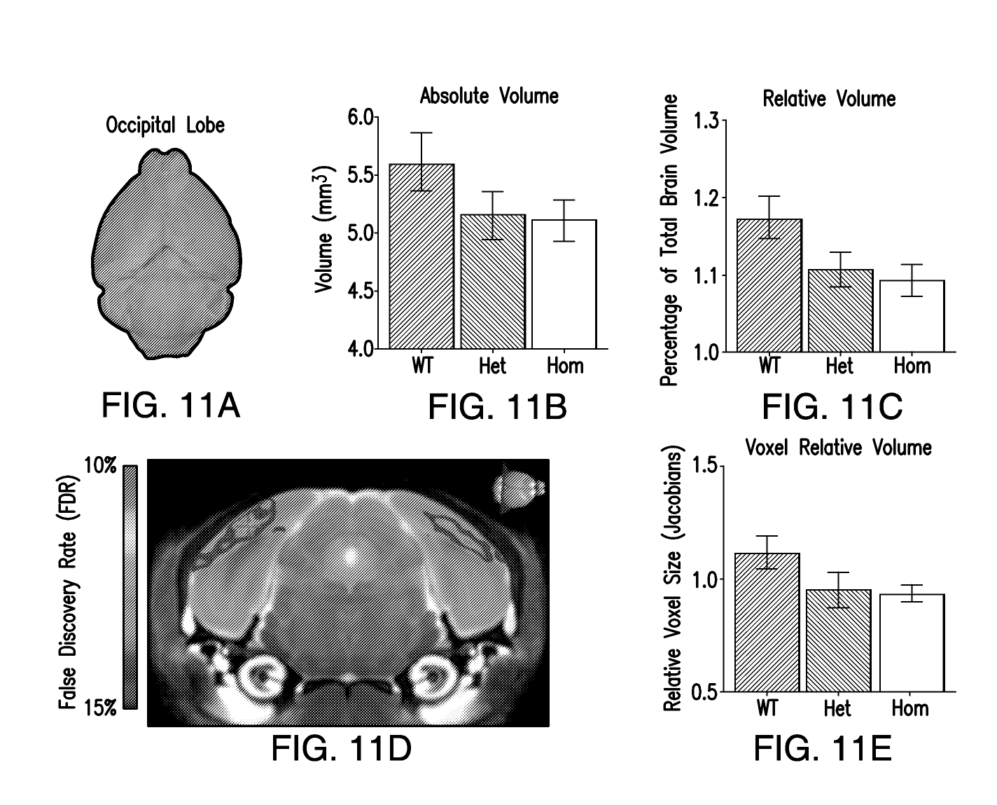

We were able to identify an isolated reduction in relative volume of the occipital lobe, which held up to multiple comparisons (FDR values of 0.08 for Caspr2^{+/-} mice and 0.01 for the Caspr2^{-/-} mice; FIG. 11 A-C). In order to characterize this finding further, we then masked out the voxel-wise changes and limited the comparisons to within the occipital lobe. This allowed us to determine where in the occipital lobe the volumetric reductions are localized (FIG. 11 D, E). Interestingly, a recent study reported a reduction in occipital cortex volume in subjects who were homozygous for the CNTNAP2 rs7794745 risk allele17, implying that the occipital lobe may be commonly affected in patients with CNTNAP2 CNVs or mutations.

### 7 EXAMPLE

### Rescue by mTOR Kinase Inhibitor

Rescue Experiments Utilizing WYE125132, an mTOR Kinase Inhibitor, to Target Disease Mechanism of Associated Neuropsychiatric Disorders in CNTNAP2(Caspr2) Mouse Model.

Since we identified the overactivation of the mTOR pathway as the likely disease mechanism in CNTNAP2-associated schizophrenia and autism-spectrum disorders in human subjects, we next carried out a series of experiments utlizing WYE125132, a specific mTOR kinase inhibitor compound, to rescue several of the critical disease-associated phenotypes in the Caspr2 mutant mice. The implication of this research is that WYE125132 has the potential to prevent and or reverse the molecular-, synaptic-, cellular-, and neurocircuitry level abnormalities associated with a wide variety of neuropsychiatric disorders associated with CNTNAP2 and other gene mutations which lead to overactivation of the mTOR pathway in human subjects. This group of neuropsychiatric disorders potentially includes, but is not limited to schizophrenia, autism-spectrum disorders, mood disorders, attention-deficit hyperactivity disorder, OCD and Tourette's, cognitive dysfunction or mental retardation, and seizure disorder.

Rescue of Enlarged Soma Size in Caspr2 Mutant Mice with WYE125132.

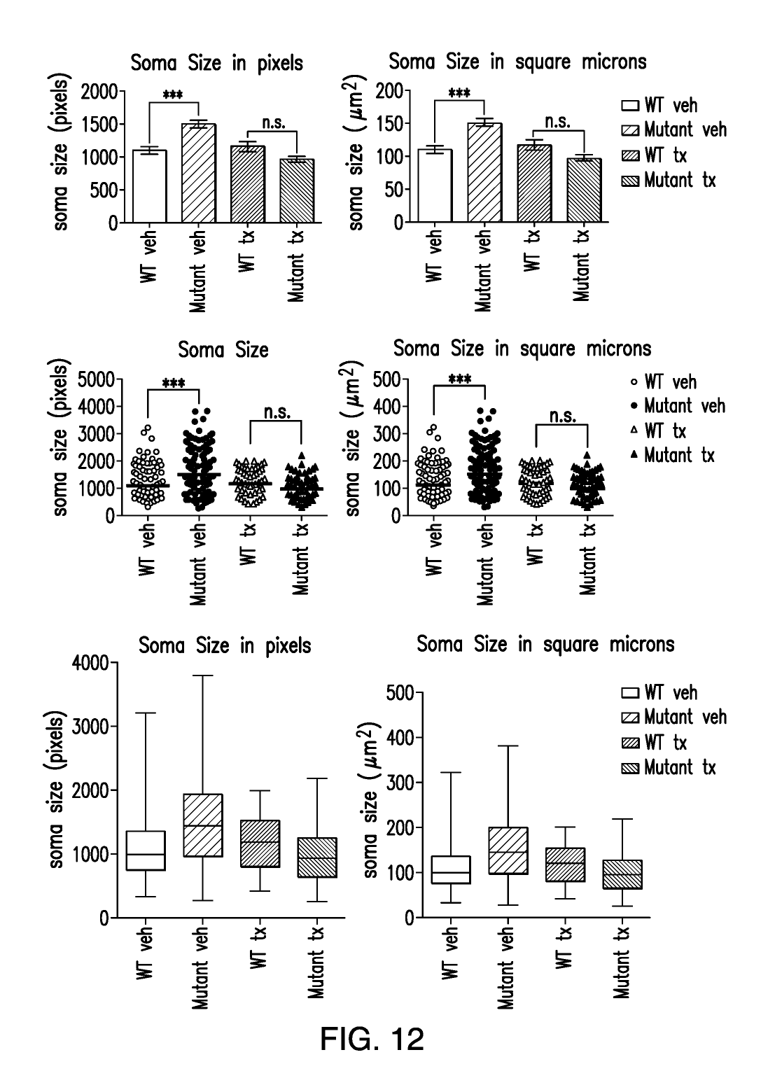

In order to assess whether we could reverse or prevent the cellular abnormalities (i.e., enlarged pyramidal cell size), we treated with WYE125132 (‘Tx’ in FIG. 12) or vehicle (‘veh’ in FIG. 12) Caspr2^{+/-}/Thy1-YFP/H and Caspr2^{-/-}/Thy-1-YFP/H mice and WT littermates also positive for YFP/H for the entire period P12-P35. The mice were perfused and subsequently fixed with 4% PFA and sectioned coronally at 16 microns. Subsequently, 200× images were taken using an epifluorescent microscope of somatosensory and auditory cortex—a total of 15 images per animal/cortical region. The cell somata were traced on Photoshop to quantify pixels. We then converted the pixels to square microns. For the analysis listed here, Caspr2^{+/-}/Thy1-YFP/H and

Caspr2^{-/-}/Thy-1-YFP/H mice were grouped together as ‘mutant’ mice (see FIG. 12). In FIG. 12 we present a summary of the data for vehicle- and WYE125132-treated animals and soma size. We ran ANOVA on the data sets as well as paired t-tests. One-way ANOVA clearly indicated that there is a significant difference across all datasets (P<0.0001). There was no significant difference in pyramidal cell size between WT and mutant compound-treated, indicating that treating with WYE125132 for approximately 3 weeks completely reverses the enlarged pyramidal cell size in Caspr2 mutants. There is a statistically significant difference (P<0.0001) between WT and Caspr2 mutants treated with vehicle, as expected from the data from the untreated.

Rescue of Abnormalities in Spine Dynamics in Caspr2 Mutant Mice with WYE125132.

In order to assess whether we could reverse or prevent the abnormalities in spine dynamics in Caspr2 mutants, we treated Caspr2^{+/-}/Thy1-YFP/H, Caspr2^{-/-}/Thy-1-YFP/H mice, and WT littermates also positive for YFP/H from P12-P32 with either WYE125132 or with vehicle. One-month old (P30±1) male mice were used in the experiments. Spine formation and elimination were examined by imaging the mouse cortex through a thinned-skull window as described previously. Briefly, 1-mo old male mice expressing YFP were anaesthetized with ketamine and xylazine (intraperitoneal; 20 mg/ml and 3 mg/ml, respectively, in saline; 6 μl per gram of body weight). Thinned-skull windows were made in head-fixed mice with high-speed microdrills. Skull thickness was reduced to about 20 μm. A twophoton microscope tuned to 920 nm (×60 water immersion lens; numerical aperture,1.1) was used to acquire images. For re-imaging of the same region, thinned regions were identified on the basis of the maps of the brain vasculature. Microsurgical blades were used to re-thin the region of interest until a clear image could be obtained. The area of the imaging region was 200 μm×200 μm in the frontal association cortex. The centers of the imaging regions were as follows: +2.8 mm bregma, +1.0 mm midline.

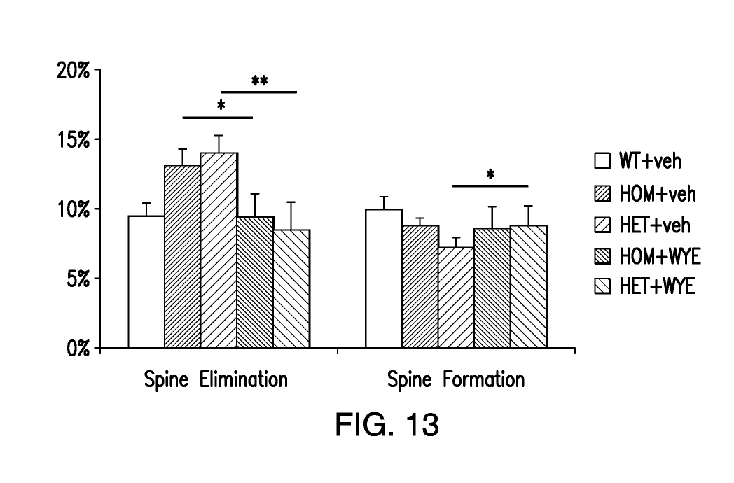

The results are shown in FIG. 13. Treatment with WYE125132 led to a dramatic prevention of spine elimination in Caspr2^{+/-}/Thy1-YFP/H (Caspr2^{+/-}/Thy1-YFP/H treated with vehicle are indicated in green while Caspr2^{+/-}/Thy1-YFP/H treated with WYE125132 are indicated in purple; P<0.006) and also a statistically significant prevention in Caspr2^{-/-}/Thy-1-YFP/H mice (Caspr2^{-/-}/Thy-1-YFP/H treated with vehicle are indicated in red while Caspr2^{-/-}/Thy-1-YFP/H treated with WYE125132 are indicated in turquoise; P<0.04). Treatment with WYE125132 also led to an increase in spine formation in the Caspr2^{+/-}/Thy1-YFP/H mice (P<0.0058). Together, these results indicate that treatment with WYE125132 for less than 3 weeks was able to prevent the abnormalities in spine dynamics almost completely in the Caspr2^{+/-}/Thy1-YFP/H mice and, to a lesser extent, also in the Caspr2^{-/-}/Thy-1-YFP/H mice.

Rescue of the Electrophysiological Abnormalities in the Caspr2 Mice Using WYE125132: Excitation/Inhibition Imbalance and LTP.

In addition to the previous electrophysiological experiments that we conducted in Caspr2 mutant mice in the age range of P21-P28, which demonstrated a shift away from cortical inhibition towards more excitation (see earlier descriptions), we also carried out complementary electrophysiological studies in 4-6-mo old mice. All mice were treated with either WYE125132 or vehicle for a period of 2 weeks after which the animals were euthanized and thalamocortical slices were prepared. Briefly, animals were anesthetized, decapitated and the brain quickly placed into ice-cold dissection buffer containing (in mM): 75 sucrose, 87 NaCl, 2.5 KCl, 1.25 NaH₂PO₄, 0.5 CaCl₂, 7 MgCl₂ 6H₂O, 25 NaHCO₃, 10 dextrose, bubbled with 95% O₂/5% CO₂ (pH 7.4). Slices (400 μm) were prepared with a vibratome (Leica, VT1200S), placed in 33-35° C. for artificial cerebrospinal fluid (ACSF, in mM: 124 NaCl, 2.5 KCl, 1.25 NaH₂PO₄, 2.5 CaCl₂, 1.5 MgSO₄ 7H₂O, 26 NaHCO₃, and 10 dextrose) for <30 min; then returned to room temperature >1 hr before use. Slices were then transferred to the recording chamber and perfused (2.0-2.5 ml min-1) with oxygenated ACSF at 33-35° C. and given 30 min to stabilize. Somatic whole-cell recordings were made from layer 5 pyramidal cells in voltage and current clamp mode with a Multiclamp 700B amplifier (Molecular Devices). Selection was based on morphology and electrophysiological criteria. Patch pipettes (3-8MΩ) contained a current clamp solution. Data were filtered at 2 kHz, digitized at 10 kHz and analyzed with Clampfit. Cells were excluded from analysis if Ri or Rs changed by >25% over the course of the recording. Excitatory post-synaptic currents or potentials (EPSCs/Ps) were evoked by extracellular stimulation (0.01-1 ms, 1-10V) with a 4×1 array of electrodes placed in layer 4, and straddling the patched layer 5 neuron. Across mice we compared spontaneous and evoked inhibitory responses by clamping the membrane potential of the cell to sub-threshold levels. Furthermore, we compared inhibition generated by each stimulation electrode, to the corresponding amount of excitation when the cell was held at −80 mV. Lastly, we tested Spike-Timing Dependent Plasticity (STDP).

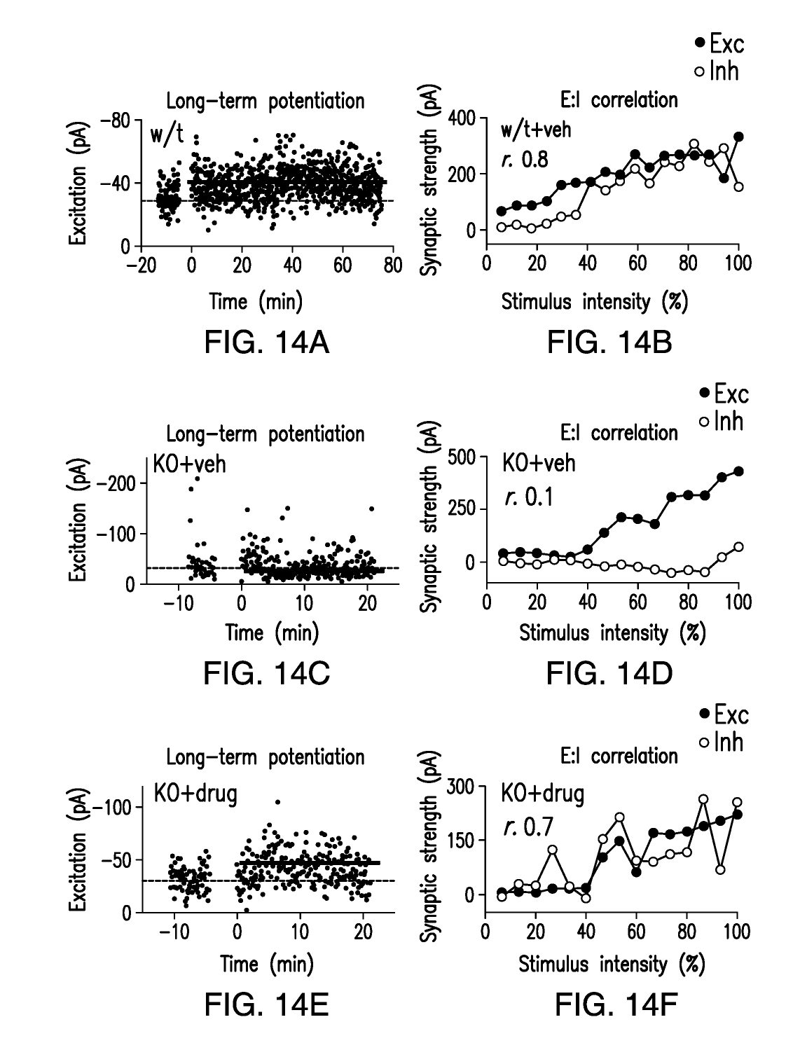

The results of this experiment are delineated in FIG. 14, where representative recordings from Caspr2 mutants and WT littermates are shown. LTP was induced with repetitive pre-post spike pairing as in STDP (FIG. 14A, C, E). Both WT littermates treated with vehicle (‘w/f’; FIG. 14A) or WYE125132, as well as Caspr2^{-/-}/Thy-1-YFP/H mice treated with WYE125132 (‘KO+drug’ in FIG. 14 E) demonstrated robust LTP, whereas Caspr2/Thy-1-YFP/H mice treated with vehicle (‘KO+veh’; FIG. 14C) did not. This indicates that Caspr2^{-/-}/Thy-1-YFP/H mice have impaired LTP, and that treatment with WYE125132, even if only for two weeks, is able to rescue this. Since we can pharmacologically target this abnormality in synaptic plasticity which likely underlies the cognitive deficits previously described in Caspr2 mutants (Penigarikano et al., 2011), we could potentially also rescue the cognitive impairments in the Caspr2 mutant mice.

In addition to the LTP assessment in this cohort of mice, when assessing the excitation/inhibition balance in slice by measuring both excitation and inhibition in voltage clamp by turning up stimulation, we observed a high correlation between excitation and inhibition in both WT littermates treated with vehicle (‘w/t+veh’; FIG. 14B) or WYE125132 as well as Caspr2^{-/-}/Thy-1-YFP/H mice treated with WYE125132 (‘KO+drug’ in FIG. 14F). However, Caspr2^{-/-}/Thy-1-YFP/H mice treated with vehicle (‘KO+veh’; FIG. 14D) did not show this correlation between excitation and inhibition, indicating that the imbalance between excitation and inhibition that we observed in mice at P21-28 is indeed also present in these 4-6-mo old mice, and that this excitation/inhibition imbalance can be reversed by treating mice for 2 weeks with WYE125132.

### 8. EXAMPLE

### Overactivation of the mTOR Pathway Mrediates the Autism-Related Pathophysiology in the CNTNAP2 MODEL

8.1 Materials and Methods

Mice.

Cntnap2 mutant and WT mice were obtained from heterozygous crossings and were born with the expected Mendelian frequencies. The three obtained genotypes were housed together. Mice were treated with p.o. gavaging once per day with either WYE125132 (10 mg/kg) or vehicle. All procedures involving animals were performed in accordance with the Columbia University/New York State Psychiatric Institute animal research committee.

Drug Administration.

WYE125132 (Chemscene) was administered by a daily p.o. gavaging in a volume of 10 ml/kg. For all behavioral experiments, dams were treated from PO to P12 after which individual pups were gavaged on a daily basis going forward. Mice also received drug treatment on the days of testing, at least 1 hour prior the experiment. For the two-photon and cell size quantitation studies, mice were gavaged from P12 to p35, after they were

euthanized. For the electrophysiology and western blot rescue experiments, adult mice were gavaged for 14 consecutive days after which they were euthanized.

Behavioral Tests.

Mice were marked with ear tagging. Experimenters were blinded to the genotype during testing. Behavioral tests were performed in the New York State Psychiatric Institute behavioral test core facility.

Pilocarpine-Induced Seizure Threshold Analysis.

Animals were injected with a weightadjusted dose of intra-periotoneal (i.p.) pilocarpine hydrochloride (Sigma-Aldrich, St. Louis, Mo.), 300 mg/kg. In order to limit peripheral side effects, 30 minutes before pilocarpine injection, all mice were given atropine methyl nitrate (1 mg/kg, i.p., TCI America), a competitive muscarinic acetylcholine receptor antagonist that does not cross the blood-brain barrier.

Western Blot Analysis and Immunocytochemistry.

Western blot analysis of mouse brain tissue and immunocytochemistry in human brain tissue was performed using standard methods.

Electron Microscopy.

Human Brain Tissue.

All human brain tissue processing and preparation was carried out at the Mayo Clinic Electron Microscopy Core (1426 Guggenheim, X4-3148). Tissue was deparaffinized and subsequently prepared for electorn microscopy. Thin (90 nm) sections were cut on a Leica UC7 ultramicrotome, placed on 200 mesh copper grids and stained with lead citrate. Micrographs were taken on a FEI Tecnai™ transmission electron microscope 12 operating at 80 KV.

Mouse Brain Tissue.

We collected brains of 4 Cntnap2^{+/-} and 4 WT mice for electron microscopic analysis, ranging in age from 1.5 to 2 months. All members of the team of researchers engaged in the electron microscopic analysis were kept blind of genotype of the animals. Morphology was analyzed from digital images captured at a magnification of 60,000×, using the JEOL XL electron microscope, Hamamatsu's CCD camera and AMT's software. All data from the electron microscopic immunocytochemical quantification were analyzed using the software Statistica (version 10.0, released from StatSoft).

Soma Size Quantitation. Cntnap2 mice were crossed to Thy1-YFP in order to visualize layer V pyramidal neurons in the neocortex. Cntnap2+/−, Cntnap2−/− and WT mice were cardially

perfused with PBS and then 4% PFA and subsequently post-fixed in 4% PFA for 1 hour at 4° C. After cryoprotection with 30% sucrose in PBS, brains were embedded in TissueTek OCT (Takara) and were sectioned coronally on a cryostat at 16 μm thickness. Sections containing auditory and somatosensory cortices were analyzed. Sections were re-hydrated in PBS, blocked with 10% normal serum and permeablized in 0.1% Triton-X-100. GFP antibody (1:1500, Naclai-Tesque) was incubated in 0.1% Triton X-100, 4% normal serum in PBS overnight at 4° C. and visualized with donkey anti-rat FITC secondary antibody (1:1000, Jackson Immunoresearch). Fields of view in somatosensory and auditory cortices were
chosen at random in the DAPI channel and images were taken in the FITC channel. Soma size was determined by manually tracing pyramidal cell somata and measuring pixel number per selection. Pixel number was then converted to μm². For each animal, 90 to 140 somata were measured.

Electrophysiology.

Untreated Electrophysiology Experiments in Juvenile Mice.

Whole-cell recordings were made from randomly selected pyramidal cells located in upper layers (I-III) of the somatosensory cortex from animals aged P21-P31. Experiments were performed in currentclamp mode using the Axoclamp 2B or the Axopatch 200B amplifier (Molecular Devices) and in voltage clamp using the latter. Spontaneous synaptic currents were filtered at 3 kHz and recorded with a sampling rate of 10 kHz. Individually acquired sEPSCs and sIPSCs were

isolated by adjusting the voltage that the cell was held at, at −65 mV for sEPSCs and at 0 mV for sIPSCs. Passive and active membrane properties were recorded in current clamp mode by applying a series of sub- and supra-threshold current steps.

WYE125132 Treatment Experiments in Adult Mice.

Whole cell recordings were made from layer V pyramidal cells of the temporal cortex. Focal extracellular stimulation was applied and excitatory and inhibitory currents were measured across various stimulation intensities to assess E:I balance. For plasticity experiments, extracellular stimulation was paired with postsynaptic action potentials (elicited by current injection via the recording electrode) less than 10 ms after the onset of the EPSP 60 times at 0.1 hz. Baseline strength was compared to synaptic strength 6-15 mins after spike pairing.

MRI Imaging.

Mice were anesthetized with ketamine/xylazine and intracardially perfused with 30 mL of 0.1M PBS containing 10 U/mL heparin (Sigma) and 2 mM ProHance (a Gadolinium contrast agent) followed by 30 mL of ice cold 4% paraformaldehyde (PFA) containing 2 mM ProHance (Spring et al., 2007). After perfusion, mice were decapitated and the skin, lower jaw, ears, and the cartilaginous nose tip were removed. The brain and remaining skull structures were incubated in 4% PFA+2 mM ProHance overnight at 4° C. then transferred to 0.1M PBS containing 2 mM ProHance and 0.02% sodium azide for at least 7 days prior to MM scanning (Cahill et al. Neuroimage 2012). A multi-channel 7.0 Tesla MM scanner (Varian Inc., Palo Alto, Calif.) was used to image the brains within skulls.

FDG-microPET/MRI Image Analysis.

All imaging was carried out by the staff at the Center for Molecular and Genomic Imaging (CMGI University of California, Davis) under an approved animal use protocol. The mice were anesthetized with a mixture of isoflurane and oxygen gas (˜1-1.5%) for a short period for injection of the FDG. Animals were re-anesthetized immediately prior to scanning and secured onto an imaging bed. Image analysis was performed as previously described (Thanos et al. 2008; Pascau et al. 2009).

In Vivo Transcranial Two-Photon Microscopy.

One-month-old (P30±1) male mice were used in the experiments. Spine formation and elimination were examined by imaging the mouse frontal association cortex through a thinned-skull window as described previously (Lai, 2012).

8.2 RESULTS

Cntnap2 Mutant Mice Show Altered Social Interaction:

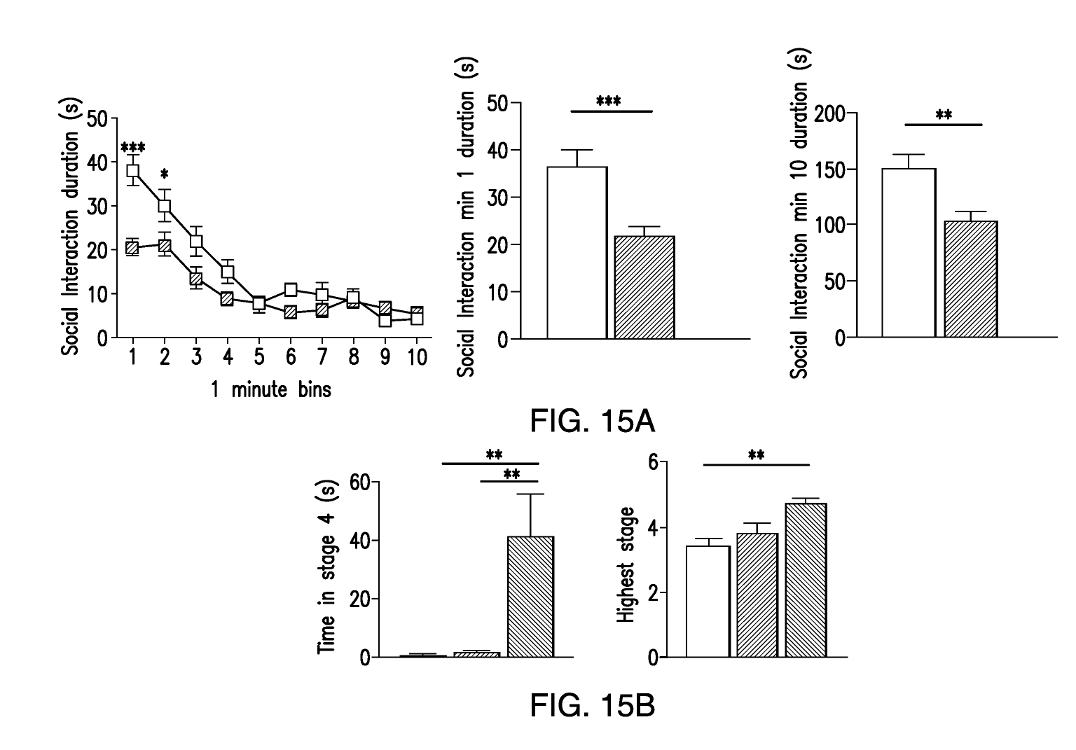

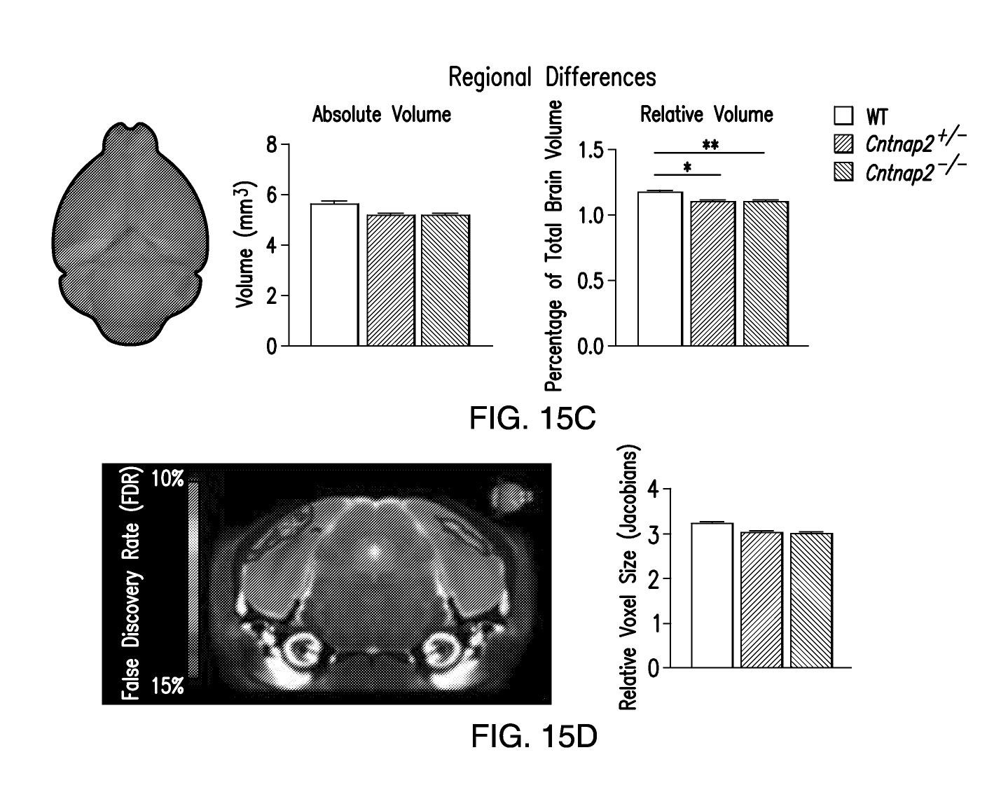

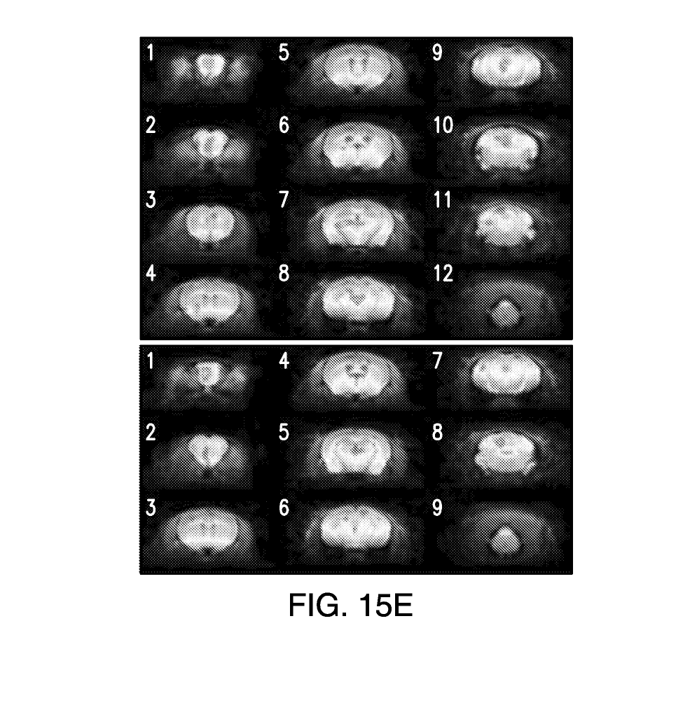

Detailed measurements of interaction between pairs of mice placed together in standard cages provide insights into reciprocal social interactions (Silverman et al., 2010). We examined (i) whether juvenile Cntnap2 mutant mice display decreased social interaction over a 10-minute interval and (ii) whether mutant mice have reduced tendency to approach novel social stimuli, as evidenced by social inhibition in the first minute of the tested interval (Curley et al., 2009). The first minute of this social interaction paradigm represents the phase when preference for social novelty is normally established. There were significant differences between genotypes in the amount of time spent on social investigation of the stimulus animal both in the first minute of the test (One-Way ANOVA F2,38=5.01, P<0.05; FIG. 15A) as well as over the entire 10-minute test (F2,38=3.27, P<0.05; FIG. 15A). Both Cntnap2^{+/-} and Cntnap2^{-/-} mice investigated the stimulus mouse for less time than their wild-type (WT) littermates (Dunnett post-hoc tests, all P<0.05).

Cntnap2 Mutant Mice have a Lower Threshold to Pilocarpine-Induced Seizures:

Individuals carrying CNTNAP2 mutations commonly have seizures (Strauss et al., 2006; Friedman et al., 2008) and CNV studies have identified genetic lesions of CNTNAP2 in individuals with epilepsy (Mefford et al., 2011). Although some 6-12 mo old Cntnap2 mutant 8 mice were observed to have spontaneous generalized tonic-clonic seizures, this was a rare occurrence and we were not able to elicit a significant number of seizures by handling or audiogenic stimulation as previously reported (Peñagarikano et al., 2011). We therefore utilized a pilocarpine seizure induction protocol to test whether Cntnap2 mutant mice display lower seizure threshold compared with WT littermates.

We found that Cntnap2 deficiency was strongly associated with both seizure severity and duration of seizures. Specifically, of all the animals tested (n=40), 92% (11/12) of Cntnap2^{-/-} mice and 47% (7/15) of Cntnap2^{+/-} mice reached stage 4 or higher on the seizure rating compared with only 23% (3/13) of WT littermates (see FIG. 15B). The effect of decreasing Cntnap2 gene dosage on the proportion of animals reaching stage 4 was statistically significant (Chi Sq linear-by-linear association, P=0.001). In addition, the Cntnap2^{-/-} genotype was associated with the most severe seizure stage reached on a 5-point scale (Chi Sq linear-by-linear association, P=0.001). The median highest stage reached (Independent samples median test, P=0.002) and the distribution of highest stage reached (Independent samples Kruskal-Willis Test, P=0.03) differed significantly among genotypes. When looking at stage 4 seizures, the mean duration was 0.46 minutes for WT mice compared with 1.80 minutes for Cntnap2^{+/-} and 41.25 minutes for Cntnap2^{-/-} mice. As with measures of severity above, the time to onset of stage 4 seizures (Independent samples Kruskal-Wallis test, P<0.009) and duration of stage 4 seizures (Kruskal-Wallis test, P<0.025) differed significantly across genotypes. In summary, decreased Cntnap2 dosage conferred increased susceptibility to pilocarpine-induced seizures, with effects on both severity and duration.

Overall Normal Neuroanatomy with a Selective Occipital Cortex Reduction in Cntnap2 Mutant Mice:

Cntnap2 is expressed in multiple adult brain regions, primarily cerebral cortex, hippocampus, striatum, olfactory tract, and cerebellar cortex (Peñagarikano et al., 2011). Utilizing a multi-channel 7.0 Tesla MRI scanner, we examined sixty-two different brain regions to determine changes in both absolute volume (mm³) as well as relative volume (% total brain volume). We found that mesoscopic neuroanatomy as well as fractional anisotropy and other diffusion measurements were grossly normal with no changes in absolute volume in any specific brain region in Cntnap2 mutant mice. In terms of relative volume, there was a specific volumetric deficit in the occipital cortex [False Discovery Rates (FDRs): 8% for Cntnap2+/− and 1% for Cntnap2−/− mice] (FIG. 15 C, D). Voxel-wise changes throughout the entire brain were also investigated and no significant differences were found. When the occipital lobe was examined independently for voxel-wise differences to decrease the multiple comparisons, a bilateral volumetric deficit was found, with FDR rates ranging from 10-15% (FIG. 15D).

Cortical and Subcortical Hypermetabolism in Cntnap2 Mutant Mice:

We compared changes in regional brain glucose metabolism in Cntnap2 mutant mice and WT littermates using micro-positron emission tomography (microPET) with [18F]2-fluoro-2-deoxy-D-glucose co-registered with structural Mill images. Both Cntnap2^{+/-} and Cntnap2^{-/-} mutant mice show a strikingly similar spatial pattern of hypermetabolism in specific brain regions, including large

areas of the cortex, thalamic nuclei, olfactory bulb, cerebellum, and hippocampus (FIG. 15E). When mutant mice were compared with each other, Cntnap2^{+/-} mice demonstrated a significantly larger degree of hypermetabolism in the prefrontal cortex, thalamic nuclei, and olfactory bulb (FIG. 15E). Furthermore, both Cntnap2^{+/-} and Cntnap2^{-/-} mutant mice show a more restricted pattern of hypometabolism confined in the piriform cortex, midbrain nuclei, and brain stem (FIG. 15E).

Abnormal Neuronal Network Activity and Plasticity in Cntnap2 Mutant Mice:

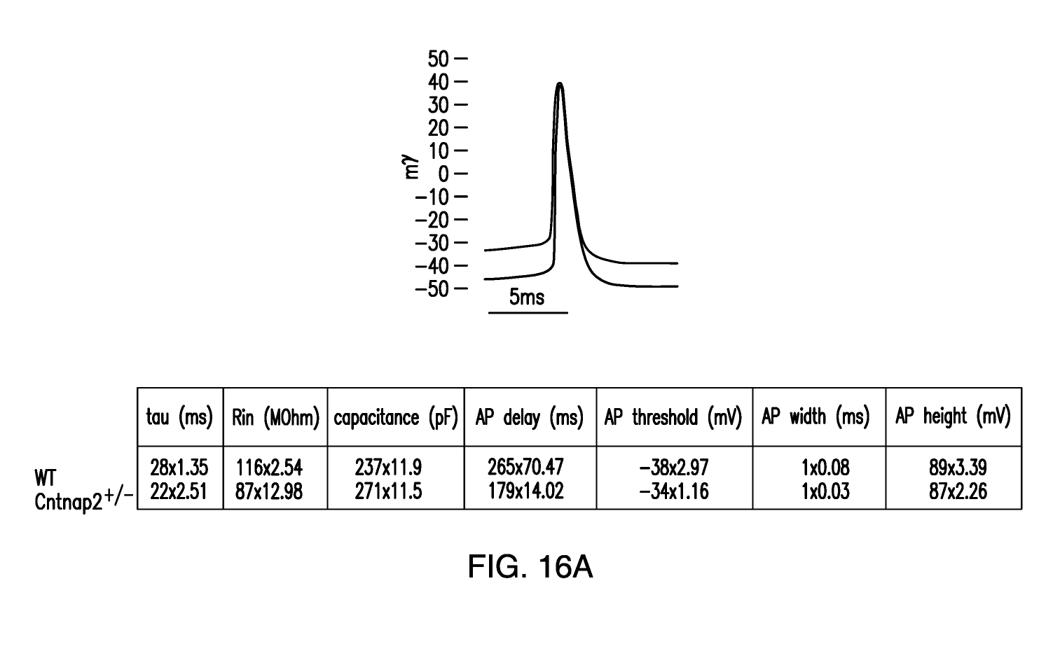

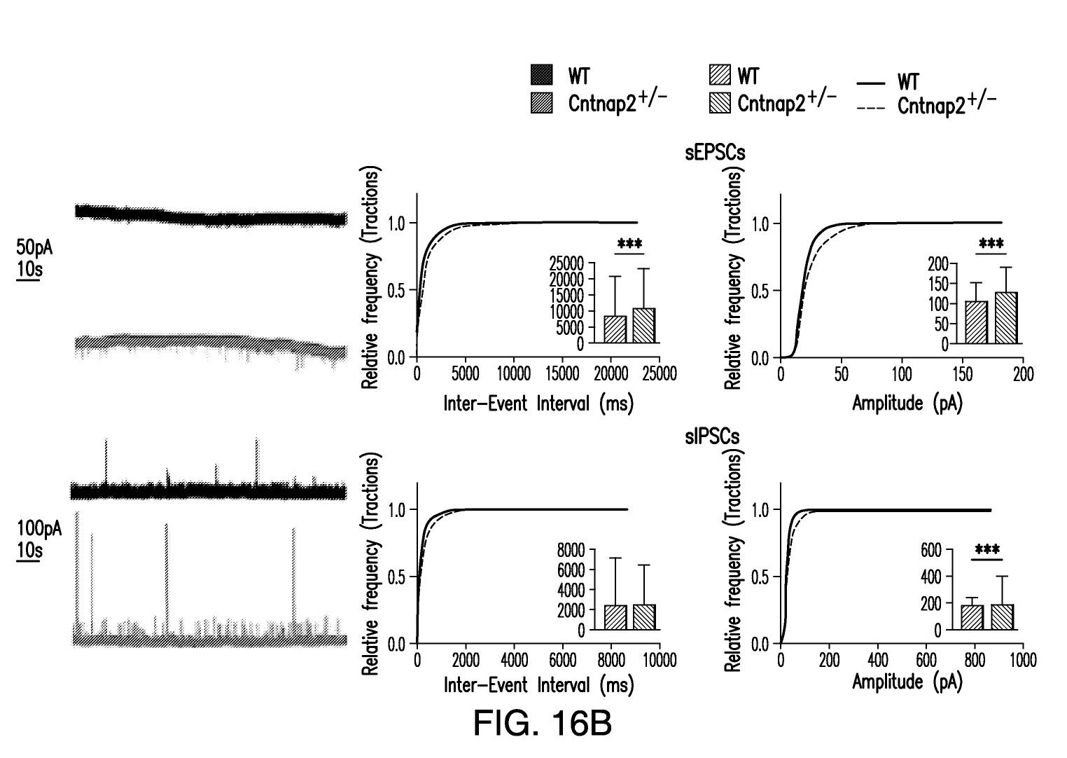

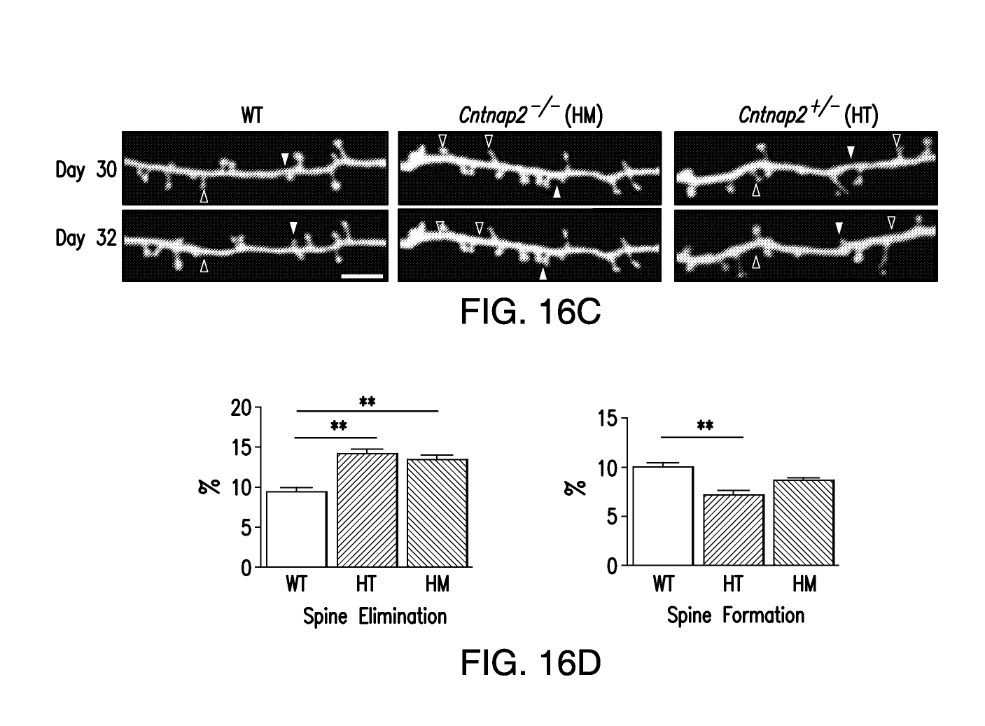

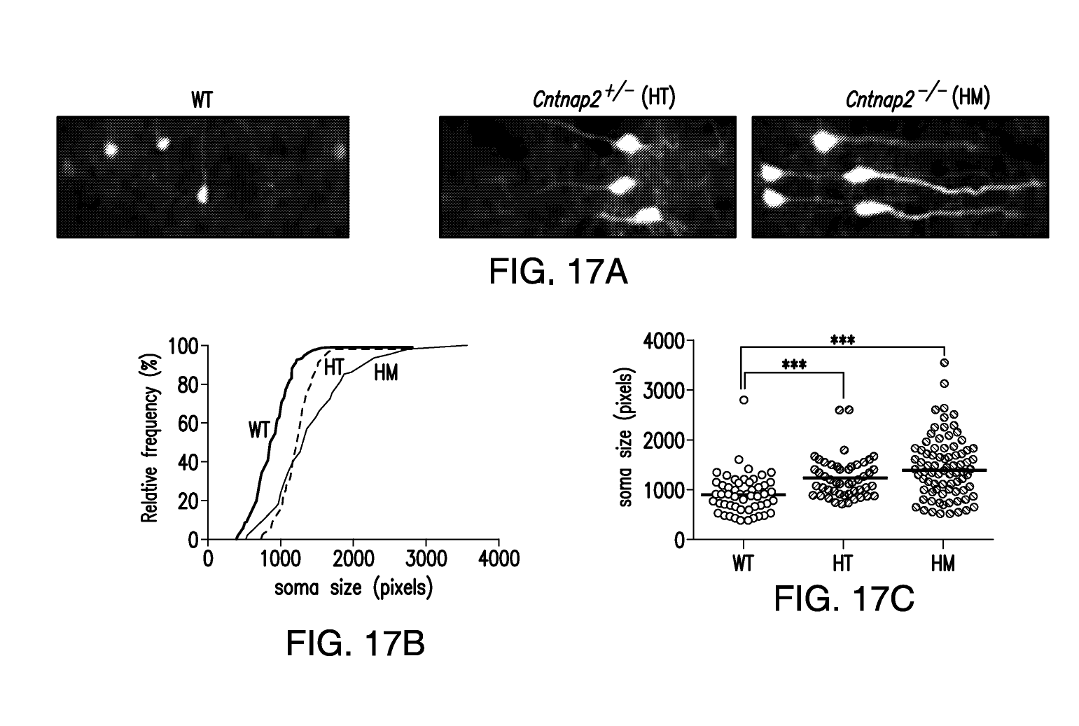

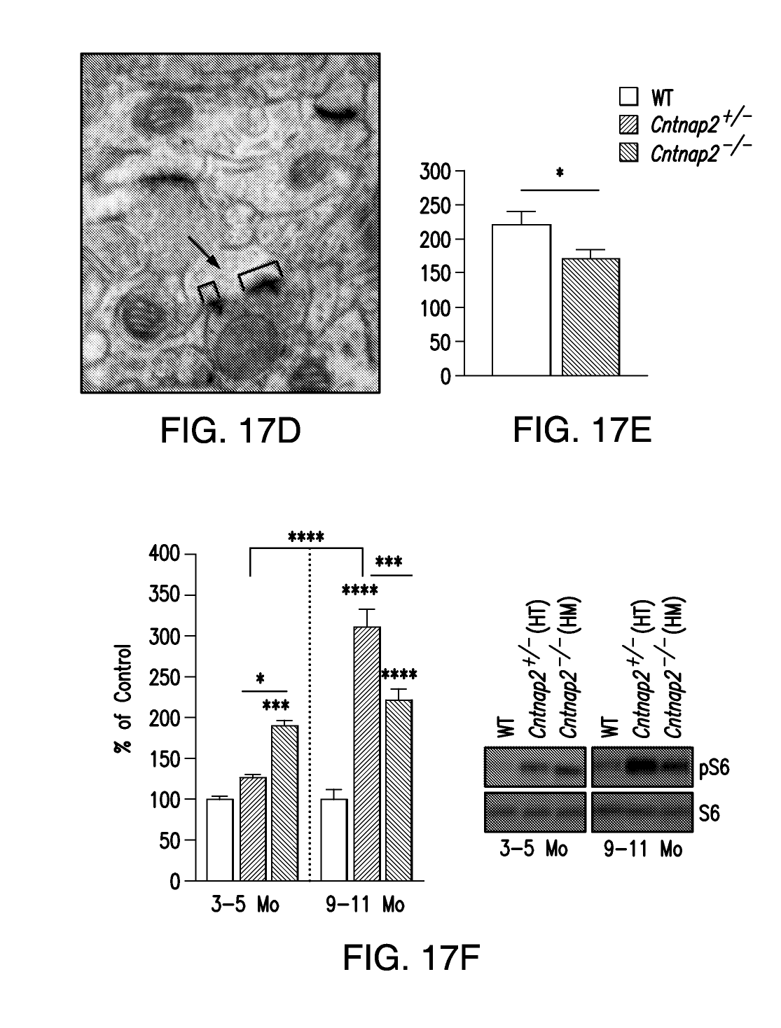

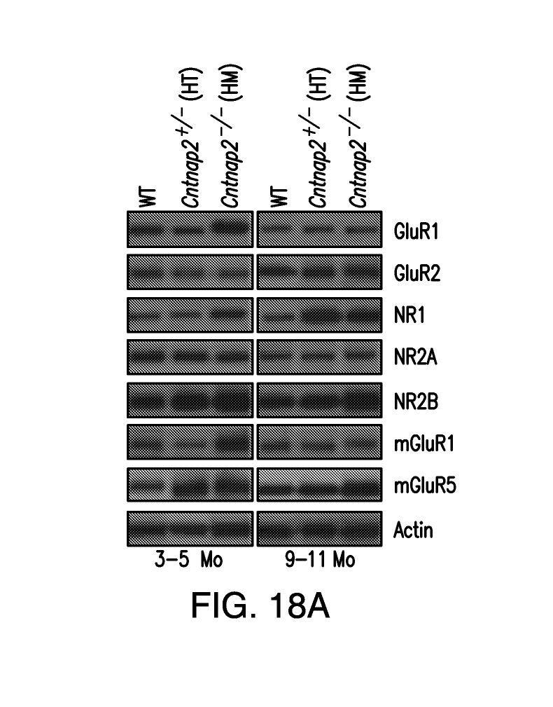

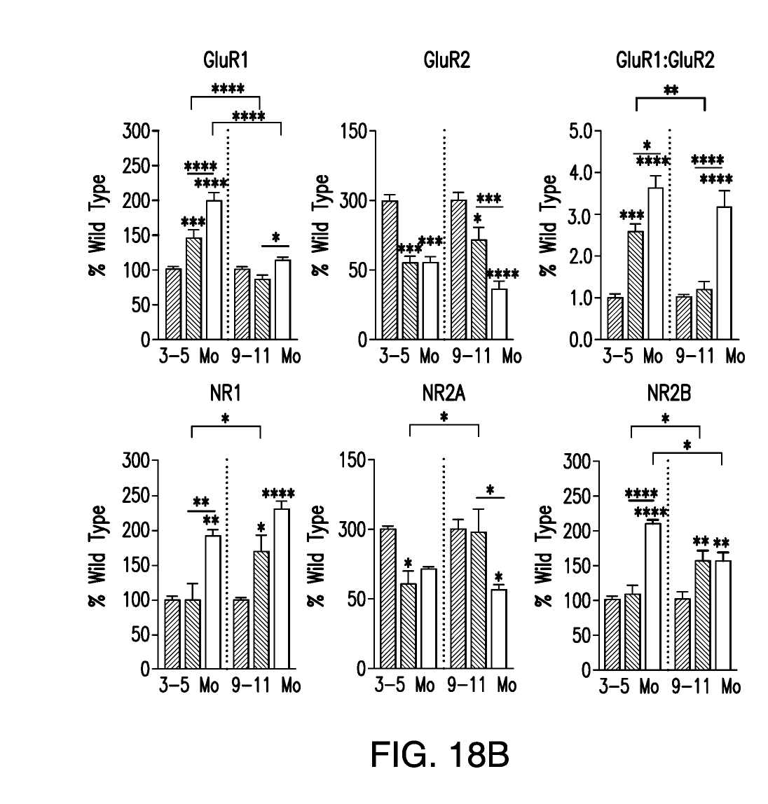

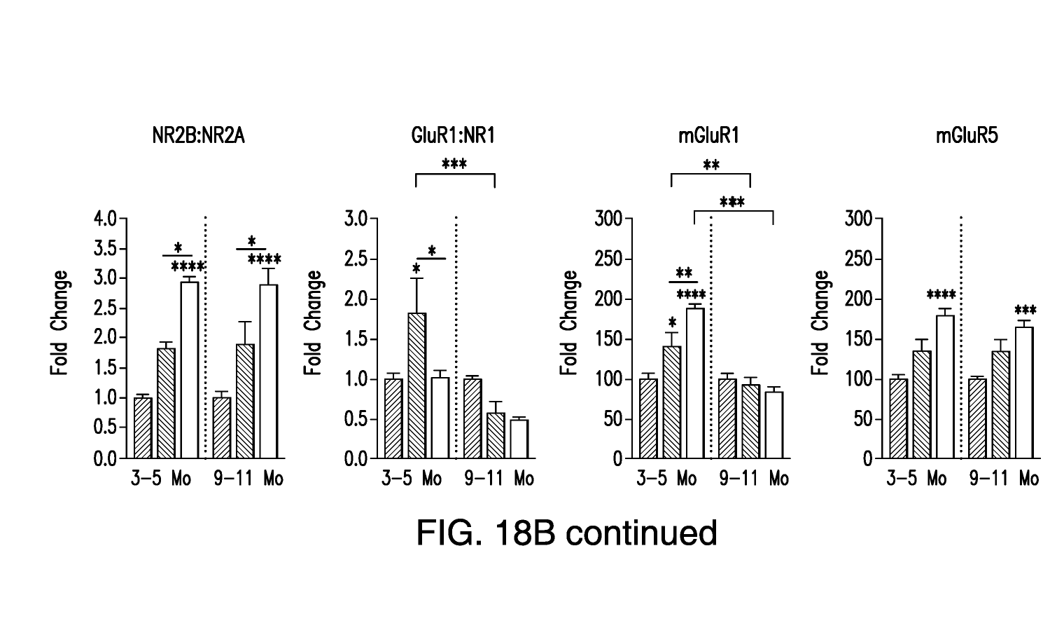

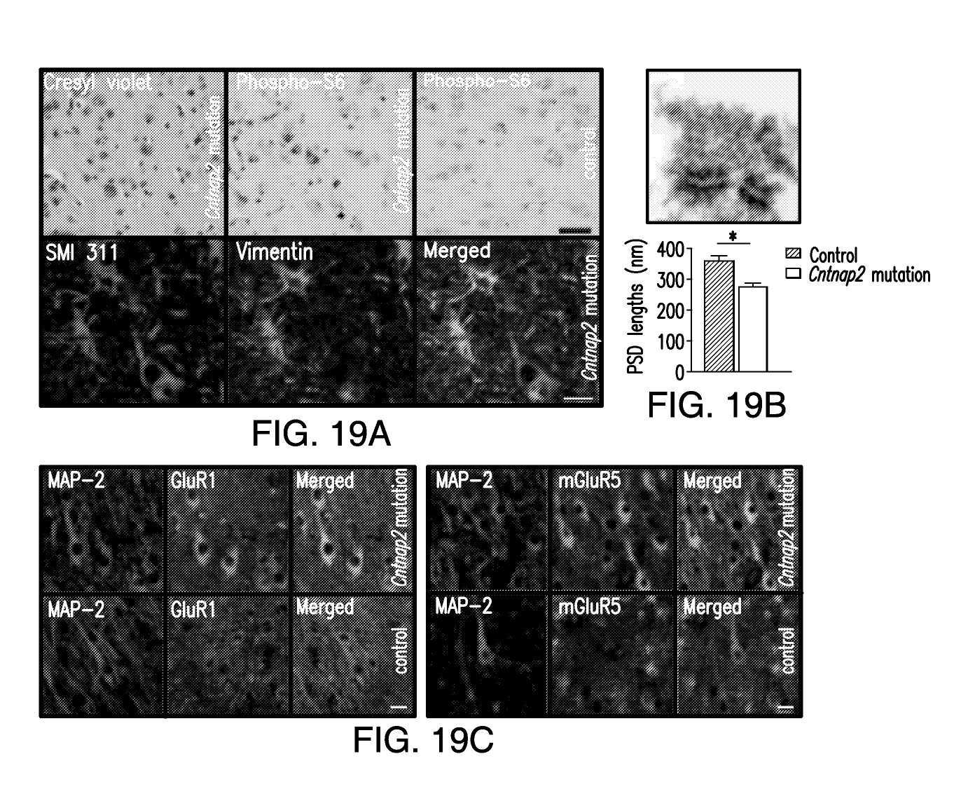

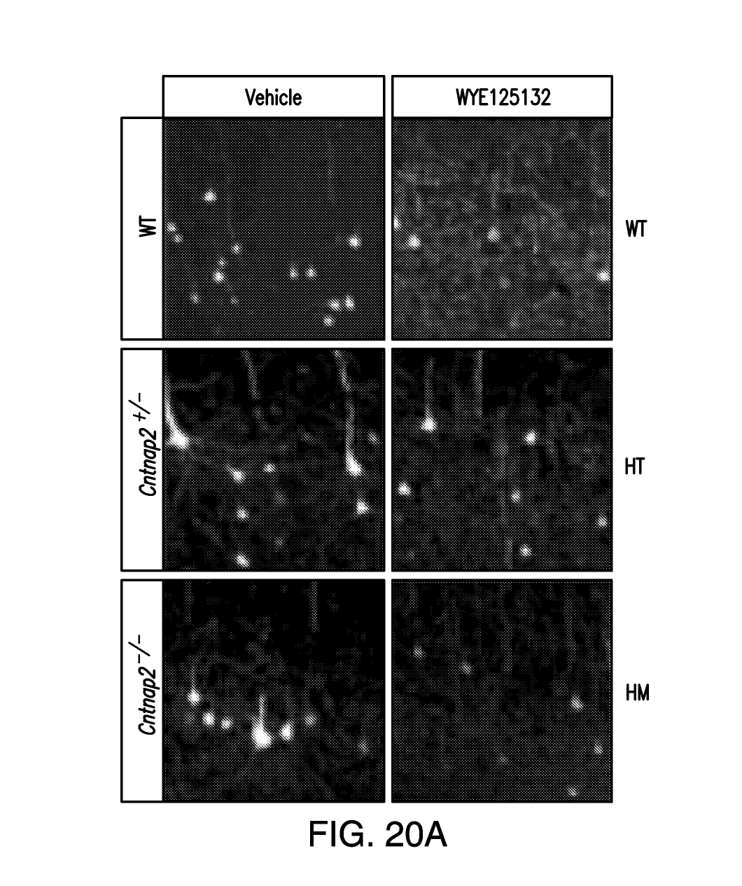

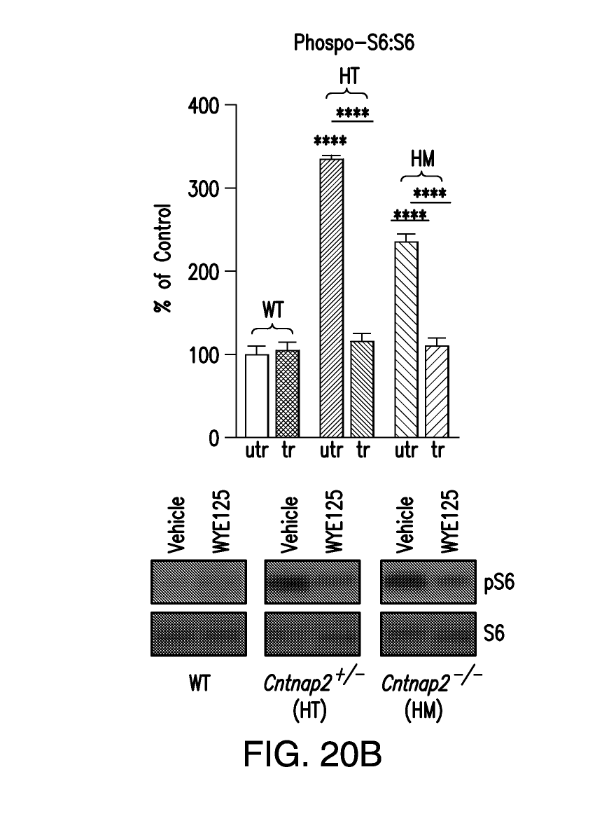

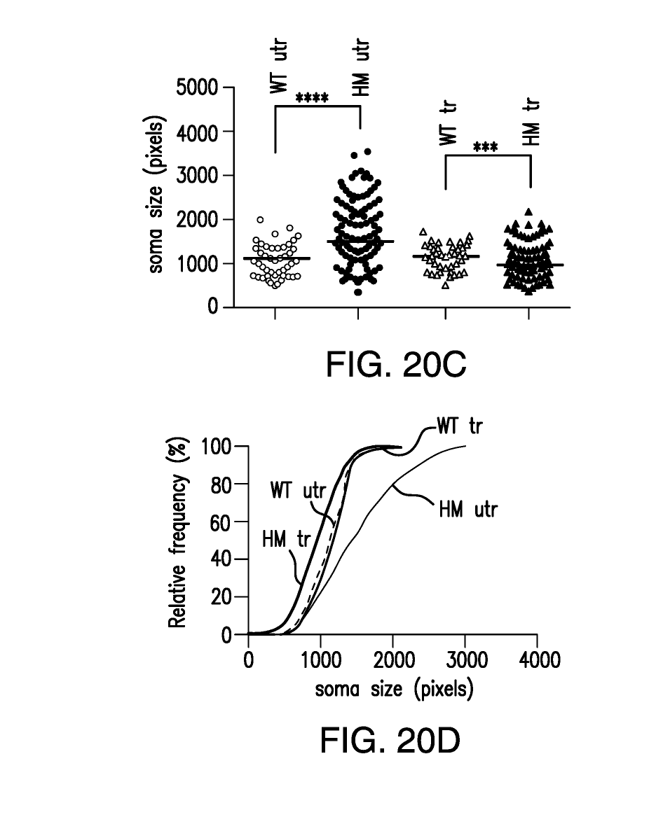

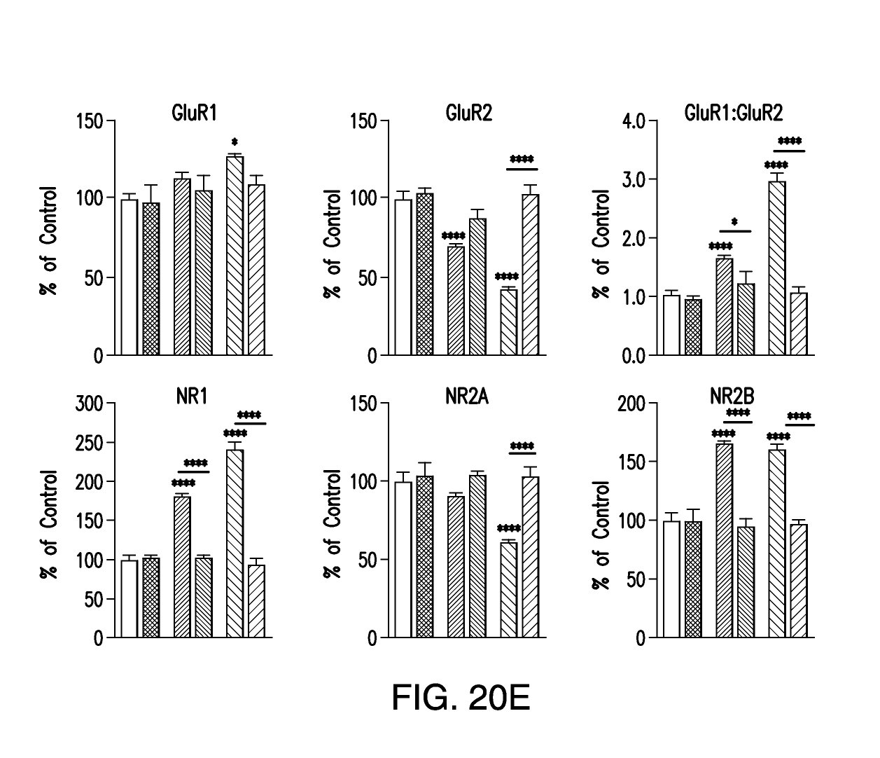

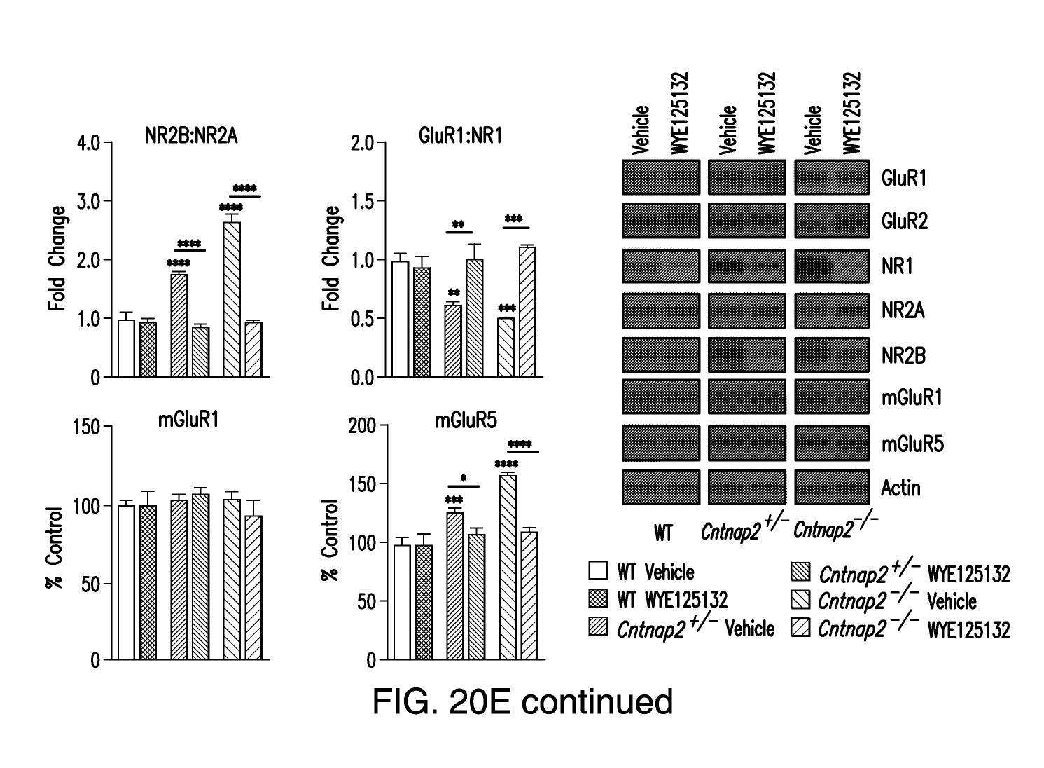

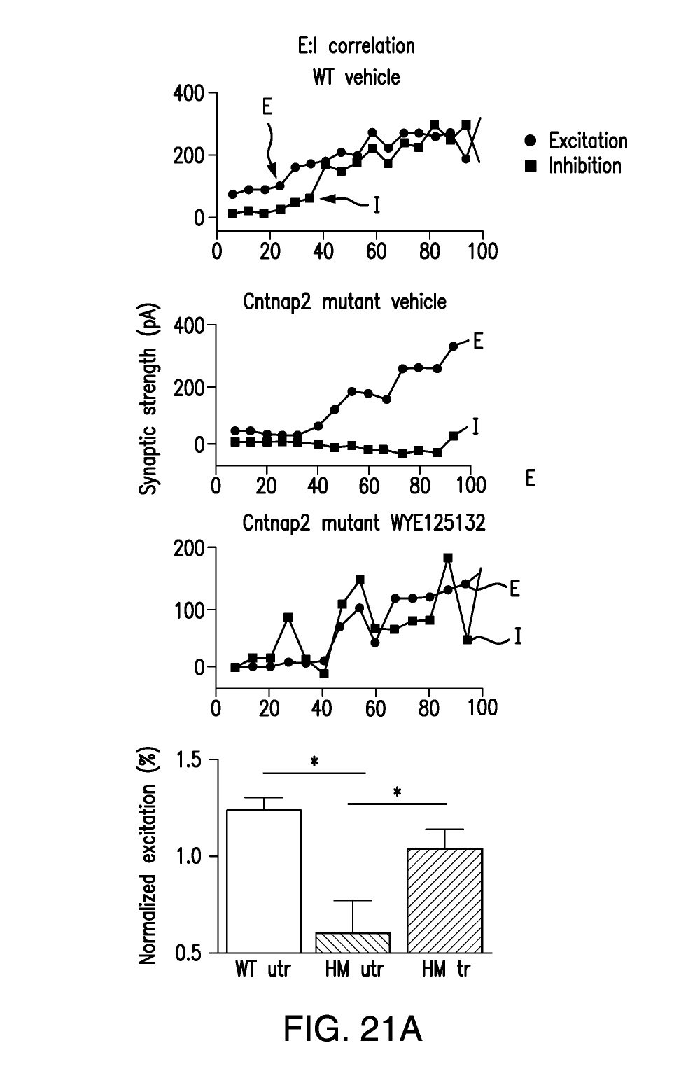

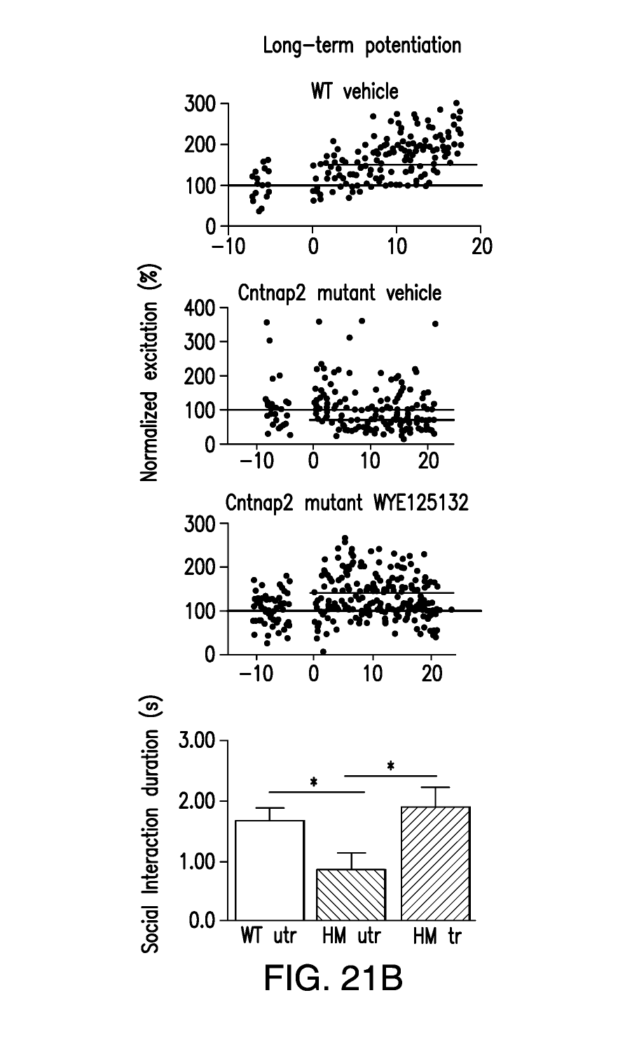

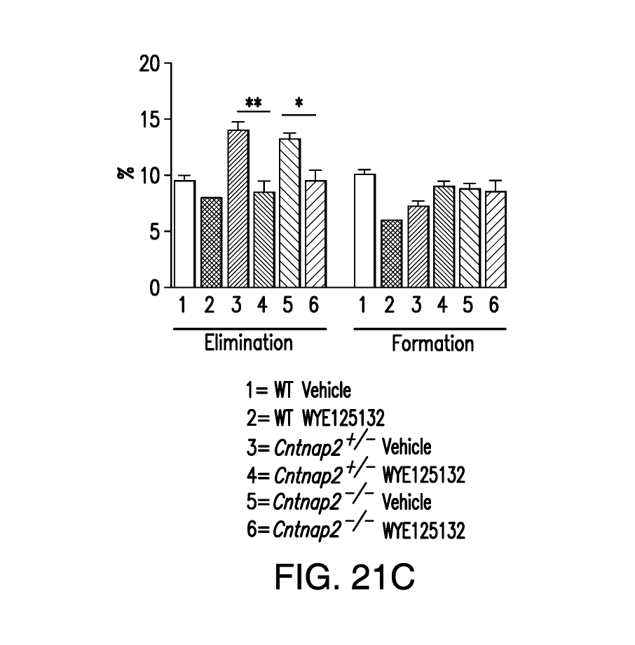

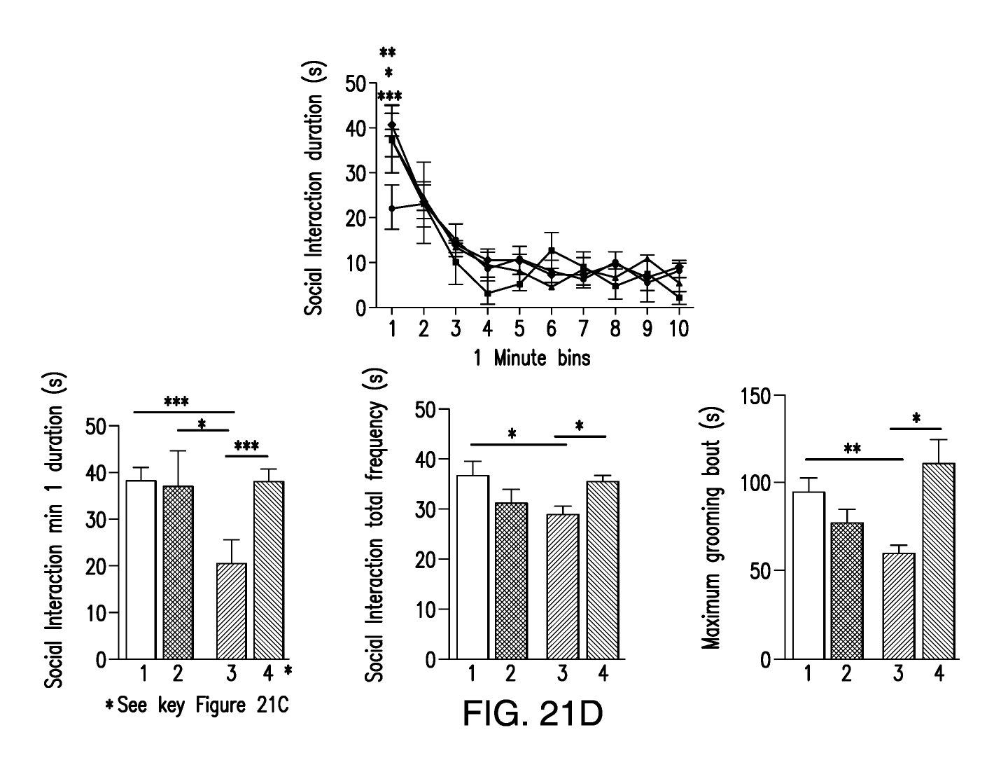

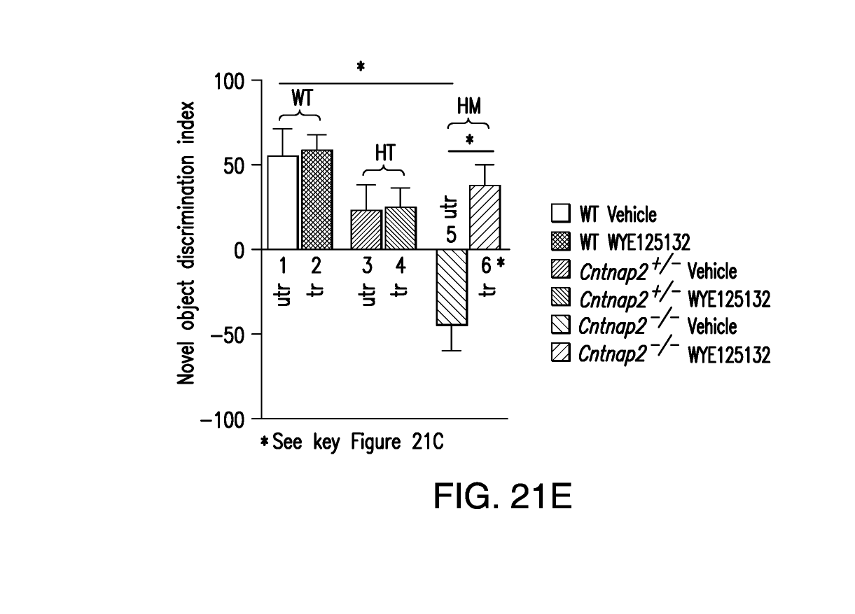

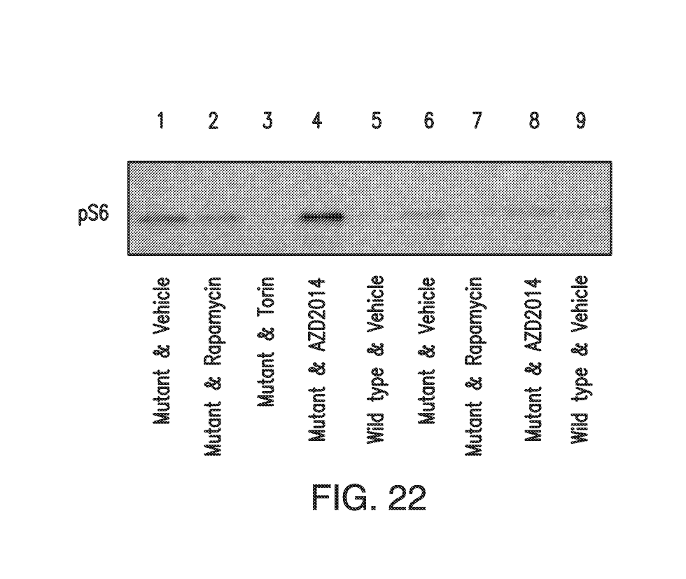

The observed behavioral deficits, lowered seizure threshold and regional pattern of metabolic abnormalities, together suggest that neural network activity and plasticity might be altered in Cntnap2 mutant mice. Therefore, we examined the excitation-inhibition balance in the cortex of juvenile (P21-31) mutant and WT mice. Neocortical excitation/inhibition imbalance may lead to defective information processing and social dysfunction (Yizhar et al., 2011) and may be an important neural correlate of behavioral deficits seen in patients with ASD (Rubenstein & Merzenich, 2003). We performed in vitro electrophysiology on pyramidal cells in the cortex of Cntnap2 mutant mice and WT littermates. As Cntnap2 has been shown to be important for the localization of potassium channels, we assessed the action potential characteristics and found that Cntnap2^{-/-} mice have a more depolarized voltage threshold compared WT littermates. This voltage difference is consistent with the importance shown for Cntnap2 in the clustering of voltage-gated channels in myelinated axons (Horresh et al., 2008). In contrast, other single action potential characteristics, such as the delay to first spike, the action potential half-width, the amplitude, and the after-hyperpolarization amplitude did not differ among genotypes (FIG. 16A). We hypothesized that the cortical hypermetabolism we observed with FDGmicroPET/MRI is due instead to a change in excitation and/or inhibition that projection neurons receive. To test this, we recorded and analyzed spontaneous excitatory and inhibitory postsynaptic currents (sEPSCs and sIPSCs) in vitro from layer V pyramidal cells of the frontal cortex.

Analysis of the amplitude and frequency distribution of sEPSCs in WT (n=3240 events in total) versus Cntnap2^{-/-} (n=2166 events in total) cells showed a decrease in frequency (P<0.0001) and an increase in amplitude (P<0.0001) in Cntnap2−/− mice (FIG. 16A,B). In contrast, when comparing the distribution of the amplitude and frequency of spontaneous inhibitory postsynaptic currents (sIPSCs) between WT (n=4061 events in total) and Cntnap2^{-/-}

(n=4385 events in total) cells, we found that there was a similar increase in the amplitude (P<0.0001), but with intact frequency (P=0.7624; FIG. 16B). Thus, Cntnap2 deficiency appears to lead to abnormalities in the excitation/inhibition balance in pyramidal cells.

The decrease in frequency of sEPSCs could be due to functional or structural deficits of synapses. In order to address this, we next examined Cntnap2 mutant mice for abnormalities in spine turnover. We utilized transcranial two-photon microscopy in the living, intact brain to check for abnormalities in the cortical spine dynamics, an index for glutamate-dependent stabilization of cortical neurocircuitry (Grutzendler et al., 2002). We examined the formation and elimination rates of dendritic spines on apical dendrites of layer V pyramidal neurons in the frontal association cortex. We repeatedly imaged 1-month-old Cntnap2^{+/-} and Cntnap2^{-/-} mutant mice as well as WT littermates within the Thy1-YFP genetic background over a 2-day interval to examine the turnover rate of dendritic spines. We found that the rates of spine elimination over 2 days were significantly higher in both Cntnap2^{+/-} and Cntnap2^{-/-} mutant mice compared with the age-matched WT littermates. The formation and elimination rates were 9.5±0.9% and 10.0±0.9% in WT littermates (n=4) compared with 7.2±0.7% and 14.1±1.2% in Cntnap2^{+/-} mice (n=3) and 8.8±0.5% and 13.2±1.1% in Cntnap2^{-/-} mice (n=4), respectively [One-Way ANOVA, Tukey, elimination: F (2,10)=18.620; formation: F(2,10)=12.682, P<0.05] (FIG. 16C,D). However, there were no significant differences in the turnover rate of filopodia

(P>0.05). The increase in elimination and decrease in formation of dendritic spines in Cntnap2 mutant mice is consistent with the decreased frequency of sEPSCs we find in these cells. Taken together, these results suggest that absence of one or both Cntnap2 alleles leads to abnormal structural synaptic plasticity during a developmental window when cortical neurocircuitry remodeling takes place.

Enlarged Pyramidal Cells Soma Size in Cntnap2 Mutant Mice:

We characterized cellular morphology in Cntnap2^{+/-}/Thy1-YFP/H and Cntnap2^{-/-}/Thy-1-YFP/H mice and compared it to WT/Thy1-YFP/H littermates. Examination of pyramidal cell morphology in layer V of auditory and somatosensory cortices in 2-mo old mice, revealed a pattern of dramatically enlarged pyramidal cell somata in all mutant animals examined (FIG. 17A-C) (Cntnap2^{+/-}/Thy1-YFP/H mice versus WT/Thy1-YFP/H littermates: P<0.0001; Cntnap2^{-/-}/Thy-1-YFP/H mice versus WT littermates: P<0.0001; Kruskal-Wallis one-way ANOVA).

Decreased Length of Perforated Postsynaptic Densities (PSDs) in Cntnap2 Mutant Mice:

Because many dendrites of pyramidal cells form synapses in layer I, we utilized electron microscopy to examine the synaptic structure in this layer of the frontal cortex of Cntnap2^{-/-} mice and WT littermates, ages P45-P60. We examined the length and width of both perforated and non-perforated PSDs, as well as the width of the synaptic cleft. Although we found no difference in the lengths or synaptic cleft distance of perforated or non-perforated synapses, we found a 22% decrease in the length of segmented PSDs of perforated synapses of Cntnap2^{-/-} mice compared to those WT control animals (Median Test or Kruskal-Wallis ANOVA by Ranks, p=0.002; FIG. 17D-E). Perforated synapses are large synapses that have been implicated in memory- and learning-related plasticity (Lamprecht & LeDoux, 2004). It is therefore likely that the decrease in length of the perforated PSDs is a structural synaptic correlate of the cognitive deficits seen in Cntnap2 mutant mice (Penagarikano et al., 2011) as well as the cognitive dysfunction observed in human subjects with CNTNAP2 mutations who present with ASD and other neuropsychiatric disorders (Strauss et al., 2006; Friedman et al., 2007). Notably, the perforated synapses present higher densities of glutamate receptors compared to non-perforated synapses (Lamprecht & LeDoux, 2004). This apparent discrepancy between increased expression of several AMPA-R and NMDA-R subunits and the decrease in the length of segmented PSDs of perforated synapses suggests that a significant proportion of these receptors might be located at extrasynaptic sites.

Abnormal Expression of Glutamate Receptor Subunits in the Prefrontal Cortex (PFC) of Cntnap2 Mutant Mice:

Based on the observation of the altered excitation/inhibition balance and abnormal structural synaptic plasticity in the cortex of Cntnap2 mutant mice, we set out to characterize the glutamate receptor subunit profiles in these mice. Therefore, we carried out immunoblot analysis of prefrontal cortex (PFC) homogenates from adult Cntnap2^{-/-}, Cntnap2^{+/-} and WT mice at 3-5 months and 9-11 months of age (n=5-6/group; FIG. 18A-B). We found that both Cntnap2^{-/-} and Cntnap2^{+/-} mice demonstrated abnormal ionotropic glutamate receptor (iGluR) subunit expression profiles, involving amino-3-hydroxy-5-methyl-4-isoxazolepropionic acid receptors (AMPA-Rs) and N-methyl-D-aspartate receptors (NMDARs). At 3-5 months, both Cntnap2^{-/-} and Cntnap2^{+/-} mice demonstrated significantly higher GluR1 (P<0.001 for Cntnap2^{+/-} and P<0.0001 for Cntnap2^{-/-}) and lower GluR2 expression (P<0.001), when compared to WT mice. Similarly, the GluR1 to GluR2 ratios were significantly higher in both genotypes (P<0.001 for Cntnap2^{+/-} and P<0.0001 for Cntnap2^{-/-}), suggesting increased Ca²⁺ permeability of AMPA-Rs. This observation is consistent with the increase in amplitude of sEPSCs that we found in the cortex of Cntnap2^{-/-} mutant mice. The Cntnap2^{-/-} mice also demonstrated significantly higher NR1 (P<0.01) and NR2B (P<0.0001) expression, as well as higher NR2B to NR2A ratios (p<0.0001) compared to WT mice. In contrast, Cntnap2^{+/-} mice demonstrated normal NR1 and NR2B levels and no significant change in the

NR2B to NR2A ratio (P>0.05), despite relatively lower NR2A expression (P<0.05).

Interestingly, this pattern of abnormal AMPA-R and NMDA-R subunit expression is consistent with an immature hyperexcitable pyramidal cell phenotype (Talos et al., 2006; Jantzie et al., 2013) and has also been described in post-mortem brain tissue from patients with ASD without a seizure disorder (Dilsiz et al., SfN Abstract 2012). The subsequent group I metabotropic glutamate receptors (mGluRs) analysis also revealed significant differences between genotypes (FIG. 18A-B). The Cntnap2^{-/-} mice showed upregulation of both mGluR1 (P<0.0001) and mGluR5 (P<0.001), while there was a selective increase in mGluR1 (P<0.05) in the Cntnap2^{+/-} littermates. As augmented signaling through group I mGluRs can lead to robust long-lasting spine shrinkage and elimination (Ramiro-Cortés & Israely, 2013) this finding might explain the increased spine elimination rates we observed in these mice.

In addition to the genotype-specific differences, we also found several significant changes with progressing age. Notably, at 9-11 months of age (FIG. 18A-B) both Cntnap2^{-/-} and Cntnap2^{+/-} mice demonstrated normal levels of GluR1 and mGluR1. However, there was a persistent decrease in GluR2, which was more pronounced in Cntnap2^{-/-} (P<0.05 for Cntnap2^{+/-} and P<0.0001 for Cntnap2^{-/-}), leading to significantly higher GluR1:GluR2 ratios in this group (P<0.0001). Older Cntnap2^{-/-} animals also demonstrated decreased NR2A (P<0.05)

expression, higher NR1 (P<0.0001), NR2B (P<0.01), NR2B:NR2A ratios (P<0.0001) and elevated mGluR5 (P<0.001), when compared to WT controls. Cntnap2^{+/-} mice showed selective increased NR1 (P<0.05) and NR2B (P<0.01) expression at 9-11 months of age.

Interestingly, the AMPA-R to NMDA-R ratios (i.e., GluR1:NR1 ratios) presented a dramatic reduction with age in both genotypes, although the difference was not statistically significant. Nevertheless, as AMPA-Rs are regarded as the major determinant of synaptic strength and plasticity, this shift in receptor sub-type composition at the glutamatergic synapses over time may significantly impact both synaptic plasticity and cognitive function in these mice.

Overactivation of the mTOR Pathway in Cntnap2 Mutant Mice:

Given the significant enlargement of cortical layer V pyramidal neurons in Cntnap2 mutant mice, and the fact that the mTOR kinase represents a key regulator of cell size (Lloyd, 2013), we sought to determine whether overactivation of this pathway might be responsible for this change. To address this, we used immunoblot analysis to quantify the expression levels of phosphorylated ribosomal

protein S6 (pS6), a marker of mTOR activity, in prefrontal cortex (PFC) homogenates from 3-5 mo and 9-11 mo old Cntnap2 mutant mice and WT littermates (FIG. 17F). The younger Cntnap2^{-/-} mice demonstrated significantly higher pS6 levels (P<0.001), which increased further with age (P<0.0001). The progressive age-dependent increase in pS6 expression was
even more dramatic in the Cntnap2^{+/-} mice, where pS6 rose from levels comparable to controls at 3-5 months of age to expression levels that were approximately 3-fold higher relative to WT at 9-11 moths (P<0.0001). This suggests that mTOR overactivation is present in younger mutant mice and increases dramatically with age. This is the first report of an effect of Cntnap2 deficiency on mTOR signaling. Since phosphorylation of S6 is directly associated with regulating cell size (Ruvinsky & Meyuhas, 2006), this finding is consistent with the increased pyramidal cell size in Cntnap2 mutant mice.

Enlarged Pyramidal and Dysplastic Cells with Differential Expression of Cell-Specific Markers and Increased pS6:

We examined whether the cellular phenotype identified in the Cntnap2 mutant mice involving enlarged pyramidal cells throughout the cortex is also evident in temporal cortex tissue surgically removed due to debilitating seizures from individuals with homozygous CNTNAP2 mutations who were also diagnosed with ASD. Cresyl violet (CV) staining, performed in three subjects and 3 age-matched controls, revealed a

pattern of diffusely distributed similarly enlarged cells, which appear to have intact morphological characteristics of pyramidal cells (FIG. 19A). To confirm that overactivation of the mTOR pathway accompanies this cellular phenotype, we carried out co-staining for CV and pS6 (blue and brown respectively, FIG. 19A). This analysis demonstrated strong pS6 staining in the enlarged pyramidal cells throughout the cortex in human mutation carriers, while cortical tissue from normal controls displayed only weak staining for pS6 (FIG. 19A).

As increased cell size due to overactivation of mTOR signaling in neurodevelopmental diseases such as Tuberous Sclerosis Complex (TSC) is consistently associated with mixed neuronal and glial lineage phenotypes (Sarnat 2013), we further examined the cellular pathology of CNTNAP2 mutation-carriers by utilizing the specific neuronal and glial maturational markers NeuN, MAP-2, nonphosphorylated neurofilament SMI311 and vimentin.

In addition to the previously identified enlarged pyramidal cells, we also observed that CNTNAP2 mutation is associated with altered neuronal differentiation. These undifferentiated enlarged cells were immunopositive for the neuronal marker SMI311, but also for vimentin, an intermediate filament typically expressed in neuroglial progenitor cells, including radial glia (Weissman et al., 2003), whereas the pyramidal neurons were only immunopositive for neuronal neurofilament marker SMI311 (FIG. 19A). The undifferentiated enlarged cells had a distinct rounded appearance and often presented two nuclei, resembling the TSC giant cells. The NeuN and MAP-2 markers, which indicate the cellular commitment to the neuronal lineage, were exclusively expressed in dysplastic neurons, not in “giant cells”, which again is reminiscent of what is found in cortical tubers in brain tissue from patients with TSC (Tabs et al., 2008).

Decreased Length of Perforated PSDs:

Next, we examined whether human subjects with homozygous CNTNAP2 mutations harbor a similar synaptic pathology (i.e., a selective reduction in length of the perforated PSDs) as mutant mice. We examined perforated PSDs in cortical layer I of 3 subjects and compared them with 3 age- and gender-matched controls. We observed a significant reduction in the length of perforated PSDs of CNTNAP2 mutation carriers (a 22% reduction; P=0.02; FIG. 19B) compared with the normal controls.

A selective decrease in the length of perforated PSDs can reflect impairments in long-term potentiation (LTP) induction and learning (Buchs & Muller, 1996; Lamprecht & LeDoux, 2004), both of which have been associated with mTOR overactivation (Hoeffer & Klann, 2010). Thus, this synaptic deficit likely represents one of the structural correlates of the severe cognitive deficits observed in all these three CNTNAP2 mutation carriers.

Altered Glutamate Receptor Subunit Expression Profile:

Finally, we evaluated cortical tissue from patients with CNTNAP2 mutations for patterns of abnormal expression of select iGluR and mGluR subunits in cortical layer V pyramidal neurons in patients (n=3) versus age and gender-matched controls (n=3). Overall, we observed a similar pattern as in the Cntnap2 mutant mice, further pointing to the validity of the Cntnap2 mutant mice in capturing key brain

alterations present in human subjects with CNTNAP2 mutations who suffer from ASD. We found that in the mutation carriers GluR1 and mGluR5 were highly expressed in the majority of pyramidal neurons, while all these subunits showed low expression in the controls (FIG. 19C).

In addition, NR2B was also increased, especially in the dendrites, while GluR2 was undetectable in most neurons from patients, but highly expressed in the control neurons. These receptor profiles are consistent with altered excitation/inhibition balance due to dysregulated mTOR signaling (Bateup et al., 2013). In addition, these changes suggest significant alterations of synaptic plasticity in these patients, due to both increased Ca²⁺ influx through AMPA-R and NMDA-R with altered subunit composition, as well as upregulated mGluR5-dependent signaling.

Rescue of asd-Related Core Deficits in Cntnap2 Mutant Mice Utilizing WYE125132, a Highly Potent and Selective mTor Inhibitor:

Rapamycin, the first mTOR inhibitor described, has been consistently utilized to achieve

repression of the mTOR signaling pathway in mouse models of ASD (Delorme et al., 2013). However, the fact that rapamycin is a partial mTOR inhibitor acting through allosteric inhibition of the mTORC1—but not the mTORC2—complex, while mTORC1 is also known to have some rapamycin-resistant activity raises the fundamental question as to whether rapamycin can indeed lead to a robust and sustained inhibition of mTOR (Guertin & Sabatini, 2009).

Furthermore, there is a negative feedback mechanism downstream of mTORC1 (Huang 2009), where mTORC1 inhibition by rapamycin leads to a net enhancement of PI3K-AKT pathway (Guertin & Sabatini, 2009). Because of these potential limitations with rapamycin and similar rapalogs, we decided to use WYE125132, a highly potent, ATP-competitive, and specific new generation mTOR kinase inhibitor, which targets the catalytic site, inhibits both mTORC1 and mTORC2 and has a good bioavailability profile. WYE125132 is therefore capable of leading to strong and sustained mTOR inhibition in vivo (Yu et al., 2010).

Correction of Enlarged Pyramidal Cell Phenotype and Increased Phosporylation of S6:

We examined whether we can reverse the dramatic enlargement of the pyramidal cell somata by targeting the mTOR overactivation. To this end, mice were gavaged daily with WYE125132 from P12 until P33-36 after which their brains were harvested for further analysis. A 3-week treatment with WYE125132 led to a complete reversal of the pyramidal cell enlargement phenotype in both Cntnap2^{+/-} and Cntnap2^{-/-} mutant mice (P<0.0001). There was a small but significant difference in pyramidal cell size between WT and Cntnap2 mutant mice treated with WYE125132 (p<0.001; Kruskal-Wallis one-way ANOVA) (FIG. 20A, C,D). However, no difference was observed between WT mice treated with vehicle versus the compound, indicating that WYE125132 has no effect on soma size in WT animals (FIG. 20A). In concordance with the normalization of pyramidal cell size in the cortex of Cntnap2 mutant mice, we also found that treatment with WYE125132 reverses phosphorylation of S6 to levels observed in WT littermates (P<0.001; FIG. 20B).

Reversal of Glutamate Receptor Subunit Alterations:

We also evaluated whether treatment with WYE126132 reverses the alterations in the expression of the glutamate receptor subunits found in Cntnap2 mutant mice. Mice aged 5-8 months were treated for consecutive days. Immunoblot analysis of PFC homogenates from vehicle- and WYE126132-treated Cntnap2^{-/-}, Cntnap2^{+/-} and WT mice showed a complete normalization of all glutamate receptor subunits that were found altered in untreated Cntnap2 mutants (FIG. 20E), which

included GluR1, GluR2, NR1, NR2A, NR2B and mGluR5. At the same time, we observed no effects of WYE126132 treatment in the WT animals. Thus, the robust alterations in glutamate receptor subunit expression seen in Cntnap2 mutant mice are entirely reversed upon treatment with WYE125132.

Reversal of Abnormal Neuronal Network Activity and Plasticity:

We investigated whether we could reverse the impairment in neuronal network activity and plasticity by targeting the overactivation of the mTOR pathway with WYE125132. Adult animals (ages 3-7 months) were gavaged for 14 days with either WYE125132 or vehicle. Whole-cell recordings were made from layer V pyramidal neurons in the auditory cortex, and synaptic events were evoked with an extracellular stimulation electrode placed <150 μm from the soma of the

recorded cell. We first asked whether WYE125132 treatment restores the relationship between excitatory and inhibitory synaptic strength to WT levels. We assessed this relation over multiple inputs onto the recorded postsynaptic neuron from different regions of the cortical brain slice, by systematically varying the intensity of extracellular stimulation, such that higher intensities evoke responses from larger subpopulations of presynaptic inputs (House et al., 2011). This relation was defined as the linear correlation coefficient between excitation and inhibition at each different intensity value. We found that there was a high correlation between excitation and inhibition in neurons recorded from WT animals and Cntnap2 mutant animals treated with WYE125132, but a significantly lower correlation in mutant animals treated with vehicle (WYE125132-treated mutant mice, r: 0.43±0.08, n=12; vehicle-treated mutant mice, r: 0.09±0.13, n=17, P<0.04 compared to WYE125132-treated mutant mice, Student's two-tailed t-test; WT littermates, r: 0.59±0.06, n=9; FIG. 21A).

Since both the specific decrease in length of the perforated PSDs as well as a persistent elevation of mGluR5 have been associated with cognitive impairment and LTP (Lamprecht & LeDoux, 2004; Neyman & Manahan-Vaughan, 2008), we also examined the impact of WYE125132 treatment on the induction of long-term plasticity. After recording baseline synaptic responses for several minutes, we attempted to induce spike-timingdependent LTP by repetitively pairing single EPSPs with single postsynaptic action potentials (Bi et al., 1998; Feldman, 2000; Froemke et al., 2006), and then monitoring synaptic strength for 10 minutes thereafter. Spike pairing induced significant LTP in WT animals, but failed to potentiate synaptic strength in cells recorded from mutant animals treated with vehicle. However, spike pairing successfully induced LTP in neurons from mutant animals given WYE125132 (WYE125132-treated mutant mice: 45.4±16.4% increase, n=8; vehicle-treated mutant mice: −4.4±13.3% decrease, n=9, p<0.03 compared to WYE125132-treated mutant mice, Student's two-tailed t-test; WT littermates: 33.8±11.4% increase, n=6; FIG. 21B).

Reversal of Abnormalities in Dendritic Spine Dynamics:

To examine whether we could reverse the abnormalities in dendritic spine dynamics in the frontal association cortex, we gavaged the mice daily from P12 to P32 with WYE125132 versus vehicle. Interestingly, the abnormally high rates of spine elimination in Cntnap2 mutant mice were reduced back to levels seen in the WT littermates (Cntnap2^{-/-} mice: 9.5%±1.6%; Cntnap2^{+/-} mice: 8.5%±2.0%, P>0.05, FIG. 21C). These data suggest that WYE125132 is indeed able to restore normal dendritic spine plasticity in both Cntnap2^{+/-} and Cntnap2^{-/-} mutant mice.

Rescue of Social Interaction Deficit and Stereotyped Behaviors:

Guided by human genetic studies, which suggest that most individuals with a mutation or CNV in the CNTNAP2 gene are heterozygously affected and because our behavioral analysis showed that Cntnap2^{+/-} mice demonstrate social deficits, we focused on Cntnap2^{+/-} mice for our social behavior rescue experiments. Mutant mice were treated with WYE125132 or vehicle by daily gavaging. Consistent with our findings in untreated mice, Cntnap2^{+/-} mice treated with vehicle demonstrated significantly reduced social interactions during the first minute of testing (FIG. 21D; P<0.05), while Cntnap2^{+/-} mice treated with WYE125132 demonstrated a reversal of the social interaction deficits in the first minute of testing compared with Cntnap2^{+/-} mice that received vehicle (P<0.01; FIG. 21D). Overall, our findings suggest that WYE125132 increases the time that Cntnap2 mutant mice spend sniffing the stimulus mouse in the very first part of

the test whereas it has no such effect in WT littermates. Social inhibition in the first minute is indicative of a reduced tendency to approach novel social stimuli or increased social anxiety (Curley et al., 2009). Interestingly, a specific reduction in preference for social novelty has also been described in the Fragile X Syndrome mouse model where overactivation of the mTOR pathway is also present (Heitzer et al., 2013) and a common variant in CNTNAP2 has been associated with social anxiety-related traits in human subjects (Stein et al., 2011).

Furthermore, when examining the entire 10-minute period, vehicle-treated Cntnap2^{+/-} mice demonstrated a significant reduction in the number of social interactions compared with WT littermates that received vehicle (FIG. 21D; P<0.05), and this was rescued by treatment with WYE125132 (FIG. 21D; P<0.05). When examining grooming, a stereotyped repetitive behavior associated with ASD in mice (Silverman et al., 2010), maximum grooming bout duration also increased significantly in Cntnap2^{+/-} mutant mice treated with WYE125132 compared with vehicle (P<0.05; FIG. 21D). Together, these findings indicate that treatment with WYE125132 reverses social behavior deficits while it also leads to less fragmentary repetitive social and non-social behaviors.

Rescue of Cognitive Deficit: Novel Object Recognition:

Human subjects carrying CNTNAP2 mutations (Strauss et al., 2006; Friedman et al., 2007), as well as Cntnap2^{-/-} mice, exhibit cognitive deficits (Penagarikano et al., 2011). We examined whether we could reverse cognitive deficits in Cntnap2 mutant mice. We subjected Cntnap2 mutant mice to the Novel Object Recognition (NOR) test, a behavioral task that requires precise cognitive control. When looking at Cntnap2^{+/-}, Cntnap2^{-/-}, and WT mice, we found a main effect of genotype (P=0.03) and a genotype x drug interaction (Two-Way ANOVA; P=0.04). Post-test analyses showed a significant difference between vehicle-treated WT mice and vehicle-treated Cntnap2^{-/-} mice (P<0.05; FIG. 21E) as well as a rescue of this phenotype in WYE125132-treated Cntnap2^{-/-} mice (P<0.05; FIG. 21E). Interestingly, we found that vehicle-treated Cntnap2^{-/-} mice exhibit enhanced preference for the familiar object in the NOR task, although this was not statistically significant. Brain-specific disruption of FK506-binding protein 12 (FKBP12), a regulator of mTOR, has been shown to also cause a similar preference for the familiar object over the novel object in mice (Hoeffer et al., 2008). This could indicate that Cntnap2^{-/-} mice do not remember the familiar object, are aversive to novelty, are preservative for the familiar object, or have an inability to inhibit responding to the familiar object (Hoeffer et al., 2008;

Bhattacharya et al., 2012). Since NOR is a hippocampus- and entorhinal cortex-dependent task, this suggests that frontal- and temporal cortex-dependent sensory information processing is defective in Cntnap2^{-/-} mice. These findings suggest that WYE125132 can effectively target the cognitive deficits in Cntnap2 mutant mice.

### 6.3 DISCUSSION

Experiments described in this section were designed to evaluate how mutations in CNTNAP2, a strong risk factor for ASD and a number of neuropsychiatric disorders, affects the structure and function of cortical neural circuits in order to systematically test whether these effects are reversible. In summary, this analysis led to a number of key findings: First, we showed that mutations in CNTNAP2 lead to overactivation of the mTOR signaling. This is, to our knowledge, the first report of mTOR signaling dysfunction in a non-syndromic form of ASD as opposed to other syndromal ASD presentations, such as TSC, FXS, and PTEN mutations. Second, our extensive and in-depth phenotypic characterization allowed us to describe CNTNAP2-related dysfunction at different

levels of neuronal organization. Third, our access to human brain tissue of patients with ASD and CNTNAP2 mutations and our demonstration that signature molecular, synaptic, and cellular phenotypes are present in the brains of both Cntnap2 mutant mice and patients, establish one of the strongest cases for face validity in a genetic mouse model of ASD and unequivocally confirm the presence of mTOR overactivation in human subjects with CNTNAP2 mutations. Fourth, utilizing a potent and highly selective mTOR kinase inhibitor, we were able to rescue many core molecular, synaptic, cellular, neurocircuitry, and behavioral phenotypes in the Cntnap2 mutant mice confirming that the ASD-related pathophysiology in mutant mice is driven to a large extent by overactive mTOR signaling. This is one of the first efforts for novel drug discovery in non-syndromic ASD based on a gene target unequivocally linked to disease risk.

Our finding is particularly relevant in the emerging field of personalized medicine where simple genetic tests will allow for targeted treatments for subsets of patients. Using independent behavioral tests, we corroborated and expanded on previous findings (Penagarikano et al., 2011) that Cntnap2 deficient mice have deficits in social interaction, grooming, cognition and a lowered seizure threshold as assessed by utilizing a pilocarpine-induction paradigm. These behavioral phenotypes are consistent with the neuropsychiatric phenotypes seen in human CNTNAP2 mutation carriers. Examination of the neuroanatomy of the Cntnap2 mutants on a mesoscopic level, utilizing high-resolution MM assessment of volumetric changes ex vivo, demonstrated that

brain structure in Cntnap2 mutant mice is grossly normal, except for a reduction in the relative volume of the occipital cortex. Interestingly, a recent study reported on a similar volumetric deficit in human subjects who harbor a SNP variant of CNTNAP2, which has been associated with ASD in three independent studies (Tan et al., 2010). Grossly normal neuroanatomy is consistent with histological analyses of brains from Cntnap2 mutant mice, which found no gross morphological changes in the brain structure of mutant animals by conventional staining techniques (i.e., cresyl violet staining) (Poliak et al., 2003).

Uptake of the PET tracer FDG has also specifically been correlated with mTOR overactivation and it has been suggested that FDG-PET imaging could be utilized as a biomarker in clinical trials utilizing mTOR inhibitors (Thomas et al., 2006). FDG-microPET/MRI imaging in Cntnap2 mutant mice demonstrated that mutant mice exhibit a pattern of metabolic activity alterations predominated by cortical and subcortical hypermetabolism. Previous studies have demonstrated hypermetabolism in specific brain regions in human subjects with disorders involving mTOR overactivation and are associated with ASD, such as tuberous sclerosis complex (TSC) (Asano et al., 2001) and Fragile X Syndrome (FXS) (Schapiro et al., 1995). These brain regions include cortex, thalamus, and cerebellum, all of which demonstrate increased metabolism in the Cntnap2 mutants. Moreover, focal hypermetabolism in the cerebellum and caudate nucleus correlates with stereotyped behavior, impaired social interaction and communication deficits in patients with TSC who are also diagnosed with ASD

(Asano et al., 2001). Hypermetabolism throughout the brain found by FDG-PET imaging has been associated with frequent or continuous seizure activity (Meltzer et al., 2000), similar to what we observe in the Cntnap2 mice using pilocarpine seizure induction.

Guided by these findings, we characterized network activity and plasticity of cortical networks. We identified changes in both amplitude and frequency of sEPSCs as well as amplitude of sIPSCs in Cntnap2 mutants, indicative of an imbalance in the excitation/inhibition balance as one of the neural substrates of the CNTNAP2-associated cortical hypermetabolism and epileptic activity. mTOR overactivation has been shown to lead to an altered balance of

excitatory and inhibitory synaptic transmission which, in turn, has been associated with hippocampal hyperexcitability (Bateup et al., 2013) and impaired cellular information processing and behavioral deficits consistent with ASD-associated phenotypes (Yizhar et al., 2011; Gkogkas et al., 2013). Examination of the frontal cortex utilizing in vivo transcranial twophoton microscopy revealed an increase in elimination and decrease in formation of dendritic spines. Consistently, it was recently demonstrated that RNAi-mediated knockdown of Cntnap2 leads to abnormalities in spine development in pyramidal neurons (Anderson et al., 2012).
Accompanying these abnormalities in network activity and plasticity were also robust changes in the ultrastructure and molecular composition of the cortical excitatory synapses. Electron microscopy of the frontal cortex revealed a highly selective decrease in length of perforated PSDs. Perforated synapses are characterized by a discontinuity in the postsynaptic density, resulting in a hole, a slit or a complete segmentation of the postsynaptic density plate. This is thought to reflect a structural correlate of enhanced efficacy of synaptic transmission, which is believed to underlie learning (Morrison & Baxter, 2012). LTP induction and learning lead to a dramatic increase in both the number and size of perforated PSDs (Buchs & Muller, 1996; Lamprecht & LeDoux, 2004) and glutamate receptor immunoreactivity in perforated PSDs has been shown to be significantly higher than in non-perforated PSDs (AMPA-R>NMDA-R) (Lamprecht & LeDoux, 2004). A highly selective reduction in size/length of perforated PSDs (but not non-perforated PSDs) in hippocampal axospinous synapses has been found to correlate with age-related cognitive impairment (e.g., spatial learning) (Nicholson et al., 2004). Decrease in length of perforated PSDs reflects a disruption in synaptic complexity and the capacity for encoding and retrieving complex information. As such, these smaller perforated synapses become less efficient or postsynaptically silenced in aged, learning-impaired animals (Nicholson et al., 2004). In that respect, decrease in length of the perforated PSDs may be a structural correlate of the cognitive deficits and other neuropsychiatric symptoms seen in mutant mice and humans with CNTNAP2 mutations.

We found that excitatory synapses in the frontal cortex are further characterized by a specific pattern of alterations in glutamate receptor subunit composition, including an increase in expression of GluR1, and NR1, NR2B and mGluR5 while GluR2 and NR2A were both decreased in Cntnap2 mutants, highly suggestive of synaptic immaturity (Talos et al., 2006; Jantzie 2013). While an increase in mGluR5 signaling has been associated with overactivation of mTOR, altered protein translation, increased spine elimination, LTP impairment, cognitive deficits, and syndromal ASD (Michalon et al., 2012; Wilson et al., 2007; Lohith et al., 2013; Anagnostou et al., 2012; Ramiro-Cortes & Israely, 2013), a decrease in GluR2 has been linked to impaired AMPA-R assembly and LTP, cognitive deficits, and a decrease in size of perforated PSDs (Medvedev et al., 2008). It remains unknown how the observed changes in synaptic activity, plasticity, ultrastructure and molecular composition are related. However, it is worth noting that an increased excitation/inhibition ratio has been associated with abnormal

critical window plasticity (Ma et al., 2013), potentially leading to a state of immature cortical neurocircuitry as evident in the abnormal spine dynamics and immature hyperexcitable glutamate receptor subunit profile.

Examination of the cellular morphology in the cortex of Cntnap2 mutant mice was particularly instructive. Mutant mice exhibited a dramatic cellular phenotype characterized by enlargement of pyramidal cells throughout all layers of the cortex. The striking finding of enlarged pyramidal cells throughout the cortex led us to identify overactivation of the mTOR pathway as the disease mechanism of CNTNAP2-associated ASD by demonstrating a robust increase in phosphorylation of the S6 protein in the cortex of Cntnap2 mutant mice. A

mechanistic link between Cntnap2 deficiency and the mTOR pathway, which could explain the overactivation of the mTOR signaling in mutant mice, remains to be determined. One possible link is altered semaphorin signaling via the sema3A receptor, which forms a complex with Cntnap2 and TAG-1 and can activate the mTOR pathway when disrupted (Dang et al., 2012).

It remains to be determined whether this or other signaling alterations are responsible for the abnormalities in mTOR signaling in Cntnap2 mutant mice. Importantly, we showed that the striking cellular phenotype involving enlarged pyramidal cells seen in Cntnap2 mutant mice is recapitulated in human cortical brain tissue from patients with homozygous CNTNAP2 mutations, while we also identified the presence of undifferentiated “giant cells”. Both of these cell types are diffusely present throughout the cortex and demonstrate increased phosphorylation of intracellular S6. This dramatic cellular phenotype confirms the presence of overactivation of the mTOR pathway in the human brain

tissue and is also consistent with an immaturity of the cortical neurocircuitry. Compared with matching controls, we were able to demonstrate changes in both glutamate receptor subunits and perforated PSD structure in patients harboring CNTNAP2 mutations identical to the ones in the mouse model. Interestingly, the immature profile of expression of the glutamate receptor subunits has recently been described in postmortem brain tissue from human subjects diagnosed with ASD who do not suffer from seizures (Dilsiz et al., SfN abstract 2012).

Moreover, the pattern observed here is similar to one described in cortical brain tissue from patients with TSC, where pyramidal cells in tubers demonstrate an increase in GluR1 and NR2B expression and a relative GluR2 deficiency (Talos et al., 2008). This suggests that the neuropsychiatric manifestations in both conditions involve overactivation of the mTOR pathway

and may be driven by a comparable form of defective glutamatergic signaling. Because of the established face validity of the Cntnap2 mouse model, we sought to identify a compound with therapeutic potential to prevent or reverse all disease-associated neural alterations in human subjects with mental illness who harbor CNTNAP2 mutations. Our in vivo treatment experiments with WYE125132 demonstrate that all core disease phenotypes on the molecular, synaptic, cellular, neurocircuitry, and behavioral levels are indeed preventable and/or reversible in Cntnap2 mutant mice, which strongly suggest that WYE125132, and other similar mTOR kinase inhibitors, will have a comparable therapeutic potential for human subjects with neuropsychiatric disorders who harbor CNTNAP2 mutations.

It is worth noting here that in addition to an association with ASD and epilepsy, CNTNAP2 mutations have also been associated with SCZ in GWAS (Wang et al., 2010) and CNV studies (Friedman et al., 2008). Although analysis of SCZ-related behaviors and treatment reversal studies remain to be done in the Cntnap2 mouse model, many of the underlying neural substrates studied here are likely shared among these psychiatric disorders. This is further supported by recent evidence that a genetic overlap exists between ASD and SCZ (Gilman et al., 2012) as well as by identification of de novo mutations in MTOR and other members of this signaling pathway in patients with SCZ (Xu et al., 2012). Therefore, our results may also have important implications in the treatment of SCZ and other

neuropsychiatric disorders. Moreover, both the glutamate receptor subunit expression profile and pattern of FDG-PET/MRI findings suggest the possibility of utilizing functional imaging modalities to identify biomarkers, which can be utilized in clinical treatment trials.

Mutations in the CNTNAP2 gene may only account for a small fraction of cases, but here we show that the gene is involved in signaling pathways previously implicated in syndromic ASD and therefore having possibly far-reaching effects. Therefore, the study of CNTNAP2 has the potential for a more generalized understanding of disease mechanisms and therapies. Our findings furthered our knowledge of the cellular and neurophysiological consequences of CNTNAP2 deletions and allowed us to identify compounds targeting the mechanisms that lead from gene mutation to disease. Targeting a known genetic variation enables the generation of reliable mouse models that closely mimic the risk alleles, therefore ensuring maximal translational validity.

### 9 EXAMPLE

### Effect of Other mTOR Pathway Inhibitors on S6 Phosphorylation in Mutant Mice

Mutant CNTNAP2 mice or wild-type mice (weighing an average of 25 g) were treated for 14 consecutive days with rapamycin (LC Systems) 3 mg/kg, Torin2 (Tocris, Liu et al. (2011, 2013)) 10 mg/kg, AZD2014 (Chemscene LLC) 10 mg/kg or vehicle, and at the conclusion of the study, the mice were sacrificed and cortical samples were evaluated for phosphorylated S6 by Western blot. In particular, frozen cortical samples were utilized for either membrane or whole cell protein extracts. Lysis buffer supplemented with Complete Mini Protease Inhibitor Cocktail Tablet (Roche, Germany) phenylmethanesulfonyl fluoride (1 mM), sodium orthovanadate (1 mM) and okadaic acid (0.1 mM) was used to homogenize the frozen samples. The protein levels were measured. 20 μg of membrane and whole cell protein preparations were loaded on SDS 4-20% polyacrylamide gradient gel and transferred to nitrocellulose membrane. The membranes were probed for pS6 (Ser235/236) (1:500, Cell Signaling), S6 (1:500, Cell Signaling), and actin (1:2000, Millipore). Following the primary antibody incubation, horseradish peroxidase conjugated anti-rabbit/anti-mouse IgG secondary antibodies (1:2000, Vector Laboratories) were used. Relative optical density was measured for each band using ImageJ software. Relative optical density measurements were then expressed as a percentage of the mean density of age-matched controls, and protein levels were compared using paired student's t-tests.

The results are shown in FIG. 22.

### 10. REFERENCES

Alarcón M, et al. Linkage, association, and gene-expression analyses identify CNTNAP2 as an autism-susceptibility gene. Am J Hum Genet 82:150-9 (2008).
Anagnostou, E. (2012). Translational medicine: Mice and men show the way. Nature 491, 196-7.
Anderson, G. R., Galfin, T., Xu, W., Aoto, J., Malenka, R. C., Südhof, T. C. (2012). Candidate autism gene screen identifies critical role for cell-adhesion molecule CASPR2 in dendritic arborization and spine development. Proc. Natl. Acad. Sci. USA. 109, 18120-5.
Anney, R., Klei, L., Pinto, D., Almeida, J., Bacchelli, E., Baird, G., Bolshakov, N., Bölte, S., Bolton, P. F., Bourgeron, T. (2012). Individual common variants exert weak effects on the risk for autism spectrum disorders. Hum. Mol. Genet. 21, 4781-92.
American Psychiatric Association. (2000). Diagnostic and statistical manual of mental disorders (4th ed., text rev.). Washington, DC: Author.
Arkin D E, et al. A common genetic variant in the neurexin superfamily member CNTNAP2 increases familial risk of autism. Am J Hum Genet 82:160-4 (2008).
Asano, E., Chugani, D. C., Muzik, O., Behen, M., Janisse, J., Rothermel, R., Mangner, T. J., Chakraborty, P. K., Chugani, H. T. (2001). Autism in tuberous sclerosis complex is related to both cortical and subcortical dysfunction. Neurology 57, 1269-77.
Bateup, H. S., Johnson, C. A., Denefrio, C. L., Saulnier, J. L., Kornacker, K., Sabatini, B. L. (2013). Excitatory/inhibitory synaptic imbalance leads to hippocampal hyperexcitability in mouse models of tuberous sclerosis. Neuron 78, 510-22.
Bhattacharya, A., Kaphzan, H., Alvarez-Dieppa, A. C., Murphy, J. P., Pierre, P., Klann, E. (2012). Genetic removal of p70 S6 kinase 1 corrects molecular, synaptic, and behavioral phenotypes in fragile X syndrome mice. Neuron 76, 325-37.
Bi, G. Q., Poo, M. M. (1998). Synaptic modifications in cultured hippocampal neurons: dependence on spike timing, synaptic strength, and postsynaptic cell type. J. Neurosci. 18, 10464-72.
Buchs, P. A., Muller, D. (1996). Induction of long-term potentiation is associated with major ultrastructural changes of activated synapses. Proc. Natl. Acad. Sci. USA. 93, 8040-5.
Buizer-Voskamp J E, et al. Genome-wide analysis shows increased frequency of copy number variation deletions in Dutch schizophrenia patients. Biol Psychiatry 70:655-62 (2011).
Curley, J. P., Jordan, E. R., Swaney, W. T., Izraelit, A., Kammel, S., Champagne, F. A. (2009). The meaning of weaning: influence of the weaning period on behavioral development in mice. Dev. Neurosci. 31, 318-31.
Dabell, M. P., Rosenfeld, J. A., Bader, P., Escobar, L. F., El-Khechen, D., Vallee, S. E., Dinulos, M. B., Curry, C., Fisher, J., Tervo, R., et al. (2013). Investigation of NRXN1 deletions: clinical and molecular characterization. Am. J. Med. Genet. A. 161A, 717-31.
Delorme, R., Ey, E., Toro, R., Leboyer, M., Gillberg, C., Bourgeron, T. (2013). Progress toward treatments for synaptic defects in autism. Nat. Med. 19, 685-94.
Dang, P., Smythe, E., Furley, A. J. (2012). TAG1 regulates the endocytic trafficking and signaling of the semaphorin3A receptor complex. J. Neurosci. 32, 10370-82.
Ehninger, D., Han, S., Shilyansky, C., Zhou, Y., Li, W., Kwiatkowsky, D. J., Ramesh V., Silva A. J. (2008). Reversal of learning deficits in a TSC2+/− mouse model of tuberous sclerosis. Nature Med. 14, 843-848.
Ehninger, D. and Silva, A. J. (2011). Rapamycin for treating tuberous sclerosis and autism spectrum disorders. Trends in Molecular Med. 17, 78-87.
Elia J, et al. Rare structural variants found in attention-deficit hyperactivity disorder are preferentially associated with neurodevelopmental genes. Mol Psychiatry 15:637-46 (2010).
Feldman, D. E. (2000). Timing-based LTP and LTD at vertical inputs to layer II/III pyramidal cells in rat barrel cortex. Neuron 27, 45-56.
Friedman J I, Vrijenhoek T, Markx S, et al. CNTNAP2 gene dosage variation is associated with schizophrenia and epilepsy. Mol Psychiatry 13:261-6 (2008).
Froemke, R. C., Tsay, I. A., Raad, M., Long, J. D., Dan, Y. (2006). Contribution of individual spikes in burst-induced long-term synaptic modification. J. Neurophysiol. 95, 1620-9.
Geschwind, D. H. (2009). Advances in autism. Annu. Rev. Med. 60, 367-80.
Gilman, S. R., Chang, J., Xu, B., Bawa, T. S., Gogos, J. A., Karayiorgou, M., Vitkup, D. (2012). Diverse types of genetic variation converge on functional gene networks involved in schizophrenia. Nat. Neurosci. 14, 1723-8.
Gkogkas, C. G., Khoutorsky, A., Ran, I., Rampakakis, E., Nevarko, T., Weatherill, D. B., Vasuta, C., Yee, S., Truitt, M., Dallaire, P., et al. (2013). Autism-related deficits via dysregulated eIF4E dependent translational control. Nature 493, 371-7.
Goto J, et al. Regulable neural progenitor-specific Tsc1 loss yields giant cells with organellar dysfunction in a model of tuberous sclerosis complex. Proc Natl Acad Sci USA 108:1070-9 (2011).
Grutzendler, J., Kasthuri, N., Gan, W. B. (2002). Long-term dendritic spine stability in the adult cortex. 420, 812-6.
Guertin, D. A., Sabatini, D. M. (2009). The pharmacology of mTOR inhibition. Sci. Signal. 2, pe24.
Heitzer, A. M., Roth, A. K., Nawrocki, L., Wrenn, C. C., Valdovinos, M. G. (2013). Brief report: Altered social behavior in isolation-reared Fmr1 knockout mice. J. Autism. Dev. Disord. 43, 1452-8.
Hering H & Sheng M. Dendritic spines: structure, dynamics, and regulation. Nat Rev Neurosci 2:880-8 (2001).
Hoeffer, C. A., Tang, W., Wong, H., Santillan, A., Patterson, R. J., Martinez, L. A., Tejada-Simon, M. V., Paylor, R., Hamilton, S. L., Klann, E. (2008). Removal of FKBP12 enhances mTORRaptor interactions, LTP, memory, and perseverative/repetitive behavior. Neuron 60, 832-45.
Hoeffer, C. A., Klann, E. (2010). mTOR signaling: at the crossroads of plasticity, memory and disease. Trends Neurosci. 33, 67-75.
Horresh, I., Poliak, S., Grant, S., Bredt, D., Rasband, M. N., Peles, E. (2008). Multiple molecular interactions determine the clustering of Caspr2 and Kv1 channels in myelinated axons. J. Neurosci. 28, 14213-22.
House, D. R., Elstrott, J., Koh, E., Chung, J., Feldman, D. E. (2011). Parallel regulation of feedforward inhibition and excitation during whisker map plasticity. Neuron 72, 819-31.
Huang, J., Manning, B. D. (2009). A complex interplay between Akt, TSC2 and the two mTOR complexes. Biochem. Soc. Trans. 37, 217-22.
Jantzie, L. L., Talos, D. M., Jackson, M. C., Park, H. K., Graham, D. A., Lechpammer, M., Folkerth, R. D., Volpe, J. J., Jensen, F. E. (2013). Developmental Expression of N-Methyl-DAspartate (NMDA) Receptor Subunits in Human White and Gray Matter: Potential Mechanism of Increased Vulnerability in the Immature Brain Cereb. Cortex [Epub ahead of print].
Judenhofer M S, et al. Simultaneous PET-MRI: a new approach for functional and morphological imaging. Nat Med 14: 459-65 (2008).
   of Increased Vulnerability in the Immature Brain Cereb. Cortex [Epub ahead of print].
Kwakye, L. D., Foss-Feig, J. H., Cascio, C. J., Stone, W. L., Wallace, M. T. (2011). Altered auditory and multisensory temporal processing in autism spectrum disorders. Front. Integr. Neurosci. 4, 129.
Lai, C. S., Franke, T. F., Gan, W. B. (2012). Opposite effects of fear conditioning and extinction on dendritic spine remodelling. Nature 483, 87-91.
Laplante, M., Sabatini, D. M. (2012). mTOR signaling in growth control and disease. Cell 149, 274-93.
Lamprecht, R., LeDoux, J. (2004). Structural plasticity and memory. Nat. Rev. Neurosci. 5, 45-54.
Liu et al. (2011) Discovery of 9-(6-aminopyridin-3-yl)-1-(3-(trifluoromethyl)phenyl)benzo[h][1,6]naphthyridin-2(1H)-one (Torin2) as a potent, selective, and orally available mammalian target of rapamycin (mTOR) inhibitor for treatment of cancer. J. Med. Chem. 54:1473-1480.
Liu et al. (2013). Characterization of Torin2, an ATP-competitor of mTOR, ATM and ATR. Cancer Res. 73(8):2574-2586.
Lloyd, A. C. (2013). The regulation of cell size. Cell 154, 1194-205.
Lo-Castro, A., Curatolo, P. (2013). Epilepsy associated with autism and attention deficit hyperactivity disorder: Is there a genetic link? Brain Dev. [Epub ahead of print].
Lohith, T. G., Osterweil, E. K., Fujita, M., Jenko, K. J., Bear, M. F., Innis, R. B. (2013). Is metabotropic glutamate receptor 5 upregulated in prefrontal cortex in fragile X syndrome? Mol. Autism. 4, 15.
Luat A F, et al. Neuroimaging in tuberous sclerosis complex. Curr Opin Neurol 20:142-50 (2007).
Ma, W. P., Li, Y. T., Tao, H, W. (2013). Downregulation of cortical inhibition mediates ocular dominance plasticity during the critical period. J. Neurosci. 33, 11276-80.
Medvedev, N. I., Rodriguez-Arellano, J. J., Popov, V. I., Davies, H. A., Tigaret, C. M., Schoepfer, R., Stewart, M. G. (2008). The glutamate receptor 2 subunit controls post-synaptic density complexity and spine shape in the dentate gyms. Eur. J. Neurosci. 27, 315-25.
Mefford, H. C., Muhle, H., Ostertag, P., von Spiczak, S., Buysse, K., Baker, C., Franke, A., Malafosse, A., Genton, P., Thomas, P., Gurnett, C. A., Schreiber, S., Bassuk, A. G., Guipponi, M., Stephani, U., Helbig, I., Eichler, E. E. (2010). Genome-wide copy number variation in epilepsy: novel susceptibility loci in idiopathic generalized and focal epilepsies. PLoS Genet. 6, 5.
Meltzer, C. C., Adelson, P. D., Brenner, R. P., Crumrine, P. K., Van Cott, A., Schiff, D. P., Townsend, D. W., Scheuer, M. L. (2000). Planned ictal FDG PET imaging for localization of extratemporal epileptic foci. Epilepsia 41, 193-200.
Michalon, A., Sidorov, M., Ballard, T. M., Ozmen, L., Spooren, W., Wettstein, J. G., Jaeschke, G., Bear, M. F., Lindemann, L. (2012). Chronic pharmacological mGlu5 inhibition corrects fragile X in adult mice. Neuron 74, 49-56.
Morrison, J. H., Baxter, M. G. (2012). The ageing cortical synapse: hallmarks and implications for cognitive decline. Nat. Rev. Neurosci. 13, 240-50.
Neyman, S., Manahan-Vaughan, D. (2008). Metabotropic glutamate receptor 1 (mGluR1) and 5 (mGluR5) regulate late phases of LTP and LTD in the hippocampal CA1 region in vitro. Eur. J. Neurosci. 27, 1345-52.
Nicholson D A, et al. Reduction in size of perforated postsynaptic densities in hippocampal axospinous synapses and age-related spatial learning impairments. J Neurosci 24:7648-53 (2004).
O'Roak, B. J., Deriziotis, P., Lee, C., Vives, L., Schwartz, J. J., Girirajan, S., Karakoc, E., Mackenzie, A. P., Ng, S. B., Baker, C., et al. (2011). Exome sequencing in sporadic autism spectrum disorders identifies severe de novo mutations. Nat. Genet. 43, 585-9.
O'Roak, B. J., Vives, L., Girirajan, S., Karakoc, E., Krumm, N., Coe, B. P., Levy, R., Ko, A., Lee, C., Smith, J. D., et al. (2012). Sporadic autism exomes reveal a highly interconnected protein network of de novo mutations. Nature 485, 246-50.
Pascu, J., Gispert, J. D., Michaelides, M., Thanos, P. K., Volkow, N. D., Vaquero, J. J., Soto Montenegro, M. L., Desco, M. (2009). Automated method for small-animal PET image registration with intrinsic validation. Mol. Imaging Biol. 11, 107-13.
Penagarikano O, et al. Absence of CNTNAP2 leads to epilepsy, neuronal migration abnormalities and core autism-related deficits. Cell 147:235-46 (2011).
Pike et al., (2013). “Optimization of potent and selective dual mTORC1 and mtORC2 inhibitors: the discovery of AZD8055 and AZD2014,” Bioorg. Med. Chem. Lett. 23:1212-1216.
Poliak, S., Gollan, L., Martinez, R., Custer, A., Einheber, S., Salzer, J. L., Trimmer, J. S., Shrager, P., Peles, E. (1999). Caspr2, a new member of the neurexin superfamily, is localized at the juxtaparanodes of myelinated axons and associates with K+ channels. Neuron 24, 1037-47.
Poliak et al., Localization of Caspr2 in myelinated nerves depends on axon-glia interactions and the generation of barriers along the axon, J Neurosci. 21(19):7568-75 (2001).
Poliak, S., Peles, E. (2003). The local differentiation of myelinated axons at nodes of Ranvier. Nat. Rev. Neurosci. 4, 968-80.
Poliak et al., Juxtaparanodal clustering of Shaker-like K+ channels in myelinated axons depends on Caspr2 and TAG-1, J Cell Biol. 162(6):1149-60(2003).
Ramiro-Cortés, Y., Israely, I. (2013). Long lasting protein synthesis- and activity-dependent spine shrinkage and elimination after synaptic depression. PLoS One. 8, e71155.
Rubenstein, J. L., Merzenich, M. M. (2003). Model of autism: increased ratio of excitation/inhibition in key neural systems. Genes Brain. Behav. 2, 255-67.
Ruvinsky I & Meyuhas O. Ribosomal protein S6 phosphorylation: form protein synthesis to cell size. Trends Biochem Sci 31:342-8 (2006).
Sarnat, H. B., Flores-Sarnat, L. (2013). Radial microcolumnar cortical architecture: maturational arrest or cortical dysplasia? Pediatr. Neurol. 48, 259-70.
Schapiro, M. B., Murphy, D. G., Hagerman, R. J., Azari, N. P., Alexander, G. E., Miezejeski, C. M., Hinton, V. J., Horwitz, B., Haxby, J. V., Kumar, A., et al. (1995). Adult fragile X syndrome: neuropsychology, brain anatomy, and metabolism. Am. J. Med. Genet. 60, 480-93.
Shor et al., (2010). “Requirement of the mTOR kinase for the regulation of Maf1 phosphorylation and control of RNA polymerase III-dependent transcription in cancer cells,” J Biol Chem. 285(20):15380-15392.
Silverman, J. L., Yang, M., Lord, C., Crawley, J. N. (2010). Behavioural phenotyping assays for mouse models of autism. Nat. Rev. Neurosci. 11, 490-502.
Stein, M. B., Yang, B. Z., Chavira, D. A., Hitchcock, C. A., Sung, S. C., Shipon-Blum, E., Gelernter, J. (2011). A common genetic variant in the neurexin superfamily member CNTNAP2 is associated with increased risk for selective mutism and social anxiety-related traits. Biol. Psychiatry 69, 825-31.
Strauss K A, Puffenberger E G, Huentelman M J, Gottlieb S, Dobrin S E, Parod J M, Stephan D A, Morton D H. Recessive symptomatic focal epilepsy and mutant contactin-associated protein-like 2. N Engl J Med 354:1370-7 (2006).
Talos, D. M., Fishman, R. E., Park, H., Folkerth, R. D., Follett, P. L., Volpe, J. J., Jensen, F. E. (2006). Developmental regulation of alpha-amino-3-hydroxy-5-methyl-4-isoxazole-propionic acid receptor subunit expression in forebrain and relationship to regional susceptibility to hypoxic/ischemic injury. I. Rodent cerebral white matter and cortex. J. Comp. Neurol. 497, 42-60.
Talos, D. M., Kwiatkowski, D. J., Cordero, K., Black, P. M., Jensen, F. E. (2008). Cell-specific alterations of glutamate receptor expression in tuberous sclerosis complex cortical tubers. Ann. Neurol. 63, 454-65.
Tan G C, et al. Normal variation in fronto-occipital circuitry and cerebellar structure with an autism-associated polymorphism of CNTNAP2. Neuroimage 53:1030-42 (2010).
Thanos, P. K., Wang, G. J., Volkow, N. D. (2008). Positron emission tomography as a tool for studying alcohol abuse. Alcohol Res. Health 31, 233-7.
Thomas, G. V., Tran, C., Mellinghoff, I. K., Welsbie, D. S., Chan, E., Fueger, B., Czernin, J., Sawyers, C. L. (2006). Hypoxia-inducible factor determines sensitivity to inhibitors of mTOR in kidney cancer. Nat. Med. 12, 122-7.
Tsai, P. T., Hull C., Chu, Y-X., Green-Colozzi, E., Sadowski, A. r., Leech, J. M., Steinberg, J., Crawley, J. N., Regehr, W. C., Sahin, M. (2012) Nature 488, 647-652.
Vernes, S. C., Newbury, D. F., Abrahams, B. S., Winchester, L., Nicod, J., Groszer, M., Alarcón, M., Oliver, P. L., Davies, K. E., Geschwind, D. H., et al. (2008). A functional genetic link between distinct developmental language disorders. N. Engl. J. Med. 359, 2337-45.
Verkerk A J, et al. CNTNAP2 is disrupted in a family with Gilles de la Tourette syndrome and obsessive compulsive disorder. Genomics 82:1-9 (2003).
Wang K S, et al. A genome-wide meta-analysis identifies novel loci associated with schizophrenia and bipolar disorder. Schizophr Res 124:192-9 (2010).
Weissman, T., Noctor, S. C., Clinton, B. K., Honig, L. S., Kriegstein, A. R. (2003). Neurogenic radial glial cells in reptile, rodent and human: from mitosis to migration. Cereb Cortex. 13, 550-9.
Wilson, B. M., Cox, C. L. (2007). Absence of metabotropic glutamate receptor-mediated plasticity in the neocortex of fragile X mice. Proc. Natl. Acad. Sci. USA. 104, 2454-9.
Xu, B., Ionita-Laza, I., Roos, J. L., Boone, B., Woodrick, S., Sun, Y., Levy, S., Gogos, J. A., Karayiorgou, M. (2012). De novo gene mutations highlight patterns of genetic and neural complexity in schizophrenia. Nat. Genet. 44, 1365-9.
Yizhar, O., Fenno, L. E., Prigge, M., Schneider, F., Davidson, T. J., O'Shea, D. J., Sohal, V. S., Goshen, I., Finkelstein, J., Paz, J. T., et al. (2011). Neocortical excitation/inhibition balance in information processing and social dysfunction. Nature 477, 171-8.
Yu, K., Shi, C., Toral-Barza, L., Lucas, J., Shor, B., Kim, J. E., Zhang, W. G., Mahoney, R., Gaydos, C., Tardio, L., et al. (2010). Beyond rapalog therapy: preclinical pharmacology and antitumor activity of WYE-125132, an ATP-competitive and specific inhibitor of mTORC1 and mTORC2. Cancer Res. 70, 621-31.
Yu, T. W., Chahrour, M. H., Coulter, M. E., Jiralerspong, S., Okamura-Ikeda, K., Ataman, B., Schmitz-Abe, K., Harmin, D. A., Adli, M., Malik, A. N., et al. (2013). Using whole-exome sequencing to identify inherited causes of autism. Neuron 77, 259-73.
Yu and Toral-Barza, “Biochemical and pharmacological inhibition of mTOR by rapamycin and an ATP-competitive mTOR inhibitor”, Chapter 2 in Weichart, ed. “mTOR: Methods and Protocols, Methods in Molecular Biology vol 821, pp. 15-26 (2012).
Zhou J, et al. Pharmacological inhibition of mTORC1 suppresses anatomical, cellular, and behavioral abnormalities in neural-specific Pten knock-out mice. J Neurosci 29:1773-83 (2009).

Various publications are cited herein, the contents of which are hereby incorporated by reference in their entireties.

What is claimed is:

1. A method of treating a neurodevelopmental or neuropsychiatric disorder in a subject comprising administering, to a subject in need of such treatment, a therapeutically effective amount of an mTOR kinase inhibitor.

2. The method of claim 1, where the neurodevelopmental or neuropsychiatric disorder is schizophrenia (SCZ), autism spectrum disorder (ASD), bipolar disorder, attention-deficit hyperactivity disorder (ADHD), Gilles de la Tourette disorder, obsessive-compulsive disorder, depression, mood disorders, seizure disorder, cognitive dysfunction or mental retardation.

3. The method of claim 1, where the mTOR kinase inhibitor is ATP-competitive.

4. The method of claim 3, where the mTOR kinase inhibitor is a pyrazolopyrimidine ATP-competitor.

5. The method of claim 4, where the mTOR kinase inhibitor is a pyrazolopyrimidine substituted with a bridged morpholine ATP-competitor.

6. The method of claim 5, where the mTOR kinase inhibitor is WYE-125132.

7. The method of claim 3, where the mTOR kinase inhibitor has the general formula I: 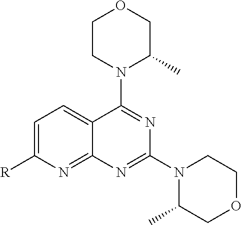
where R is a substituted or unsubstituted aromatic, for example a substituted or unsubstituted phenyl, where when present the one or more substituent may be, independently, a halogen such as fluorine, chlorine or bromine, a hydroxyl, a C1-C4 alkoxy, or a substituted or unsubstituted amide.

8. The method of claim 7, where, in the mTOR kinase inhibitor, R is

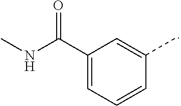

9. The method of claim 7, where, in the mTOR kinase inhibitor, R is

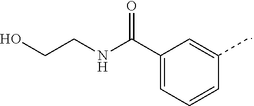

10. The method of claim 3, where the mTOR kinase inhibitor has the general formula II: 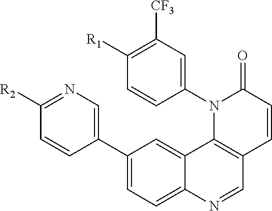
where R₁ may be H, or C₁-C₄ alkyl, or substituted or unsubstituted amino, or a 3-6 member aliphatic or aromatic ring, which may optionally be a heterocycle comprising at least one N where said ring may be substituted or unsubstituted, where when present the one or more substituent may be, independently, a halogen such as fluorine, chlorine or bromine, a hydroxyl, a C₁-C₄ alkoxy, or a substituted or unsubstituted amide; and where R₂ is a substituted or unsubstituted amine, a halogen such as fluorine, chlorine or bromine, a hydroxyl, or a C₁-C₄ alkoxy, where a substituent may be, for example, C₁-C₄ alkyl.

11. The method of claim 10, where the mTOR kinase inhibitor is Torin 2 having the structure:

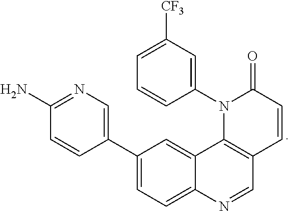 [SMILES: Nc1ccc(-c2ccc3ncc4ccc(=O)n(-c5cccc(C(F)(F)F)c5)c4c3c2)cn1]

12. A pharmaceutical composition comprising a mTOR kinase inhibitor, for use in treating a neurodevelopmental or neuropsychiatric disorder.

13. The composition of claim 12, where the neurodevelopmental or neuropsychiatric disorder is schizophrenia (SCZ), autism spectrum disorder (ASD), bipolar disorder, attention-deficit hyperactivity disorder (ADHD), Gilles de la Tourette disorder, obsessive-compulsive disorder, depression, mood disorders, seizure disorder, cognitive dysfunction or mental retardation.

14. The composition of claim 13, where the mTOR kinase inhibitor is ATP-competitive.

15. The composition of claim 14, where the mTOR kinase inhibitor is a pyrazolopyrimidine ATP-competitor.

16. The composition of claim 15, where the mTOR kinase inhibitor is a pyrazolopyrimidine substituted with a bridged morpholine ATP-competitor.

17. The composition of claim 16, where the mTOR kinase inhibitor is WYE-125132.

18. The composition of claim 14, where the mTOR kinase inhibitor has the general formula 1: 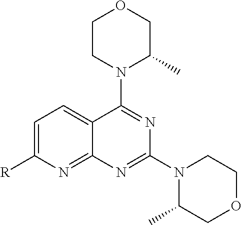
where R is a substituted or unsubstituted aromatic, for example a substituted or unsubstituted phenyl, where when present the one or more substituent may be, independently, a halogen such as fluorine, chlorine or bromine, a hydroxyl, a C1-C4 alkoxy, or a substituted or unsubstituted amide.

19. The composition of claim 18, where, in the mTOR kinase inhibitor, R is

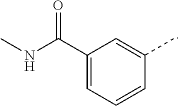

20. The composition of claim 18, where, in the mTOR kinase inhibitor, R is

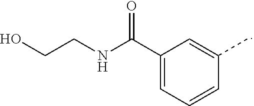

21. The composition of claim 14, where the mTOR kinase inhibitor has the general formula II: 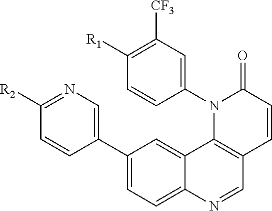
where R₁ may be H, or C₁-C₄ alkyl, or substituted or unsubstituted amino, or a 3-6 member aliphatic or aromatic ring, which may optionally be a heterocycle comprising at least one N where said ring may be substituted or unsubstituted, where when present the one or more substituent may be, independently, a halogen such as fluorine, chlorine or bromine, a hydroxyl, a C₁-C₄ alkoxy, or a substituted or unsubstituted amide; and where R₂ is a substituted or unsubstituted amine, a halogen such as fluorine, chlorine or bromine, a hydroxyl, or a C₁-C₄ alkoxy, where a substituent may be, for example, C₁-C₄ alkyl.

22. The composition of claim 21, where the mTOR kinase inhibitor is Torin 2 having the structure:

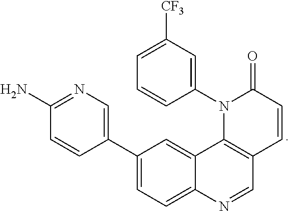 [SMILES: Nc1ccc(-c2ccc3ncc4ccc(=O)n(-c5cccc(C(F)(F)F)c5)c4c3c2)cn1]

23. A method of treating a subject having a neurodevelopmental or neuropsychiatric disorder, comprising:
a) determining whether the subject manifests a hyperactivity of the mTOR pathway; and
b) where hyperactivity of the mTOR pathway is present, treating the subject with a mTOR kinase inhibitor.
